(19) United States
(12) Patent Application Publication
(10) Pub. No.: US 2024/0247024 A1
(43) Pub. Date: Jul. 25, 2024

(54) INHIBITORS OF NICOTINAMIDE N-METHYL TRANSFERASE (NNMT)

(71) Applicant: UNIVERSITEIT LEIDEN, Leiden (NL)

(72) Inventors: Nathaniel I. Martin, Leiden (NL); Yonghzi Gao, Leiden (NL); Matthijs Van Haren, Leiden (NL); Ned Buijs, Leiden (NL); Richard Bramwell Parsons, London (GB); Monica Emanuelli, Ancona (IT); Davide Sartini, Ancona (IT)

(73) Assignee: UNIVERSITEIT LEIDEN, Leiden (NL)

(21) Appl. No.: 18/553,278

(22) PCT Filed: Mar. 30, 2022
(86) PCT No.: PCT/NL2022/050176

§ 371 Date: Sep. 29, 2023

(30) Foreign Application Priority Data
Mar. 30, 2021 (NL) 2027866

(51) Int. Cl. C07H 19/173; A61K 31/7076; A61P 35/00

(52) U.S. Cl. CPC C07H 19/173; A61K 31/7076; A61P 35/00

(57) ABSTRACT

This invention relates to compounds that are useful as inhibitors, in particular as inhibitors of Nicotinamide N-methyltransferase (NNMT), and formulations composing such compounds. The compounds and formulations may be used as a medicament, for example in the treatment of cancer, metabolic disease, or neurodegenerative disease. The compounds may be of formula (I).

This invention relates to inhibitors of Nicotinamide N-methyl Transferase (NNMT). Also provided are methods of producing these compounds and uses of these compounds.

### BACKGROUND

Nicotinamide N-methyltransferase (NNMT) methylates nicotinamide to generate 1-methyl nicotinamide. Since its discovery 70 years ago, the appreciation of NNMT's role in human health has evolved from serving only metabolic functions to also being a driving force in disease, including a variety of cancers. Despite the increasing amount of evidence indicating NNMT as a viable therapeutic target, the development of cell-active inhibitors against this enzyme is lacking.

Nicotinamide N-methyltransferase (NNMT) (EC 2.1.1.1) is a phase II metabolizing enzyme that belongs to the family of S-adenosyl-1-methionine (SAM)-dependent methyltransferases (Martin J L, McMillan F M. SAM (dependent) I AM: the S-adenosylmethionine-dependent methyltransferase fold. Curr Opin Struct Biol 2002; 12: 783-93). In 1951, NNMT was first partially purified from rat liver by Cantoni, who subsequently discovered the structure of cofactor SAM in 1952.

NNMT catalyzes the methylation of nicotinamide (NA) and a variety of other pyridine containing compounds using the methyl donor SAM to generate S-adenosyl-1-homocysteine (SAH) and 1-methyl nicotinamide (MNA) or the corresponding pyridinium ion, according to the following reaction (Alston T A, Abeles R H. Substrate specificity of nicotinamide methyltransferase isolated from porcine liver. Arch Biochem Biophys 1988; 260: 601-8; van Haren M J, Sastre Torano J, Sartini D, Emanuelli M, Parsons R B, Martin N I. A Rapid and Efficient Assay for the Characterization of Substrates and Inhibitors of Nicotinamide N-Methyltransferase. Biochemistry 2016; 55: 5307-15):

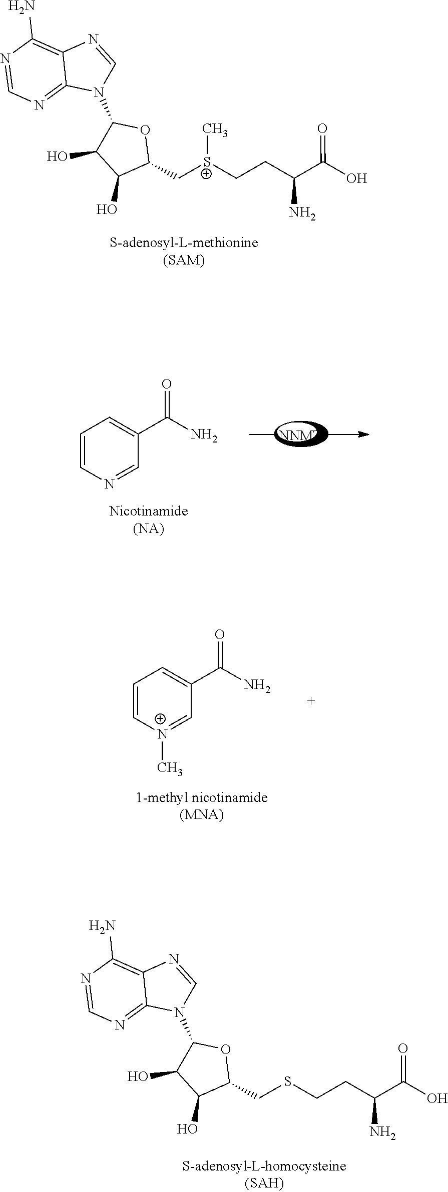

NNMT is found predominantly in the liver, but low levels of NNMT are also detected in most other organs (Aksoy S, Szumlanski C L, Weinshilboum R M. Human liver nicotinamide N-methyltransferase. cDNA cloning, expression, and biochemical characterization. J Biol Chem 1994; 269: 14835-40). It was originally thought that the primary roles of NNMT were centered around NA metabolism and detoxification of xenobiotic compounds (Pissios P. Nicotinamide N-Methyltransferase: More Than a Vitamin B3 Clearance Enzyme. Trends Endocrinol Metab 2017; 28: 340-53). However, more recent studies have provided evidence pointing towards a much broader function for NNMT in both healthy and disease states. NNMT is involved in the regulation of the cellular level of SAM as well as the SAM/SAH ratio. Not only does NNMT consume SAM, but it also promotes SAM regeneration from homocysteine through interactions with betaine-homocysteine methyltransferase and methionine adenosyltransferase, both of which play key roles in the methionine cycle (Hong S, Zhai B, Pissios P. Nicotinamide N-Methyltransferase Interacts with Enzymes of the Methionine Cycle and Regulates Methyl Donor Metabolism. Biochemistry 2018; 57: 5775-9). Furthermore, NNMT plays a critical part in NAD-dependent signaling and links the NAD+ and methionine metabolism pathways through parallel depletion of NA and SAM (Komatsu M, et al. NNMT activation can contribute to the development of fatty liver disease by modulating the NAD+ metabolism. Sci Rep 2018; 8: 1-15; and Bockwoldt M, et al., Identification of evolutionary and kinetic drivers of NAD-dependent signaling. Proc Natl Acad Sci USA 2019; 116: 15957-66). Through these pathways, NNMT modulates energy expenditure in adipose tissue and controls glucose, cholesterol and triglyceride metabolism in hepatocytes through interaction with sirtuins (Hong S, et al. Nicotinamide N-methyltransferase regulates hepatic nutrient metabolism through Sirt1 protein stabilization. Nat Med 2015; 21: 887-94). Notably, in a C. elegans model, the activity of NNMT was found to extend lifespan by decreasing cellular SAM levels, producing a starvation signal and consequently inducing autophagy. Simultaneously, the MNA thereby formed is oxidized leading to the release of reactive oxygen species, thereby increasing stress resistance and promoting longevity (Schmeisser K, et al. Role of sirtuins in lifespan regulation is linked to methylation of nicotinamide. Nat Chem Biol 2013; 9: 693-700; and Schmeisser K, Parker J A. Nicotinamide-N-methyltransferase controls behavior, neurodegeneration and lifespan by regulating neuronal autophagy. PLOS Genet 2018; 14: e1007561).

The elucidation of the various functions of NNMT demonstrates the complexity of the pathways in which the enzyme is involved. Not surprisingly, aberrant NNMT expression leads to a wide range of disorders and diseases. Most pronounced in this regard is the overexpression of NNMT in a number of human cancers (see, for example, Pissios P. Nicotinamide N-Methyltransferase: More Than a Vitamin B3 Clearance Enzyme. Trends Endocrinol Metab 2017; 28: 340-53; Ramsden D B, et al. Nicotinamide N-Methyltransferase in Health and Cancer. Int J Tryptophan Res 2017; 10: 117864691769173; and Lu X M, Long H. Nicotinamide N-methyltransferase as a potential marker for cancer. Neoplasma 2018; 65: 656-63).

A second disease area with increased interest in NNMT as a therapeutic target are metabolic disorders. Aside from the clearly emerging roles in cancer and metabolic disease, links to aberrant NNMT expression have also been found in neurodegenerative diseases, as well as functional disorders of the endothelium.

Examples of known NNMT inhibitors may be SAM Competitive Inhibitors. The byproduct SAH (1, FIG. 1), common to all SAM-dependent methyltransferases, is known as a feedback inhibitor and inhibits NNMT with an IC50 value of 35.3 μM (van Haren M J, et al. Inhibitors of nicotinamide N-methyltransferase designed to mimic the methylation reaction transition state. Org Biomol Chem 2017; 15: 6656-67). SAH is only active in enzyme-based biochemical assays; it loses its activity in cellular assays where it is rapidly degraded by S-adenosyl-L-homocysteine hydrolase (SAHH) to adenosine and homocysteine. Another known general methyltransferase inhibitor is the natural product sinefungin (2, FIG. 1), a SAM-mimicking methyltransferase inhibitor isolated from Streptomyces. Sinefungin is a moderate inhibitor of NNMT with an IC50 of 12.5 μM. Sinefungin has low cell membrane permeability and exhibits severe toxicity in animal models, restricting its potential application as a therapeutic agent (Zweygarth E, et al. Evaluation of sinefungin for the treatment of Trypanosoma (Nannomonas) congolense infections in goats. Trop Med Parasitol 1986; 37: 255-7). The moderate inhibitory activity of the SAM-mimics like SAH and sinefungin suggests that interactions in the SAM binding site alone are not sufficient for potent and selective inhibition of NNMT.

Nicotinamide Competitive Inhibitors, i.e. inhibitors that compete with binding of the nicotinamide substrate have also been reported. As described above for the NNMT byproduct SAH, the other enzymatic product, namely the methylated pyridine product MNA (3, FIG. 1) is also a feedback inhibitor of NA methylation with comparable potency to that of SAH (IC50=24.6 μM). Similar levels of inhibition are observed for other N-methylated products formed from other substrate heterocycles including the N-methylated quinoline, 1-MQ (4, FIG. 1) which exhibits an IC50 value of 12.1 μM. In a structure-activity relationship (SAR) study involving various methylated quinolines, both 5-amino-1-MQ (5, IC50=1.2 μM) and 8-methyl-1-MQ (6, IC50=1.8 μM were shown to have improved inhibition compared with the parent compound (Neelakantan H, et al. Structure-Activity Relationship for Small Molecule Inhibitors of Nicotinamide N-Methyltransferase. J Med Chem 2017; 60: 5015-28). Furthermore, in an aged mouse model, compound 5 was found to accelerate muscle regeneration, linking NNMT inhibition to functional improvements of aged skeletal muscles (Neelakantan H, et al. Small molecule nicotinamide N-methyltransferase inhibitor activates senescent muscle stem cells and improves regenerative capacity of aged skeletal muscle. Biochem Pharmacol 2019; 163: 481-92). In addition, treatment of diet-induced obese (DIO) mice with compound 5 resulted in significantly reduced body weight and white adipose mass, decreased adipocyte size, and lowered plasma total cholesterol levels (Neelakantan H, et al. Selective and membrane-permeable small molecule inhibitors of nicotinamide N-methyltransferase reverse high fat diet-induced obesity in mice. Biochem Pharmacol 2018; 147: 141-52).

A study by Ruf and colleagues (Ruf S, et al. Novel nicotinamide analog as inhibitor of nicotinamide N-methyltransferase. Bioorganic Med Chem Lett 2018; 28: 922-5) identified JBSNF-000088 (7, FIG. 1) which showed low micromolar activity against NNMT (IC50=2.4 μM), which was improved after a SAR study to JBSNF-000265 (8, FIG. 1, IC50=0.59 μM). Crystal structures show that compound 7 is methylated by NNMT in the nicotinamide binding site, which indicates the compounds are acting as slow turnover substrates. In high-fat DIO mice, compound 7 was able to reduce plasma levels of MNA, improve insulin sensitivity, normalize glucose tolerance, and reduce body weight.[28]

Based on the inhibitory activities of compounds that exclusively target either the SAM or NA binding pocket, it becomes apparent that targeting only one of these pockets may not be sufficient to achieve potent inhibition of NNMT. As an alternative, bisubstrate NNMT inhibitors have been designed to simultaneously engage both of these binding pockets. From the SAR performed, it became clear that many of the functional groups present in SAM and NA are essential for binding and small alterations in the chemical structure of the bisubstrate compounds can have significant impact on their activity. The bisubstrate inhibitor MvH45 (9, FIG. 1) linked a benzamide, mimicking NA, to an Aza-SAH moiety, mimicking SAM, resulting in moderate inhibition of NNMT (IC50=29.2 μM) (van Haren M J, et al. Inhibitors of nicotinamide N-methyltransferase designed to mimic the methylation reaction transition state. Org Biomol Chem 2017; 15: 6656-67). Building on this result, Jin et al. extended the linker to the benzamide from one to two carbon atoms resulting in MS2756 (compound 10, FIG. 1) which exhibited a significantly reduced activity (IC50=160 μM). Interestingly, extension of the linker to the amino acid moiety by one carbon as in MS2734 (compound 11, FIG. 1), led to a restoration of inhibitory activity (IC50=14 μM) (Babault N, et al. Discovery of Bisubstrate Inhibitors of Nicotinamide N-Methyltransferase (NNMT). J Med Chem 2018; 61: 1541-51).

Optimization of the structural features of these bisubstrate inhibitors led us to pursue an SAR focusing on the amino acid and benzamide side-chains. From this work a naphthalene-containing compound (GYZ191 (12), FIG. 1) emerged with an IC50 of 1.4 μM. Cellular data obtained for compound 12 showed a significant inhibitory effect on cell viability in HSC-2 oral cancer cells (Gao Y, et al. Bisubstrate inhibitors of nicotinamide N-methyltransferase (NNMT) with enhanced activity. J Med Chem 2019; 62: 6597-614). The group of Shair identified other NNMT inhibitors (Ki=0.5 nM for compound NS1 13, FIG. 1) (Policarpo R L, et al. High-Affinity Alkynyl Bisubstrate Inhibitors of Nicotinamide N-Methyltransferase (NNMT). J Med Chem 2019; 62: 9837-73). However, in cell-based assays, both 13 and its methyl ester prodrug only moderately decreased MNA levels in U2OS cells, most likely due to limited cell permeability. Following a similar strategy, Huang and co-workers synthesized LL320 (compound 14, FIG. 1) which provided a Ki value of 1.6 nM (Chen D, et al. Novel Propargyl-Linked Bisubstrate Analogues as Tight-Binding Inhibitors for Nicotinamide N-Methyltransferase. J Med Chem 2019; 62: 10783-97). Good selectivity was also observed against a panel of small molecule, lysine and arginine methyltransferases. As for the other SAM-based bisubstrate inhibitors of NNMT, however, both LL320 and its ethyl ester prodrug displayed poor cell permeability.

A number of covalent inhibitors have also been identified. The active site of NNMT contains several non-essential cysteine residues, which can be explored as targets for covalent inhibition. A number of exemplary covalent inhibitors are illustrated in FIG. 1, including RS004 (15, FIG. 1; in Horning B D, et al. Chemical Proteomic Profiling of Human Methyltransferases. J Am Chem Soc 2016; 138: 13335-43), HS58a-C2 and HS312 (16, and 17, FIG. 1; in Lee H-Y, et al. Covalent inhibitors of nicotinamide N-methyltransferase (NNMT) provide evidence for target engagement challenges in situ. Bioorg Med Chem Lett 2018; 28: 2682-7); and several 4-chloropyridine analogues (compounds 18-20, FIG. 1; in Sen S, et al. Development of a Suicide Inhibition-Based Protein Labeling Strategy for Nicotinamide N-Methyltransferase. ACS Chem Biol 2019; 14: 613-8).

While the search for effective NNMT inhibitors is still in its infancy, progress has already been made in terms of potency and selectivity of small molecule inhibitors of NNMT. That said, the limited cellular and in vivo activity of these compounds speaks to the need to develop further inhibitors. The clinical importance of NNMT in a variety of diseases, including cancer and metabolic disorders, supports NNMT as a viable therapeutic target. However, major challenges remain in developing NNMT inhibitors for clinical application.

An object of the invention is to provide compounds that are useful as inhibitors of NNMT.

### SUMMARY OF THE INVENTION

The invention provides compounds that are useful as inhibitors of NNMT. In accordance with the present invention there is provided in a first aspect:

A compound of formula I,

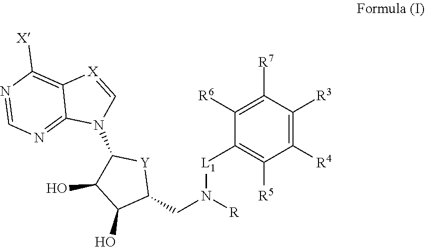

wherein:
X is selected from N and CH;
X′ is selected from NH₂ and halo;
Y is selected from O and CH₂;
L₁ is selected from the group consisting of: C₂-C₅ alkyl, C₃-C₅ alkenyl and C₃-C₅ alkynyl;
R is selected from the group consisting of:

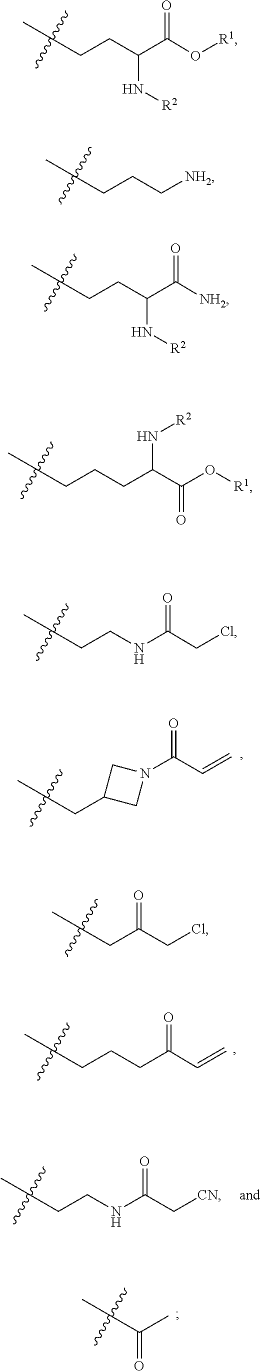

R¹ is selected from the group consisting of: H and a masking group;
R² is selected from the group consisting of: H and a masking group;
R³ is selected from the group consisting of: H and an electron withdrawing group;
when R³ is an electron withdrawing group, R⁴ is selected from H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g}; and when R³ is H, R⁴ is selected from C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g};
R⁵, R⁶ and R⁷ are each independently selected from H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g};
R^{a}, R^{b}, R^{c}, R^{d}, R^{e}, R^{f} and R^{g} are each independently at each occurrence selected from the group consisting of: H, halo, C₁-C₃ alkyl, C₂-C₅ alkenyl, and C₁-C₃ haloalkyl;
or a pharmaceutically acceptable salt, stereoisomer, solvate or prodrug thereof.

In an embodiment the compound is a compound of formula IA, or a pharmaceutically acceptable salt, stereoisomer, solvate or prodrug thereof:

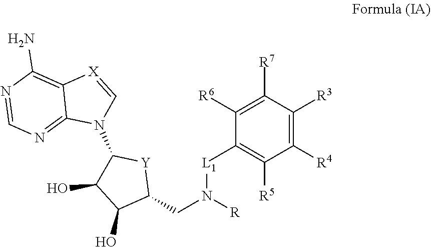

wherein R is selected from the group consisting of:

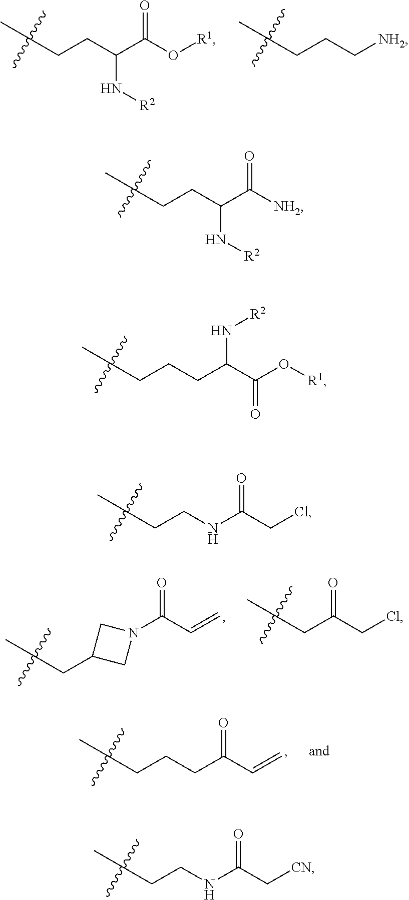

and
X, Y, L₁, R¹, R², R³, R⁴, R⁵, R⁶, and R⁷ are as defined for Formula (I).

In accordance with the present invention, there is provided in a second aspect a formulation comprising a compound of the invention and optionally a pharmaceutically acceptable carrier.

A third aspect provides a compound or formulation of the invention for use as a medicament.

A fourth aspect provides a compound or formulation of the invention for use in the treatment of cancer.

A fifth aspect provides a compound or formulation of the invention for use in the treatment of metabolic disease.

A sixth aspect provides a compound or formulation of the invention for use in the treatment of neurodegenerative disease.

A seventh aspect provides a compound or formulation of the invention for use in the treatment of a functional disorder of the endothelium.

An eighth aspect provides a compound or formulation of the invention for use in the treatment of a condition treated by the inhibition of Nicotinamide N-methyltransferase (NNMT).

A ninth aspect provides a method of treatment of a condition which is modulated by the inhibition of NNMT, wherein the method comprises administering a therapeutic amount of a compound or formulation of the invention to a patient in need thereof.

A tenth aspect provides the use of a compound or formulation of the invention for the inhibition of NNMT in vitro or in vivo.

The invention will now be described further by reference to the following examples and figures. These are not intended to be limiting but merely exemplary of the invention.

### BRIEF DESCRIPTION OF THE DRAWINGS

Embodiments of the invention are further described hereinafter with reference to the accompanying drawings, in which:

FIG. 1 illustrates the structures of exemplary known NNMT inhibitors, including methyltransferase specific inhibitors 1 and 2, nicotinamide competitive inhibitors 3-8, bisubstrate inhibitors 9-14 and covalent inhibitors 15-20.

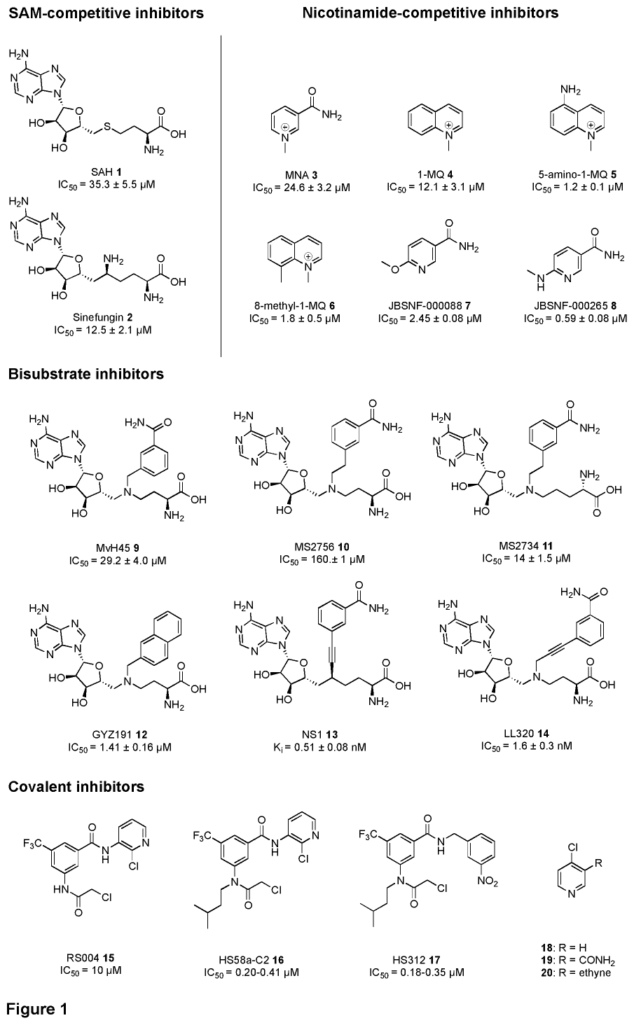

FIG. 2 provides exemplary structures of proteolysis targeting chimeras (PROTACs), where a high affinity NNMT inhibitor is attached via linkers of varying lengths to a “Flag” that is either an E3 ligase ligands (1 and 2) or hydrophobic tags (3 and 4).

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

All publications, patent applications, patents, and other references mentioned herein are incorporated by reference in their entirety. In case of conflict, the present specification, including definitions, will control.

### Definitions

The following explanations of terms and methods are provided to better describe the present disclosure and to guide those of ordinary skill in the art in the practice of the present disclosure.

The invention concerns amongst other things the treatment of a disease. The term “treatment”, and the therapies encompassed by this invention, include the following and combinations thereof: (1) hindering, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission). The benefit to a patient to be treated may be either statistically significant or at least perceptible to the patient or to the physician. It will be understood that a medicament will not necessarily produce a clinical effect in each patient to whom it is administered; thus, in any individual patient or even in a particular patient population, a treatment may fail or be successful only in part, and the meanings of the terms “treatment”, “prophylaxis” and “inhibitor” and of cognate terms are to be understood accordingly. The compositions and methods described herein are of use for therapy and/or prophylaxis of the mentioned conditions.

The term “prophylaxis” includes reference to treatment therapies for the purpose of preserving health or inhibiting or delaying the initiation and/or progression of an event, state, disorder or condition, for example for the purpose of reducing the chance of an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

The term “inhibit” (and “inhibiting”) includes reference to delaying, stopping, reducing the incidence of, reducing the risk of and/or reducing the severity of an event, state, disorder or condition. Inhibiting an event, state, disorder or condition may therefore include delaying or stopping initiation and/or progression of such, and reducing the risk of such occurring. The products of the disclosure may be used to inhibit NNMT and thereby aid in clearing bacterial infection and/or other events, disorders and/or conditions which are disclosed herein. For example, the compounds of the invention may release an NNMT inhibitor.

An “inhibitor” is a molecule that binds to an enzyme and decreases its activity. An “irreversible inhibitor” is an inhibitor where the binding involves a chemical reaction, e.g. formation of a covalent bond between the molecule and enzyme. The compounds of the invention may release an NNMT inhibitor.

The terms “alkyl” as used herein include reference to a straight or branched chain alkyl moiety having 1, 2, 3, 4, 5, 6, 7, 8, 9 or 10 carbon atoms. The term includes reference to, for example, methyl, ethyl, propyl (n-propyl or isopropyl), butyl (n-butyl, sec-butyl or tert-butyl), pentyl, hexyl and the like. In particular, alkyl may be a “C₁-C₄ alkyl”, i.e. an alkyl having 1, 2, 3 or 4 carbon atoms; or a “C₁-C₆ alkyl”, i.e. an alkyl having 1, 2, 3, 4, 5 or 6 carbon atoms; or a “C₁-C₃ alkyl”, i.e. an alkyl having 1, 2 or 3 carbon atoms. The term “lower alkyl” includes reference to alkyl groups having 1, 2, 3 or 4 carbon atoms.

The term “alkylene” by itself or as part of another substituent means a divalent radical derived from an alkyl, as exemplified, but not limited, by —CH2CH2CH2CH2-. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “cycloalkyl” as used herein includes reference to an alicyclic moiety having 3, 4, 5 or 6 carbon atoms. The group may be a bridged or polycyclic ring system. More often cycloalkyl groups are monocyclic. This term includes reference to groups such as cyclopropyl, cyclobutyl, cyclopentyl, cyclohexyl and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. For example, a heterocycloalkyl may comprise 3, 4, or 5 ring carbon atoms and 1 or 2 ring heteroatoms selected from nitrogen and oxygen. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

The terms “halo” or “halogen” as used herein includes reference to F, Cl, Br or I, for example F, Cl or Br. In a particular class of embodiments, halogen is F or Cl, of which F is more common.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “haloalkyl” refers to an alkyl group where one or more hydrogen atoms are substituted by a corresponding number of halogens. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms, e.g. 1, 2 or 3 carbon atoms. This term includes reference to, for example, methoxy, ethoxy, propoxy, isopropoxy, butoxy, tert-butoxy, pentoxy, hexoxy and the like. The term “lower alkoxy” includes reference to alkoxy groups having 1, 2, 3 or 4 carbon atoms.

The term “haloalkoxy” as used herein refers to an alkoxy group where one or more hydrogen atoms are substituted by a corresponding number of halogens.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Arylene” and “heteroarylene” refers to a divalent radical derived from a aryl and heteroaryl, respectively.

Each of the above terms (e.g., “alkyl,” “cycloalkyl,” “heteroalkyl,” “aryl” and “heteroaryl”), unless otherwise noted, are meant to include both substituted and unsubstituted forms of the indicated radical. Where a substituent is R-substituted (e.g. an Rx-substituted alkyl, where “x” is an integer), the substituent may be substituted with one or more R groups as allowed by chemical valency rules where each R group is optionally different (e.g. an Rx-substituted alkyl may include multiple Rx groups wherein each Rx group is optionally different). Certain examples of substituents for each type of radical are provided below.

The term “substituted” as used herein in reference to a moiety means that one or more, especially up to 5, more especially 1, 2 or 3, of the hydrogen atoms in said moiety are replaced independently of each other by the corresponding number of the described substituents. Unless otherwise specified, exemplary substituents include —OH, —CN, —NH₂, ═O, -halo, —C₁-C₆ alkyl, —C₂-C₆ alkenyl, —C₁-C₆ haloalkyl, —C₁-C₆ haloalkoxy and —C₂-C₆ haloalkenyl, —C₁-C₆ alkylcarboxylic acid (e.g. —CH₃COOH or —COOH). Where the substituent is a —C₁-C₆ alkyl or —C₁-C₆ haloalkyl, the C₁-C₆ chain is optionally interrupted by an ether linkage (—O—) or an ester linkage (—C(O)O—). Exemplary substituents for a substituted alkyl may include —OH, —CN, —NH₂, ═O, -halo, —CO₂H, —C₁-C₆ haloalkyl, —C₁-C₆ haloalkoxy and —C₂-C₆haloalkenyl, —C₁-C₆ alkylcarboxylic acid (e.g. —CH₃COOH or —COOH). For example, exemplary substituents for an alkyl may include —OH, —CN, —NH₂, ═O, -halo.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Where steric issues determine placement of substituents on a group, the isomer having the lowest conformational energy may be preferred.

Where a compound, moiety, process or product is described as “optionally” having a feature, the disclosure includes such a compound, moiety, process or product having that feature and also such a compound, moiety, process or product not having that feature. Thus, when a moiety is described as “optionally substituted”, the disclosure comprises the unsubstituted moiety and the substituted moiety.

Where two or more moieties are described as being “independently” or “each independently” selected from a list of atoms or groups, this means that the moieties may be the same or different. The identity of each moiety is therefore independent of the identities of the one or more other moieties.

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centres) or double bonds; the racemates, diastereomers, tautomers, geometric isomers and individual isomers are encompassed within the scope of the present invention. The compounds of the present invention do not include those which are known in the art to be too unstable to synthesize and/or isolate.

The symbol [symbol] denotes a point of attachment of a moiety to the remainder of a compound.

The term “prodrug” as used herein represents compounds which are transformed in vivo to the parent compound or other active compound, for example, by hydrolysis in blood. An example of such a prodrug is a pharmaceutically acceptable ester of a carboxylic acid. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996); and The organic chemistry of drug design and drug action by Richard B Silverman in particular pages 497 to 546; each of which is incorporated herein by reference. Compounds of the invention may represent prodrugs (e.g. comprising a latent NNMT inhibitor, such as compounds of the invention where R¹ and/or R² are not H), which release and activate the latent NNMT inhibitor.

The term “masking group” as used herein represents a moiety that may act as a protecting group. For example, in compounds of the invention one or more masking groups (such as a trimethyl lock, an alkyl, a benzyl, or similar) may be used to mask one or more hydrophilic groups, such as amine or carboxyl groups. A masking group may be a relatively more hydrophobic moiety, which increases the cell permeability of the compound comprising the masking group compared to the compound when the masking group is removed. A compound of the invention comprising one or more masking groups may be a latent NMMT inhibitor and removal of the one or more masking groups may activate the latent NNMT inhibitor.

The term “lipid” as used herein means a fatty acid group (or fatty acid comprising group) that may be incorporated in a compound (such as a compound of the invention) as a lipid ester, diglyceride or triglyceride. Exemplary lipids comprise C₄-C₁₈ fatty acid, e.g. butyrate, undecylate, laureate, palmitate, or oleate. In an example, the lipid is a C₄-C₁₈ (e.g. C₄, C₁₀, C₁₂, C₁₆, or C₁₈) fatty acid ester.

The term “pharmaceutical formulation” as used herein includes reference to a formulation comprising at least one active compound and optionally one or more additional pharmaceutically acceptable ingredients, for example a pharmaceutically acceptable carrier. Where a pharmaceutical formulation comprises two or more active compounds, or comprises at least one active compound and one or more additional pharmaceutically acceptable ingredients, the pharmaceutical formulation is also a pharmaceutical composition. Unless the context indicates otherwise, all references to a “formulation” herein are references to a pharmaceutical formulation.

The term “product” or “product of the invention” as used herein includes reference to any product containing a compound of the present invention. In particular, the term product relates to compositions and formulations containing a compound of the present invention, such as a pharmaceutical composition, for example.

The term “therapeutically effective amount” as used herein refers to an amount of a drug, or pharmaceutical agent that, within the scope of sound pharmacological judgment, is calculated to (or will) provide a desired therapeutic response in a mammal (animal or human). The therapeutic response may for example serve to cure, delay the progression of or prevent a disease, disorder or condition.

The term “electron withdrawing group” relates to an atom or group that draws electron density from neighbouring atoms towards itself, usually by resonance or inductive effects. Exemplary electron withdrawing groups include F, Cl, Br, I, CN, NO₂, CF₃ and SO₂F.

### Compounds

In one aspect, the invention provides compounds of formula I or formula IA as previously described or a pharmaceutically acceptable salt, stereoisomer, solvate or prodrug thereof.

R may be selected from the group consisting of:

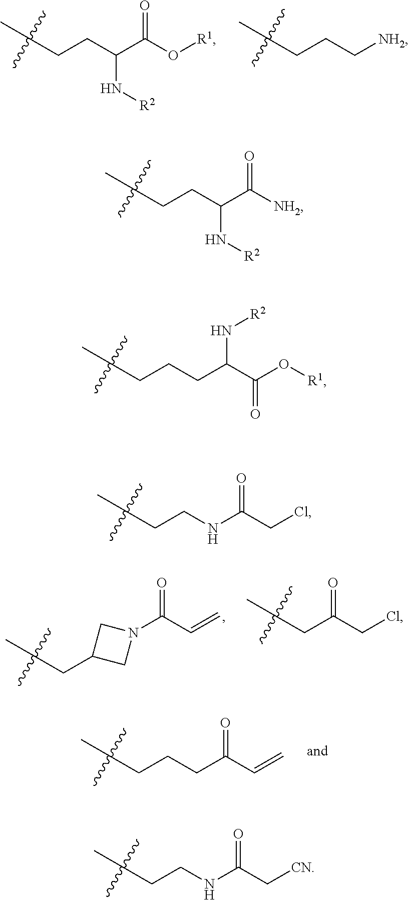

X′ may be NH₂.

X′ may be F, Cl or Br. X′ may be Cl.

L₁ may be an unsubstituted C₃-C₅ alkenyl.

L₁ may be C₁₋alkyl or C₃-alkyl. L₁ may be C₃₋alkynyl.

L₁ may be selected from the group consisting of: C₁-alkyl, C₃-alkenyl and C₃-alkynyl.

L₁ may be C₃-alkenyl. L₁ may be —CH₂CHCH—. L₁ may be —CH₂CHCH— wherein the phenyl group is situated trans to the CH₂ group of L₁.

X′ may be NH₂ and L₁ may be C₃-alkenyl. L₁ may be —CH₂CHCH—. L₁ may be —CH₂CHCH— wherein the phenyl group is situated trans to the CH₂ group of L₁.

X′ may be Cl and L₁ may be C₃-alkenyl. L₁ may be —CH₂CHCH—. L₁ may be —CH₂CHCH— wherein the phenyl group is situated trans to the CH₂ group of L₁.

R may be selected from the group consisting of:

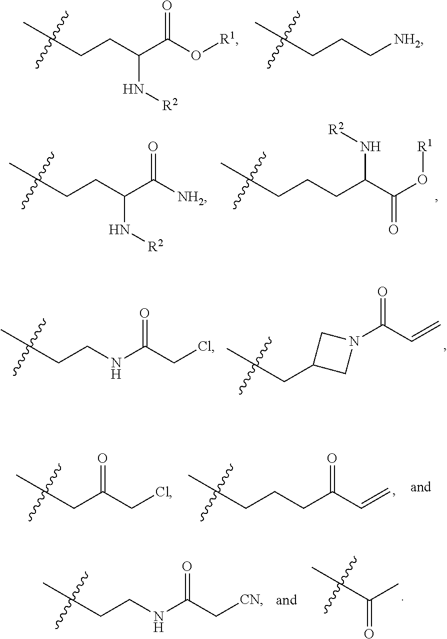

R may be selected from the group consisting of:

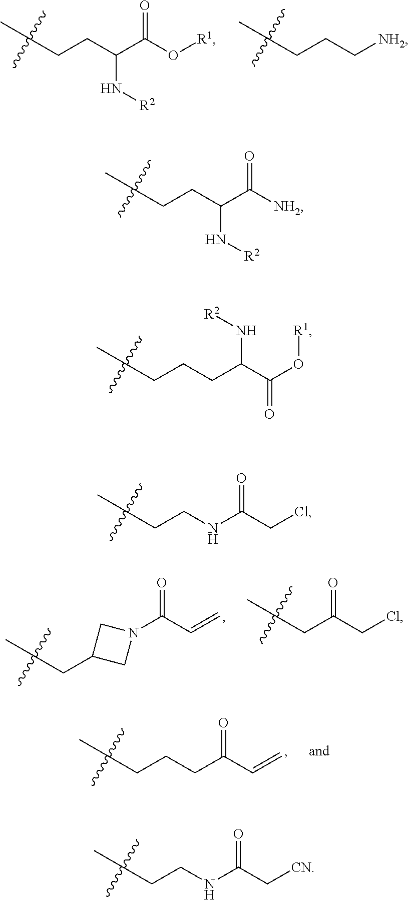

R may be selected from the group consisting of:

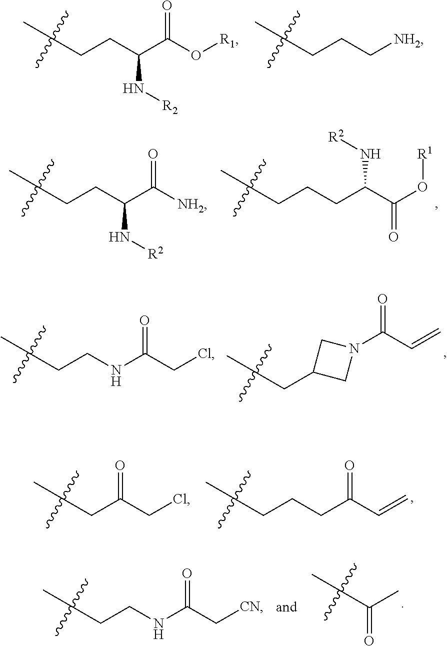

R may be selected from the group consisting of:

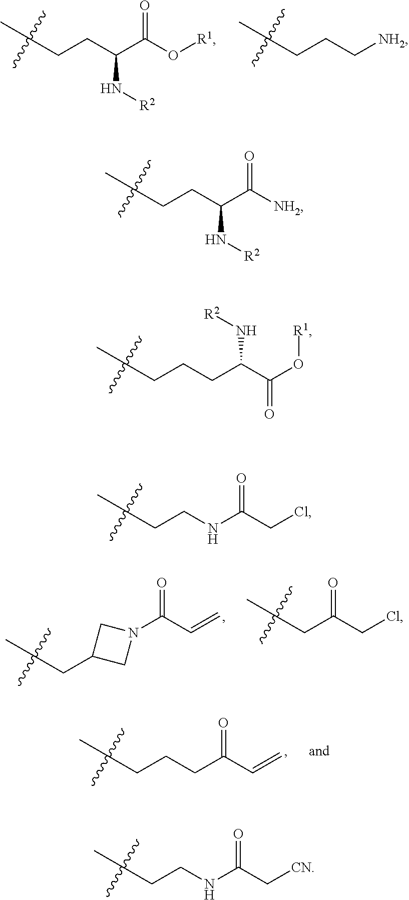

R may be

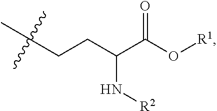

for example R may be

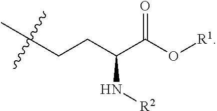

R may be

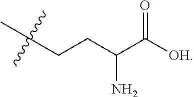 [SMILES: *CCC(N)C(=O)O]

For example, R may be

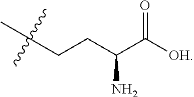 [SMILES: *CC[C@H](N)C(=O)O]

L₁ may be C₃-alkenyl and R may be

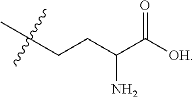 [SMILES: *CCC(N)C(=O)O]

L₁ may be —CH₂CHCH— and R may be

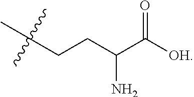 [SMILES: *CCC(N)C(=O)O]

L₁ may be —CH₂CHCH— wherein the phenyl group is situated trans to the CH₂ group of L₁ and R may be

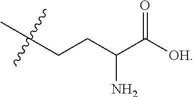 [SMILES: *CCC(N)C(=O)O]

R may be selected from

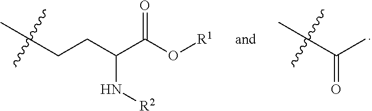

R may be selected from

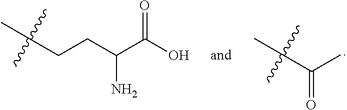

### R may be

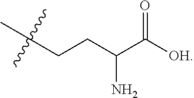 [SMILES: *CCC(N)C(=O)O]

### R may be

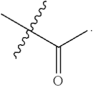 [SMILES: *C(C)=O ; "."]

R³ may be an electron withdrawing group. For exam R³ may be selected from the group consisting of: F, Cl, Br, I, CN, NO₂, CF₃ and SO₂F. R³ may be F, Cl, Br, CN, NO₂, and SO₂F. R³ may be CN.

R³ may be an electron withdrawing group. When R³ is an electron withdrawing group, R⁴ may be selected from H, halo, NO₂ and CN.

R³ may be an electron withdrawing group. When R³ is an electron withdrawing group, R⁴ may be selected from H, F, Cl, Br, I, NO₂ and CN.

R³ may be an electron withdrawing group. When R³ is an electron withdrawing group, R⁴ may be selected from H, F, NO₂ and CN.

R³ may be CN. For example, R³ may be CN, R⁴ may be F and R⁷ may be F.

R³ may be an electron withdrawing group. When R³ is an electron withdrawing group, R⁴ may be H.

X′ may be NH₂ and R³ may be an electron withdrawing group. When R³ is an electron withdrawing group, R⁴ may be H.

X′ may be Cl and R³ may be an electron withdrawing group. When R³ is an electron withdrawing group, R⁴ may be H·L₁ may be C₃-alkenyl and R³ may be an electron withdrawing group.

L₁ may be C₃-alkenyl and R³ may be CN.

X′ may be NH₂, L₁ may be C₃-alkenyl and R³ may be CN.

X′ may be Cl, L₁ may be C₃-alkenyl and R³ may be CN.

L₁ may be C₃-alkenyl and R³ may be an electron withdrawing group. When R³ is an electron withdrawing group, R⁴ may be H.

X′ may be NH₂, L₁ may be C₃-alkenyl and R³ may be an electron withdrawing group. When R³ is an electron withdrawing group, R⁴ may be H.

X′ may be Cl, L₁ may be C₃-alkenyl and R³ may be an electron withdrawing group. When R³ is an electron withdrawing group, R⁴ may be H.

L₁ may be C₃-alkenyl, R³ may be CN and R⁴ may be H.

R³ may be H. When R³ may be H, R⁴ may be selected from the group consisting of: C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R³ may be H. When R³ may be H, R⁴ may be selected from the group consisting of: C₁-C₆ alkyl, C₁-C₆ haloalkyl, halo, NO₂, CN, C(O)R^{b}, C(O)NR^{c}R^{d}, and S(O)₂R^{e}.

R³ may be H. When R³ may be H, R⁴ may be selected from the group consisting of: C₁-C₆ alkyl, C₁-C₆ haloalkyl, halo, NO₂, CN, C(O)R^{b}, C(O)NR^{c}R^{d}, and S(O)₂R^{e}.

R³ may be H. When R³ may be H, R⁴ may be selected from the group consisting of: halo, NO₂, CF₃, C(O)NR^{c}R^{d}, and CN.

R³ may be H. When R³ may be H, R⁴ may be selected from the group consisting of: F, Cl, Br, I, NO₂, CF₃ and CN.

R³ may be H. When R³ may be H, R⁴ may be selected from the group consisting of: F, NO₂, C(O)NR^{c}R^{d}, and CN.

X′ may be NH₂ and R³ may be H. When R³ is H, R⁴ may be selected from the group consisting of: F, Cl, Br, I, NO₂, CF₃ and CN.

X′ may be Cl and R³ may be H. When R³ is H, R⁴ may be selected from the group consisting of: F, Cl, Br, I, NO₂, CF₃ and CN.

When L₁ is selected from the group consisting of: C₁-C₃ alkyl and C₃ alkynyl, and R³ is H; R⁴ is not C(O)NR^{c}R^{d}. When L₁ is selected from the group consisting of: C₁-C₃ alkyl and C₃ alkynyl, and R³ is H; R⁴ is not C(O)NH₂.

X′ may be Cl, X may be N and R may be

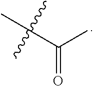 [SMILES: *C(C)=O ; "."]

### X′ may be Cl, X may be N, Y may be CH₂ and R may be

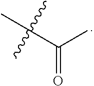 [SMILES: *C(C)=O ; "."]

### X′ may be Cl, X may be N, Y may be O and R may be

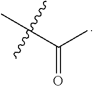 [SMILES: *C(C)=O ; "."]

X′ may be Cl, X may be N, R may be

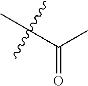 [SMILES: *C(C)=O]

and R³ may be CN. X′ may be Cl, X may be N, Y may be CH₂, R may be

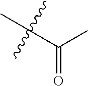 [SMILES: *C(C)=O]

### and R³ may be CN. X′ may be Cl, X may be N, Y may be O, R may be

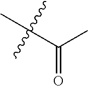 [SMILES: *C(C)=O]

### and R³ may be CN.

X′ may be NH₂, X may be N and R may be

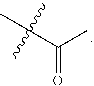 [SMILES: *C(C)=O]

X′ may be NH₂, X may be N, Y may be CH₂ and R may be

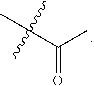 [SMILES: *C(C)=O]

### X′ may be NH₂, X may be N, Y may be O and R may be

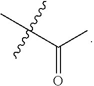 [SMILES: *C(C)=O]

X′ may be NH₂, X may be N, R may be

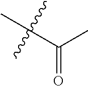 [SMILES: *C(C)=O]

and R³ may be CN. X′ may be NH₂, X may be N, Y may be CH₂, R may be

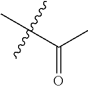 [SMILES: *C(C)=O]

and R³ may be CN. X′ may be NH₂, X may be N, Y may be O, R may be

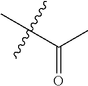 [SMILES: *C(C)=O]

### and R³ may be CN.

X′ may be Cl, X may be CH₂ may be

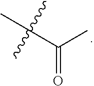 [SMILES: *C(C)=O]

X′ may be Cl, X may be CH₂, Y may be CH₂ and R may be

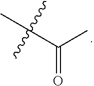 [SMILES: *C(C)=O]

### X′ may be Cl, X may be CH₂, Y may be O and R may be

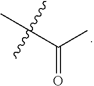 [SMILES: *C(C)=O]

X′ may be Cl, X may be CH₂, R may be

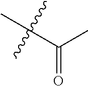 [SMILES: *C(C)=O]

and R³ may be CN. X′ may be Cl, X may be CH₂, R may be

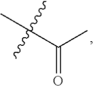 [SMILES: *C(C)=O]

Y may be CH₂ and R³ may be CN. X′ may be Cl, X may be CH₂, R may be

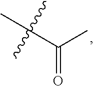 [SMILES: *C(C)=O]

### Y may be O and R³ may be CN.

X′ may be NH₂, X may be CH₂ and R may be

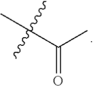 [SMILES: *C(C)=O]

X′ may be NH₂, X may be CH₂, Y may be CH₂ and R may be

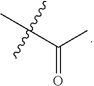 [SMILES: *C(C)=O]

X′ may be NH₂, X may be CH₂, Y may be O and R may be

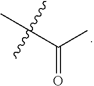 [SMILES: *C(C)=O]

X′ may be NH₂, X may be CH₂, R may be

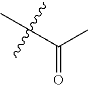 [SMILES: *C(C)=O]

and R³ may be CN. X′ may be NH₂, X may be CH₂, Y may be CH₂, R may be

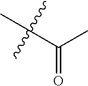 [SMILES: *C(C)=O]

and R³ may be CN. X′ may be NH₂, X may be CH₂, Y may be O, R may be

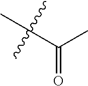 [SMILES: *C(C)=O]

### and R³ may be CN.

X′ may be NH₂, X may be N, Y may be O, L₁ may be —CH₂CHCH— and R may be

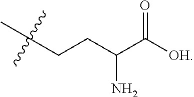 [SMILES: *CCC(N)C(=O)O]

X′ may be NH₂, X may be N, Y may be O, L₁ may be —CH₂CHCH—, R may be

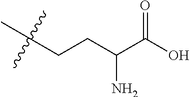 [SMILES: *CCC(N)C(=O)O]

and R³ may be CN. X′ may be NH₂, X may be N, Y may be O, L₁ may be —CH₂CHCH—, R may be

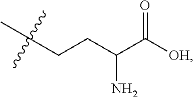 [SMILES: *CCC(N)C(=O)O]

R³ may be CN, R⁴ may be F and R⁷ may be F. X′ may be NH₂, X may be N, Y may be O, L₁ may be —CH₂CHCH—, R may be

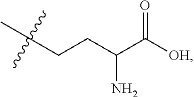 [SMILES: *CCC(N)C(=O)O]

R³ may be CN, R⁴ may be F, R⁷ may be F, R⁵ may be H and R⁶ may be H.

The compound may be a compound of formula II,

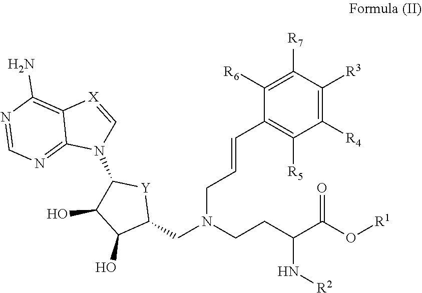

or a pharmaceutically acceptable salt, stereoisomer, solvate or prodrug thereof.

The compound may be a compound of formula IIA,

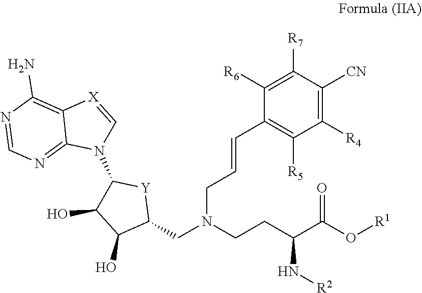

or a pharmaceutically acceptable salt, stereoisomer, solvate or prodrug thereof.

The compound may be a compound of formula IA wherein X is N and Y is O.

The compound may be a compound of formula IA wherein R¹ is H and R² is H. The compound may be a compound of formula IIA wherein R⁴ is F and R⁷ is F. The compound may be a compound of formula IA wherein R⁵ is H and R⁶ is H. The compound may be a compound of formula IA wherein R¹ is H and R² is H, R⁴ is F and R⁷ is F. The compound may be a compound of formula IIA wherein R¹ is H and R² is H, R⁴ is F, R⁷ is F, R⁵ is H and R⁶ is H.

The compound may be a compound of formula IA wherein X is N, R¹ is H and R² is H. The compound may be a compound of formula IIA wherein X is N, R⁴ is F and R⁷ is F. The compound may be a compound of formula IIA wherein X is N, R⁵ is H and R⁶ is H. The compound may be a compound of formula IIA wherein X is N, R¹ is H and R² is H, R⁴ is F and R⁷ is F. The compound may be a compound of formula IIA wherein X is N, R¹ is H and R² is H, R⁴ is F, R⁷ is F, R⁵ is H and R⁶ is H.

The compound may be a compound of formula IIA wherein X is N, Y is O, R¹ is H and R² is H. The compound may be a compound of formula IIA wherein X is N, Y is O, R⁴ is F and R⁷ is F. The compound may be a compound of formula IIA wherein X is N, Y is O, R⁵ is H and R⁵ is H. The compound may be a compound of formula IIA wherein X is N, Y is O, R¹ is H and R² is H, R⁴ is F and R⁷ is F. The compound may be a compound of formula IIA wherein X is N, Y is O, R¹ is H and R² is H, R⁴ is F, R⁷ is F, R⁵ is H and R⁶ is H·X may be N.

Y may be O.

X may be N and Y may be O.

The compound may be a compound of formula IIB,

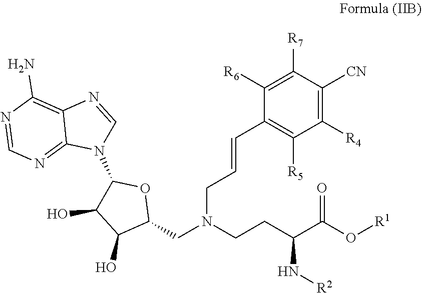

or a pharmaceutically acceptable salt, stereoisomer, solvate or prodrug thereof.

The compound may be a compound of formula IIB wherein R¹ is H and R² is H. The compound may be a compound of formula IIB wherein R⁴ is F and R⁷ is F. The compound may be a compound of formula IIB wherein R⁵ is H and R⁶ is H. The compound may be a compound of formula IIB wherein R¹ is H, R² is H, R⁴ is F and R⁷ is F. The compound may be a compound of formula IIB wherein R¹ is H, R² is H, R⁴ is F, R⁷ is F, R⁵ is H and R⁶ is H.

The compound may be a compound of formula IIC:

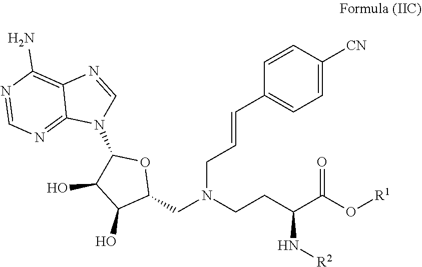

or a pharmaceutically acceptable salt, stereoisomer, solvate or prodrug thereof.

The compound may be a compound of formula IID:

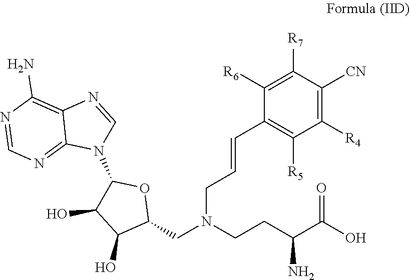

or a pharmaceutically acceptable salt, stereoisomer, solvate or prodrug thereof.

The compound may be a compound of formula IID wherein R⁴ is F and R⁷ is F. The compound may be a compound of formula IID wherein R⁵ is H and R⁶ is H. The compound may be a compound of formula IID wherein R⁴ is F, R⁷ is F, R⁵ is H and R⁶ is H.

R¹ may be selected from the group consisting of: H and a masking group, wherein the masking group is substituted or unsubstituted C₁-C₆ alkyl, lipid, substituted or unsubstituted benzyl, substituted or unsubstituted aryl, and

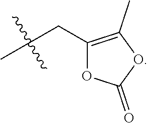 [SMILES: *Cc1oc(=O)oc1C]

R¹ may be selected from the group consisting of: H and a masking group, wherein the masking group is substituted or unsubstituted C₁-C₆ alkyl, lipids, substituted or unsubstituted benzyl, substituted or unsubstituted aryl, and

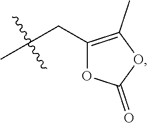 [SMILES: *Cc1oc(=O)oc1C]

wherein when R¹ is substituted, it is substituted with one to four substituents independently selected from the group consisting of oxo, halo, cyano, nitro, hydroxy, carboxy, NH₂, NH(C₁-C₂ alkyl), N(C₁-C₂ alkyl)₂, methoxy, ethoxy or aryl.

R¹ may be selected from the group consisting of: H and substituted or unsubstituted C₁-C₄ alkyl. For example, R¹ may be selected from the group consisting of methyl, ethyl, propyl, isopropyl, benzyl and H. R¹ may be H.

R¹ may be selected from the group consisting of methyl, ethyl, propyl, isopropyl, benzyl.

R² may be H.

R¹ may be H and R² may be H.

R¹ may be selected from the group consisting of methyl, ethyl, propyl, isopropyl, benzyl and R² may be H.

R² may be a masking group, optionally wherein the masking group is selected from the group consisting of:

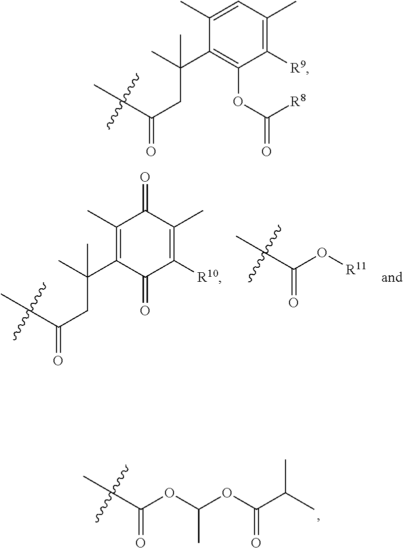

wherein
R⁸ is C₁-C₆ alkyl or aryl; R⁹ is H or methyl; R¹⁰ is H or methyl; and R¹¹ is C₁-C₆ alkyl or aryl.

R² may be a masking group. The masking group may be

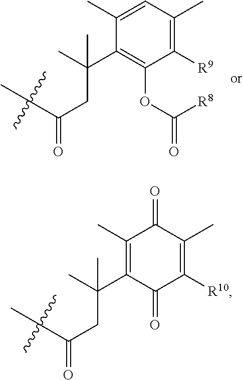

wherein R⁸ is C₁-C₆ alkyl or aryl; R⁹ is H or methyl; R¹⁰ is H or methyl.

R² may be a masking group. The masking group R² may be

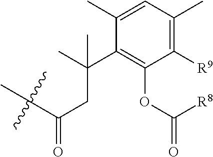

wherein R⁸ is methyl and R⁹ is H.

R¹ may be selected from the group consisting of methyl, ethyl, propyl, isopropyl, benzyl and R² may be a masking group.

R¹ may be selected from the group consisting of methyl, ethyl, propyl, isopropyl, benzyl and R² may be

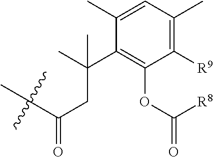

wherein R⁸ is methyl and R⁹ is H.

R⁴ may be selected from the group consisting of: H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁴ may be selected from the group consisting of: C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁴ may be selected from the group consisting of: H, C₁-C₆ alkyl, C₁-C₆ haloalkyl, halo, NO₂, CN, C(O)R^{b}, and S(O)₂R^{e}.

R⁴ may be selected from the group consisting of: C₁-C₆ alkyl, C₁-C₆ haloalkyl, halo, NO₂, CN, C(O)R^{b}, and S(O)₂R^{e}.

For example, R⁴ may be selected from the group consisting of: H, methyl, ethyl, propyl, isopropyl, butyl, tert-butyl, C₂-alkenyl, C₃-alkenyl, C₃-alkynyl, CF₃, CCl₃, F, Cl, Br, I, NO₂, CN, OCH₃, OCH₂CH₃, OH, CH₂OCH₃, CH₂OCH₂CH₃, CH₂OH, SCH₃, SCH₂CH₃, SH, CH₂SCH₃, CH₂SCH₂CH₃, CH₂SH, C(O)CH₃, C(O)NH₂, SO₂F and NH₂.

For example, R⁴ may be selected from the group consisting of: H, methyl, ethyl, propyl, isopropyl, butyl, tert-butyl, CF₃, F, Cl, Br, I, NO₂, CN, OCH₃, OH, CH₂OCH₃, C(O)CH₃, C(O)NH₂, SO₂F and NH₂.

R⁴ may be selected from the group consisting of: H, methyl, ethyl, propyl, isopropyl, butyl, tert-butyl, C₁-C₆ haloalkyl, halo, NO₂, CN, C(O)CH₃ and S(O)₂F.

R⁴ may be selected from the group consisting of: methyl, ethyl, propyl, isopropyl, butyl, tert-butyl, C₁-C₆ haloalkyl, halo, NO₂, CN, C(O)CH₃ and S(O)₂F.

R⁴ may be selected from the group consisting of: H, halo, NO₂, CF₃ and CN.

R⁴ may be selected from the group consisting of: halo, NO₂, CF₃ and CN.

R⁴ may be selected from the group consisting of: H, F, Cl, Br, I, NO₂, CF₃ and CN.

R⁴ may be selected from the group consisting of: F, Cl, Br, I, NO₂, CF₃ and CN.

R⁴ may be selected from the group consisting of: F, NO₂ and CN.

R⁴ may be F.

R⁴ may be selected from the group consisting of: H, F, NO₂ and CN.

R⁵ may be selected from the group consisting of: H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁵ may be selected from the group consisting of: C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁵ may be selected from the group consisting of: H, C₁-C₆ alkyl, C₁-C₆ haloalkyl, halo, NO₂, CN, C(O)R^{b}, and S(O)₂R^{e}.

R⁵ may be selected from the group consisting of: C₁-C₆ alkyl, C₁-C₆ haloalkyl, halo, NO₂, CN, C(O)R^{b}, and S(O)₂R^{e}.

For example, R⁵ may be selected from the group consisting of: H, methyl, ethyl, propyl, isopropyl, butyl, tert-butyl, C₂-alkenyl, C₃-alkenyl, C₃-alkynyl, CF₃, CCl₃, F, Cl, Br, I, NO₂, CN, OCH₃, OCH₂CH₃, OH, CH₂OCH₃, CH₂OCH₂CH₃, CH₂OH, SCH₃, SCH₂CH₃, SH, CH₂SCH₃, CH₂SCH₂CH₃, CH₂SH, C(O)CH₃, C(O)NH₂, SO₂F and NH₂.

For example, R⁵ may be selected from the group consisting of: H, methyl, ethyl, propyl, isopropyl, butyl, tert-butyl, CF₃, F, Cl, Br, I, NO₂, CN, OCH₃, OH, CH₂OCH₃, C(O)CH₃, C(O)NH₂, SO₂F and NH₂.

R⁵ may be selected from the group consisting of: H, methyl, ethyl, propyl, isopropyl, butyl, tert-butyl, C₁-C₆ haloalkyl, halo, NO₂, CN, C(O)CH₃ and S(O)₂F.

R⁵ may be selected from the group consisting of: methyl, ethyl, propyl, isopropyl, butyl, tert-butyl, C₁-C₆ haloalkyl, halo, NO₂, CN, C(O)CH₃ and S(O)₂F.

R⁵ may be selected from the group consisting of: H, halo, NO₂, CF₃ and CN.

R⁵ may be selected from the group consisting of: halo, NO₂, CF₃ and CN.

R⁵ may be selected from the group consisting of: H, F, Cl, Br, I, NO₂, CF₃ and CN.

R⁵ may be selected from the group consisting of: F, Cl, Br, I, NO₂, CF₃ and CN.

R⁵ may be selected from the group consisting of: F, NO₂ and CN.

R⁵ may be selected from the group consisting of: H, F, NO₂ and CN.

R⁵ may be H.

R⁶ may be selected from the group consisting of: H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁶ may be selected from the group consisting of: C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁶ may be selected from the group consisting of: H, C₁-C₆ alkyl, C₁-C₆ haloalkyl, halo, NO₂, CN, C(O)R^{b}, and S(O)₂R^{e}.

R⁶ may be selected from the group consisting of: C₁-C₆ alkyl, C₁-C₆ haloalkyl, halo, NO₂, CN, C(O)R^{b}, and S(O)₂R^{e}.

For example, R⁶ may be selected from the group consisting of: H, methyl, ethyl, propyl, isopropyl, butyl, tert-butyl, C₂-alkenyl, C₃-alkenyl, C₃-alkynyl, CF₃, CCl₃, F, Cl, Br, I, NO₂, CN, OCH₃, OCH₂CH₃, OH, CH₂OCH₃, CH₂OCH₂CH₃, CH₂OH, SCH₃, SCH₂CH₃, SH, CH₂SCH₃, CH₂SCH₂CH₃, CH₂SH, C(O)CH₃, C(O)NH₂, SO₂F and NH₂.

For example, R⁶ may be selected from the group consisting of: H, methyl, ethyl, propyl, isopropyl, butyl, tert-butyl, CF₃, F, Cl, Br, I, NO₂, CN, OCH₃, OH, CH₂OCH₃, C(O)CH₃, C(O)NH₂, SO₂F and NH₂.

R⁶ may be selected from the group consisting of: H, methyl, ethyl, propyl, isopropyl, butyl, tert-butyl, C₁-C₆ haloalkyl, halo, NO₂, CN, C(O)CH₃ and S(O)₂F.

R⁶ may be selected from the group consisting of: methyl, ethyl, propyl, isopropyl, butyl, tert-butyl, C₁-C₆ haloalkyl, halo, NO₂, CN, C(O)CH₃ and S(O)₂F.

R⁶ may be selected from the group consisting of: H, halo, NO₂, CF₃ and CN.

R⁶ may be selected from the group consisting of: halo, NO₂, CF₃ and CN.

R⁶ may be selected from the group consisting of: H, F, Cl, Br, I, NO₂, CF₃ and CN.

R⁶ may be selected from the group consisting of: F, Cl, Br, I, NO₂, CF₃ and CN.

R⁶ may be selected from the group consisting of: F, NO₂ and CN.

R⁶ may be selected from the group consisting of: H, F, NO₂ and CN.

R⁶ may be H. R⁵ may be H and R⁶ may be H.

R⁷ may be selected from the group consisting of: H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁷ may be selected from the group consisting of: C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, S(O)₂R^{e} and NR^{f}R^{g}.

R⁷ may be selected from the group consisting of: H, C₁-C₆ alkyl, C₁-C₆ haloalkyl, halo, NO₂, CN, C(O)R^{b}, and S(O)₂R^{e}.

R⁷ may be selected from the group consisting of: C₁-C₆ alkyl, C₁-C₆ haloalkyl, halo, NO₂, CN, C(O)R^{b}, and S(O)₂R^{e}.

For example, R⁷ may be selected from the group consisting of: H, methyl, ethyl, propyl, isopropyl, butyl, tert-butyl, C₂-alkenyl, C₃-alkenyl, C₃-alkynyl, CF₃, CCl₃, F, Cl, Br, I, NO₂, CN, OCH₃, OCH₂CH₃, OH, CH₂OCH₃, CH₂OCH₂CH₃, CH₂OH, SCH₃, SCH₂CH₃, SH, CH₂SCH₃, CH₂SCH₂CH₃, CH₂SH, C(O)CH₃, C(O)NH₂, SO₂F and NH₂.

For example, R⁷ may be selected from the group consisting of: H, methyl, ethyl, propyl, isopropyl, butyl, tert-butyl, CF₃, F, Cl, Br, I, NO₂, CN, OCH₃, OH, CH₂OCH₃, C(O)CH₃, C(O)NH₂, SO₂F and NH₂.

R⁷ may be selected from the group consisting of: H, methyl, ethyl, propyl, isopropyl, butyl, tert-butyl, C₁-C₆ haloalkyl, halo, NO₂, CN, C(O)CH₃ and S(O)₂F.

R⁷ may be selected from the group consisting of: methyl, ethyl, propyl, isopropyl, butyl, tert-butyl, C₁-C₆ haloalkyl, halo, NO₂, CN, C(O)CH₃ and S(O)₂F.

R⁷ may be selected from the group consisting of: H, halo, NO₂, CF₃ and CN.

R⁷ may be selected from the group consisting of: halo, NO₂, CF₃ and CN.

R⁷ may be selected from the group consisting of: H, F, Cl, Br, I, NO₂, CF₃ and CN.

R⁷ may be selected from the group consisting of: F, Cl, Br, I, NO₂, CF₃ and CN.

R⁷ may be selected from the group consisting of: F, NO₂ and CN.

R⁷ may be F.

R⁷ may be selected from the group consisting of: H, F, NO₂ and CN.

R⁴, R⁵, R⁶ and R⁷ may be each independently selected from H, haloalkyl, C₁-C₆ alkyl, halo, NO₂, CN, C(O)R^{b}, SO₂R^{e} and C(O)NR^{c}R^{d}.

R⁴, R⁵, R⁶ and R⁷ may be each independently selected from H, CF₃, F, Cl, Br, I, NO₂, CN, C(O)CH₃ and C(O)NH₂.

R⁴, R⁵, R⁶ and R⁷ may be each independently selected from H, CF₃, F, Cl, Br, I, NO₂, CN and C(O)CH₃.

X′ may be NH₂ and R⁴, R⁵, R⁶ and R⁷ may be each independently selected from H, CF₃, F, Cl, Br, I, NO₂, CN and C(O)CH₃.

X′ may be Cl and R⁴, R⁵, R⁶ and R⁷ may be each independently selected from H, CF₃, F, Cl, Br, I, NO₂, CN and C(O)CH₃. Each of R⁴ and R⁷ may be independently selected from H, halo, NO₂, CN and C(O)NR^{c}R^{d}; R⁵ is H; and R⁶ is H.

Each of R⁴ and R⁷ may be independently selected from H, halo, NO₂, CN and C(O)NH₂; R⁵ is H; and R⁶ is H.

Each of R⁴ and R⁷ may be independently selected from H, F, Cl, Br, I, NO₂, CN and C(O)NH₂; R⁵ is H; and R⁶ is H.

Both of R⁴ and R⁷ may be F, and both of R⁵ and R⁶ may be H.

Each of R⁴, R⁵, R⁶ and R⁷ may be halo (optionally F), or H.

Each of R⁴, R⁵, R⁶ and R⁷ may be H.

Each of R⁴, R⁵, R⁶ and R⁷ may be F.

R⁴, R⁵, R⁶ and R⁷ may be each be F and R³ may be CN.

R⁴ may be F and R⁷ may be F. R⁴ may be F, R⁷ may be F and R³ may be CN.

R⁴ may be F, R⁷ may be F, R³ may be CN, R⁵ may be H and R⁶ may be H.

R³ may be an electron withdrawing group and R⁴, R⁵, R⁶ and R⁷ may be each independently selected from H, haloalkyl, halo, NO₂, CN and C(O)NR^{c}R^{d}. R³ may be CN and R⁴, R⁵, R⁶ and R⁷ may be each independently selected from H, haloalkyl, halo, NO₂, CN and C(O)NR^{c}R^{d}.

R³ may be an electron withdrawing group and each of R⁴ and R⁷ may be independently selected from H, halo, NO₂, CN and C(O)NR^{c}R^{d}; R⁵ is H; and R⁶ is H. R³ may be CN and each of R⁴ and R⁷ may be independently selected from H, halo, NO₂, CN and C(O)NR^{c}R^{d}; R⁵ is H; and R⁶ is H.

R³ may be an electron withdrawing group and each of R⁴ and R⁷ may be independently selected from H, halo, NO₂, CN and C(O)NH₂; R⁵ is H; and R⁶ is H. R³ may be CN and each of R⁴ and R⁷ may be independently selected from H, halo, NO₂, CN and C(O)NH₂; R⁵ is H; and R⁶ is H.

R³ may be an electron withdrawing group and each of R⁴, R⁵, R⁶ and R⁷ may be F, Cl, Br, I or H. R³ may be CN and each of R⁴, R⁵, R⁶ and R⁷ may be F, Cl, Br, I or H.

R³ may be an electron withdrawing group and each of R⁴, R⁵, R⁶ and R⁷ may be halo (optionally F), or H. R³ may be CN and each of R⁴, R⁵, R⁶ and R⁷ may be halo (optionally F), or H.

R³ may be an electron withdrawing group and each of R⁴, R⁵, R⁶ and R⁷ may be H. R³ may be CN and each of R⁴, R⁵, R⁶ and R⁷ may be H.

R³ may be an electron withdrawing group and each of R⁴, R⁵, R⁶ and R⁷ may be F. R³ may be CN and each of R⁴, R⁵, R⁶ and R⁷ may be F.

R³ may be CN, R⁵ may be H, R⁶ may be H, R⁴ may be F and R⁷ may be F.

R^{a}, R^{b}, R^{c}, R^{d}, R^{e}, R^{f} and R^{g} may be each independently at each occurrence selected from the group consisting of: H, F, Cl, Br, I, C₁-C₃ alkyl, C₂-C₅ alkenyl, and C₁-C₃ haloalkyl.

R^{a}, R^{b}, R^{c}, R^{d}, R^{e}, R^{f} and R^{g} may be each independently at each occurrence selected from the group consisting of: H, F, Cl, Br, I, C₁-C₃ alkyl, and C₁-C₃ haloalkyl.

R^{a}, R^{b}, R^{c}, R^{d}, R^{e}, R^{f} and R^{g} may be each independently at each occurrence selected from the group consisting of: H, F, Cl, Br, I, methyl, ethyl, propyl and isopropyl.

R^{a}, R^{b}, R^{c}, R^{d}, R^{e}, R^{f} and R^{g} may be each independently at each occurrence H.

The compound may be a compound selected from:
a compound of formula III,

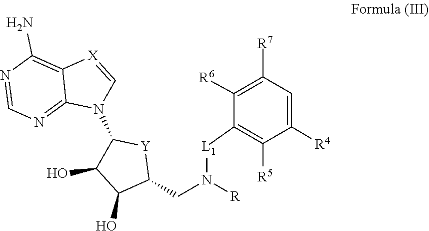

wherein:
X is selected from N and CH;
Y is selected from O and CH₂;
L₁ is selected from the group consisting of: substituted or unsubstituted C₂-C₅ alkyl, substituted or unsubstituted C₂-C₅ alkenyl and substituted or unsubstituted C₂-C₅ alkynyl;
R is selected from the group consisting of:

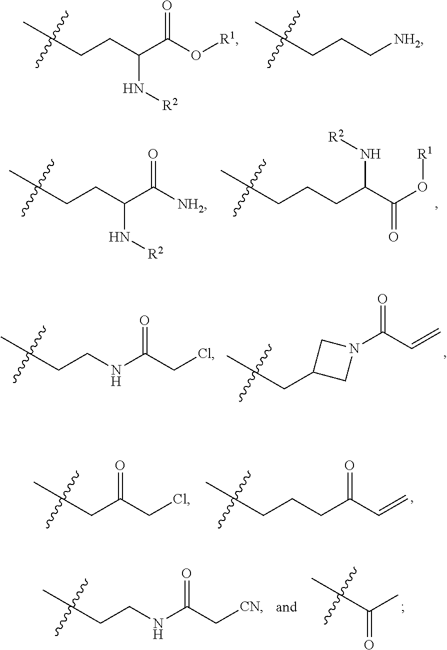

R¹ is selected from the group consisting of: H and a masking group;
R² is selected from the group consisting of: H and a masking group;
R⁴ is selected from the group consisting of: C₁-C₆ haloalkyl, halo, NO₂, CN, OR^{a}, CH₂OR^{a}, SR^{a}, CH₂SR^{a}, C(O)OR^{b}, C(O)NR^{c}R^{d}, S(O)₂R^{e};
R⁵, R⁶ and R⁷ are each independently selected from H, C₁-C₆ alkyl, C₂-C₆ alkenyl, C₂-C₆ alkynyl, C₁-C₆ haloalkyl, halo, NO₂, CN, B(OH)₂, OR^{a}, CH₂OR^{a}, SR^{a}, CH₂SR^{a}, C(O)OR^{b}, C(O)NR^{c}R^{d}, S(O)₂R^{e} and NR^{f}R^{g};
R^{a}, R^{b}, R^{c}, R^{d}, R^{e}, R^{f} and R^{g} are each independently at each occurrence selected from the group consisting of: H, halo, C₁-C₃ alkyl and C₁-C₃ haloalkyl;
or a pharmaceutically acceptable salt, stereoisomer, solvate or prodrug thereof.

The compound may be a compound selected from a compound of formula IIIA:

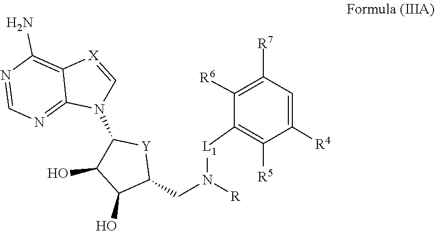

wherein:
X is selected from N and CH;
Y is selected from O and CH₂;
L₁ is selected from the group consisting of: substituted or unsubstituted C₂-C₅ alkyl, substituted or unsubstituted C₂-C₅ alkenyl and substituted or unsubstituted C₂-C₅ alkynyl;
R is selected from the group consisting of:

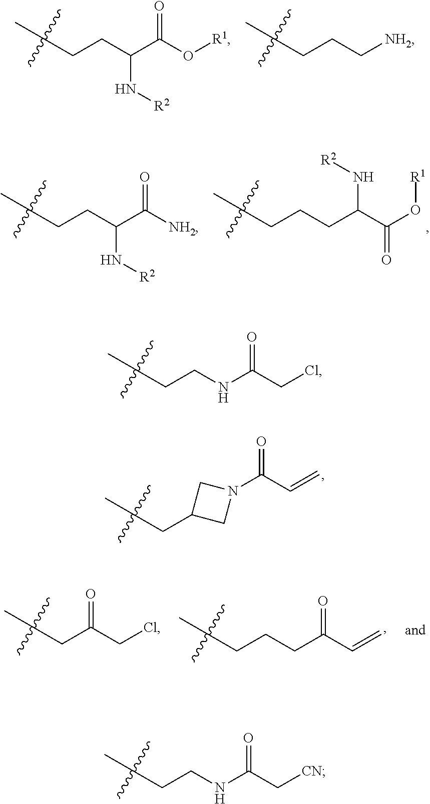

R¹ is selected from the group consisting of: H and a masking group;
R² is selected from the group consisting of: H and a masking group;
R⁴ is selected from the group consisting of: C₁-C₆ haloalkyl, halo, NO₂, CN, OR^{a}, CH₂OR^{a}, SR^{a}, CH₂SR^{a}, C(O)OR^{b}, C(O)NR^{c}R^{d}, S(O)₂R^{e};
R⁵, R⁶ and R⁷ are each independently selected from H, C₁-C₆ alkyl, C₂-C₆ alkenyl, C₂-C₆ alkynyl, C₁-C₆ haloalkyl, halo, NO₂, CN, B(OH)₂, OR^{a}, CH₂OR^{a}, SR^{a}, CH₂SR^{a}, C(O)OR^{b}, C(O)NR^{c}R^{d}, S(O)₂R^{e} and NR^{f}R^{g};
R^{a}, R^{b}, R^{c}, R^{d}, R^{e}, R^{f} and R^{g} are each independently at each occurrence selected from the group consisting of: H, halo, C₁-C₃ alkyl and C₁-C₃ haloalkyl;
or a pharmaceutically acceptable salt, stereoisomer, solvate or prodrug thereof.

R⁴ may be selected from the group consisting of: C₁-C₆ alkyl, C₁-C₆ haloalkyl, halo, NO₂, CN, C(O)R^{b}, C(O)NR^{c}R^{d}, and S(O)₂R^{e}. R⁴ may be selected from the group consisting of: C₁-C₆ alkyl, C₁-C₆ haloalkyl, halo, NO₂, CN, C(O)R^{b}, C(O)NR^{c}R^{d}, and S(O)₂R^{e}. R⁴ may be selected from the group consisting of: halo, NO₂, CF₃, C(O)NR^{c}R^{d}, and CN. R⁴ may be selected from the group consisting of: F, Cl, Br, I, NO₂, CF₃ and CN. R⁴ may be selected from the group consisting of: F, NO₂, C(O)NR^{c}R^{d}, and CN. R⁴ may be C(O)NR^{c}R^{d}.

When R⁴ is C(O)NR^{c}R^{d}, R^{C} and R^{d} may each independently be selected from the group consisting of: H and C₁-C₃ alkyl; e.g. R^{c} and R^{d} may each be H.

X, Y, L₁, R, R¹, R², R⁵, R⁶ and R⁷ may be as defined elsewhere herein.

In an embodiment, the compound (for example a compound of any of formula I, II or III) is subject to the proviso that the compound is not a compound of formula IV:

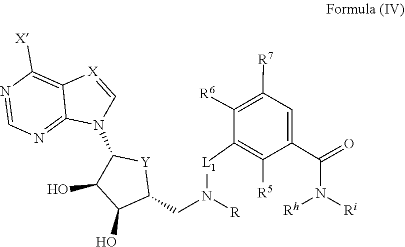

wherein:
L₁ is selected from the group consisting of: C₂-C₅ alkyl and C₂-C₅ alkynyl;
R^{h} and Rⁱ are each independently at each occurrence selected from the group consisting of: H, halo, C₁-C₃ alkyl and C₁-C₃ haloalkyl; optionally wherein R^{h} and Rⁱ are both H;
or a pharmaceutically acceptable salt, stereoisomer, solvate or prodrug thereof.

In an embodiment, the compound (for example a compound of any of formula I, II or III) is subject to the proviso that the compound is not a compound of formula IVA:

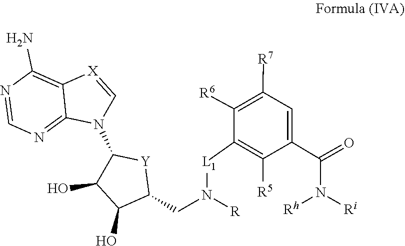

wherein:
L₁ is selected from the group consisting of: C₂-C₅ alkyl and C₂-C₅ alkynyl;
R^{h} and Rⁱ are each independently at each occurrence selected from the group consisting of: H, halo, C₁-C₃ alkyl and C₁-C₃ haloalkyl; optionally wherein R^{h} and Rⁱ are both H;
or a pharmaceutically acceptable salt, stereoisomer, solvate or prodrug thereof.

In an embodiment, the compound of the invention is subject to the proviso that the compound is not:

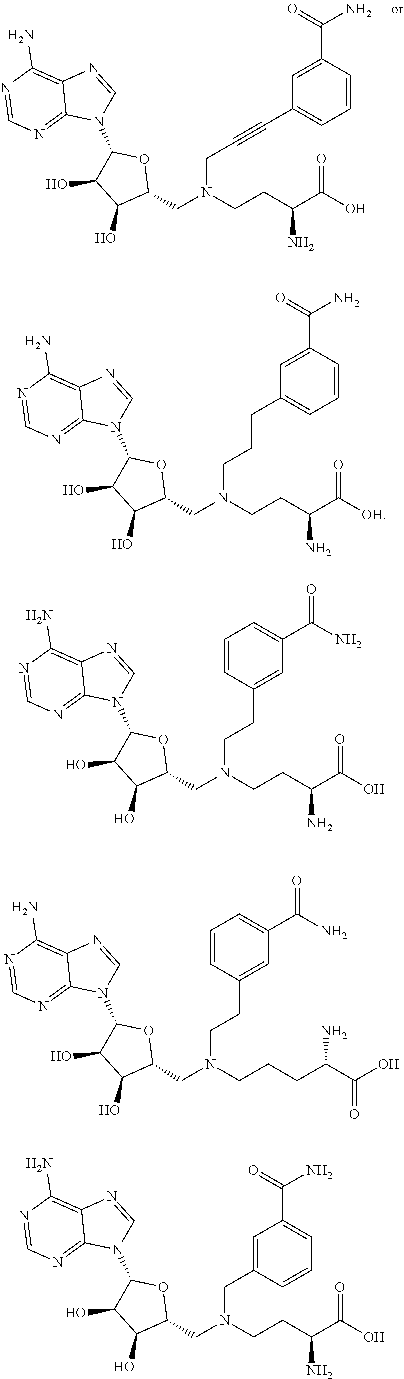

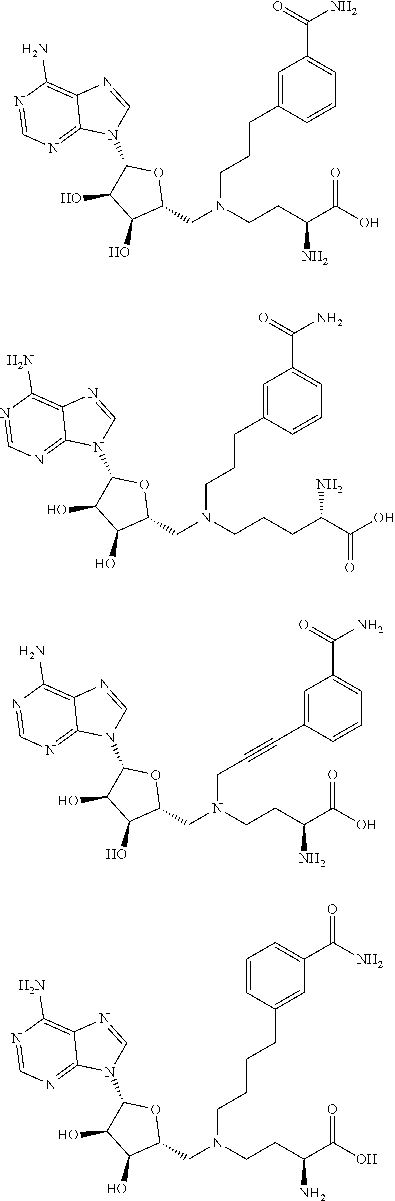 [SMILES: NC(=O)c1cccc(CCCN(CC[C@H](N)C(=O)O)C[C@H]2O[C@@H](n3cnc4c(N)ncnc43)[C@H](O)[C@@H]2O)c1 ; NC(=O)c1cccc(CCCN(CCC[C@H](N)C(=O)O)C[C@H]2O[C@@H](n3cnc4c(N)ncnc43)[C@H](O)[C@@H]2O)c1 ; NC(=O)c1cccc(C#CCN(CC[C@H](N)C(=O)O)C[C@H]2O[C@@H](n3cnc4c(N)ncnc43)[C@H](O)[C@@H]2O)c1 ; NC(=O)c1cccc(CCCCN(CC[C@H](N)C(=O)O)C[C@H]2O[C@@H](n3cnc4c(N)ncnc43)[C@H](O)[C@@H]2O)c1]

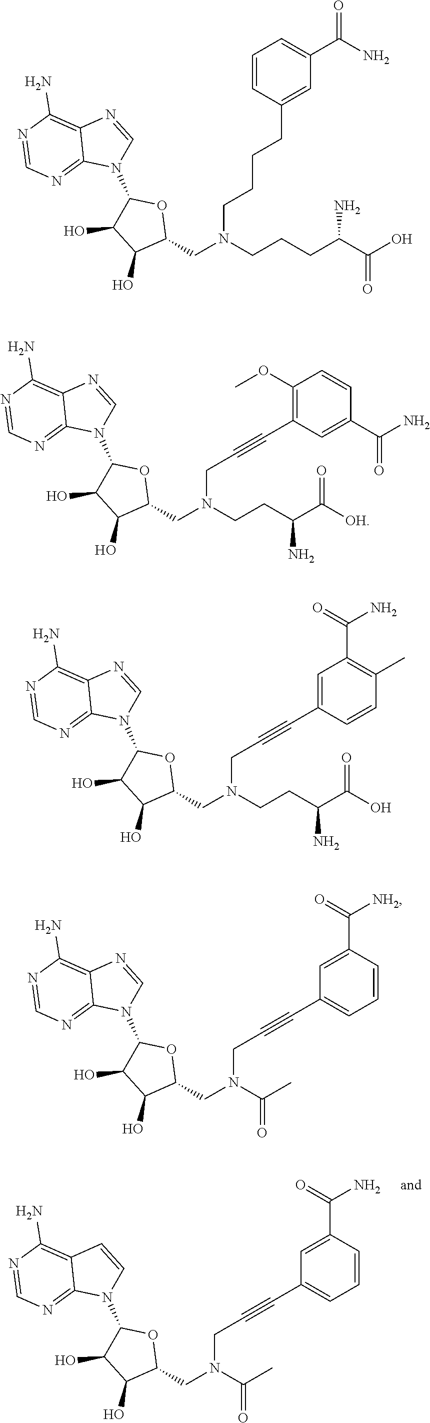 [SMILES: NC(=O)c1cccc(CCCCN(CCC[C@H](N)C(=O)O)C[C@H]2O[C@@H](n3cnc4c(N)ncnc43)[C@H](O)[C@@H]2O)c1 ; COc1ccc(C(N)=O)cc1C#CCN(CC[C@H](N)C(=O)O)C[C@H]1O[C@@H](n2cnc3c(N)ncnc32)[C@H](O)[C@@H]1O ; Cc1ccc(C#CCN(CC[C@H](N)C(=O)O)C[C@H]2O[C@@H](n3cnc4c(N)ncnc43)[C@H](O)[C@@H]2O)cc1C(N)=O ; CC(=O)N(CC#Cc1cccc(C(N)=O)c1)C[C@H]1O[C@@H](n2cnc3c(N)ncnc32)[C@H](O)[C@@H]1O ; CC(=O)N(CC#Cc1cccc(C(N)=O)c1)C[C@H]1O[C@@H](n2ccc3c(N)ncnc32)[C@H](O)[C@@H]1O]

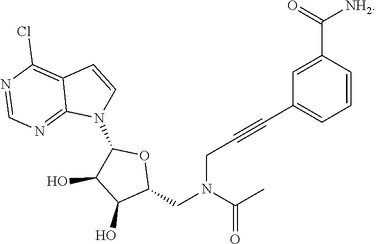 [SMILES: CC(=O)N(CC#Cc1cccc(C(N)=O)c1)C[C@H]1O[C@@H](n2ccc3c(Cl)ncnc32)[C@H](O)[C@@H]1O]

In an embodiment, the compound is selected from:

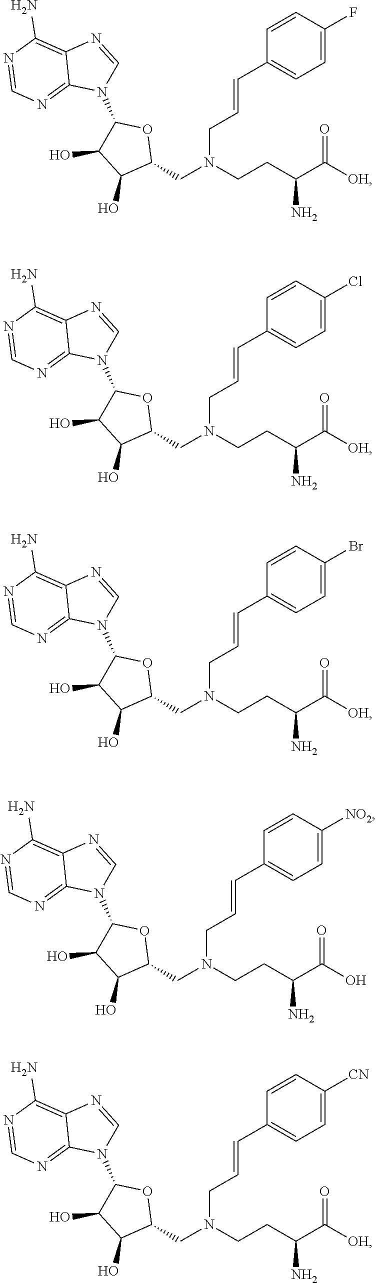 [SMILES: Nc1ncnc2c1ncn2[C@@H]1O[C@H](CN(C/C=C/c2ccc(F)cc2)CC[C@H](N)C(=O)O)[C@@H](O)[C@H]1O ; Nc1ncnc2c1ncn2[C@@H]1O[C@H](CN(C/C=C/c2ccc(Cl)cc2)CC[C@H](N)C(=O)O)[C@@H](O)[C@H]1O ; Nc1ncnc2c1ncn2[C@@H]1O[C@H](CN(C/C=C/c2ccc(Br)cc2)CC[C@H](N)C(=O)O)[C@@H](O)[C@H]1O ; Nc1ncnc2c1ncn2[C@@H]1O[C@H](CN(C/C=C/c2ccc([N+](=O)[O-])cc2)CC[C@H](N)C(=O)O)[C@@H](O)[C@H]1O ; N#Cc1ccc(/C=C/CN(CC[C@H](N)C(=O)O)C[C@H]2O[C@@H](n3cnc4c(N)ncnc43)[C@H](O)[C@@H]2O)cc1]

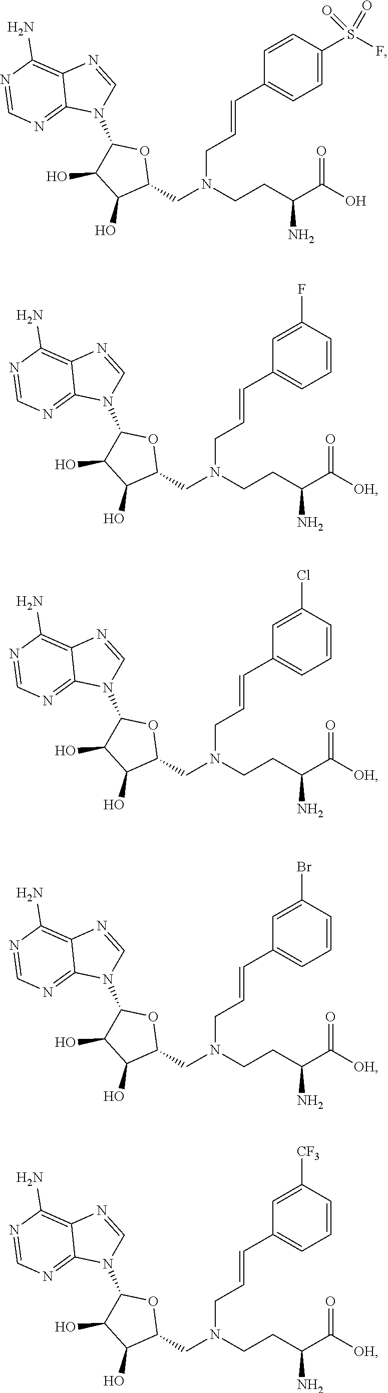 [SMILES: Nc1ncnc2c1ncn2[C@@H]1O[C@H](CN(C/C=C/c2ccc(S(=O)(=O)F)cc2)CC[C@H](N)C(=O)O)[C@@H](O)[C@H]1O ; Nc1ncnc2c1ncn2[C@@H]1O[C@H](CN(C/C=C/c2cccc(F)c2)CC[C@H](N)C(=O)O)[C@@H](O)[C@H]1O ; Nc1ncnc2c1ncn2[C@@H]1O[C@H](CN(C/C=C/c2cccc(Cl)c2)CC[C@H](N)C(=O)O)[C@@H](O)[C@H]1O ; Nc1ncnc2c1ncn2[C@@H]1O[C@H](CN(C/C=C/c2cccc(Br)c2)CC[C@H](N)C(=O)O)[C@@H](O)[C@H]1O ; Nc1ncnc2c1ncn2[C@@H]1O[C@H](CN(C/C=C/c2cccc(C(F)(F)F)c2)CC[C@H](N)C(=O)O)[C@@H](O)[C@H]1O]

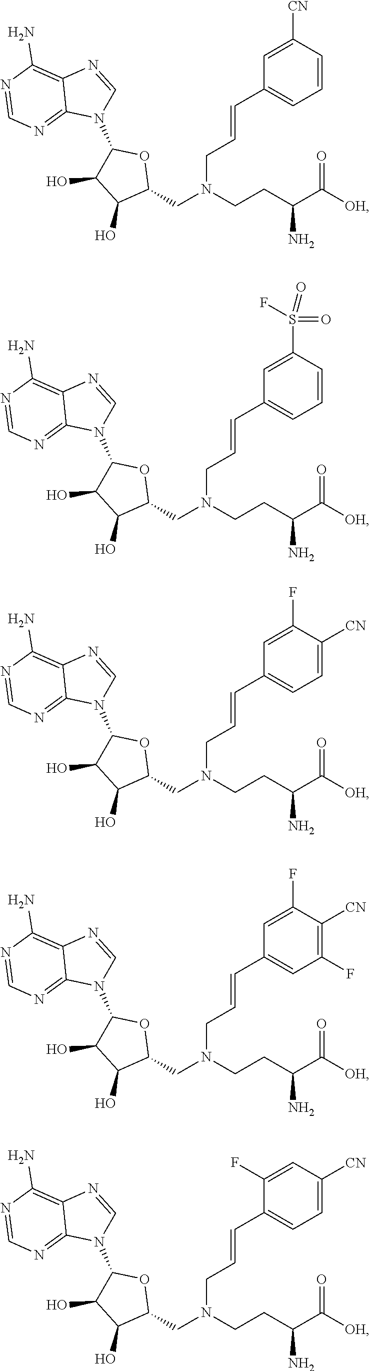

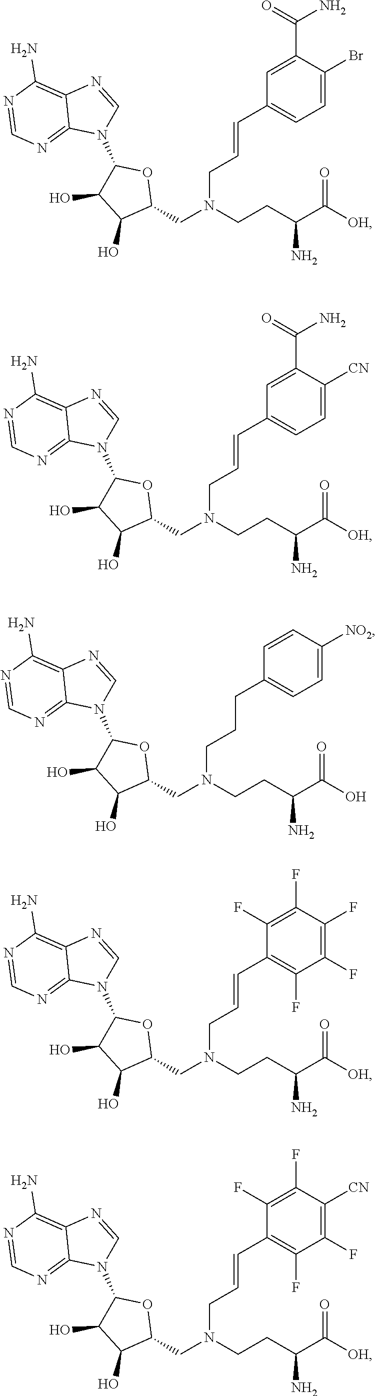

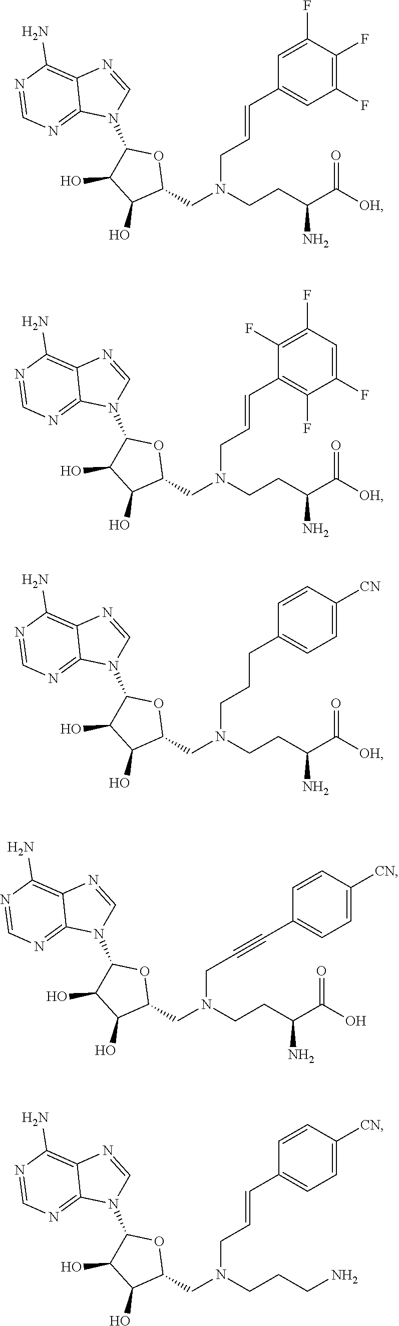 [SMILES: Nc1ncnc2c1ncn2[C@@H]1O[C@H](CN(C/C=C/c2cc(F)c(F)c(F)c2)CC[C@H](N)C(=O)O)[C@@H](O)[C@H]1O ; Nc1ncnc2c1ncn2[C@@H]1O[C@H](CN(C/C=C/c2c(F)c(F)cc(F)c2F)CC[C@H](N)C(=O)O)[C@@H](O)[C@H]1O ; N#Cc1ccc(CCCN(CC[C@H](N)C(=O)O)C[C@H]2O[C@@H](n3cnc4c(N)ncnc43)[C@H](O)[C@@H]2O)cc1 ; N#Cc1ccc(C#CCN(CC[C@H](N)C(=O)O)C[C@H]2O[C@@H](n3cnc4c(N)ncnc43)[C@H](O)[C@@H]2O)cc1 ; N#Cc1ccc(/C=C/CN(CCCN)C[C@H]2O[C@@H](n3cnc4c(N)ncnc43)[C@H](O)[C@@H]2O)cc1]

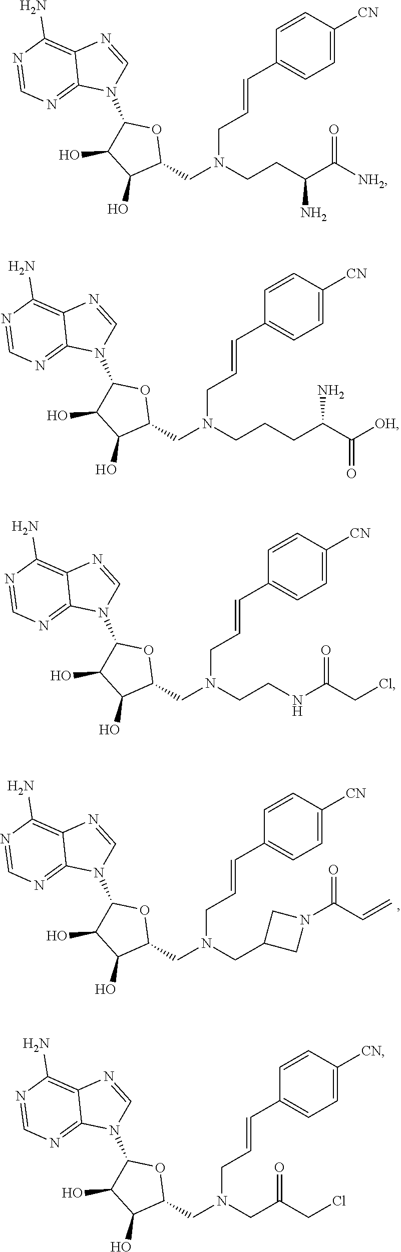 [SMILES: N#Cc1ccc(/C=C/CN(CC[C@H](N)C(N)=O)C[C@H]2O[C@@H](n3cnc4c(N)ncnc43)[C@H](O)[C@@H]2O)cc1 ; N#Cc1ccc(/C=C/CN(CCC[C@H](N)C(=O)O)C[C@H]2O[C@@H](n3cnc4c(N)ncnc43)[C@H](O)[C@@H]2O)cc1 ; N#Cc1ccc(/C=C/CN(CCNC(=O)CCl)C[C@H]2O[C@@H](n3cnc4c(N)ncnc43)[C@H](O)[C@@H]2O)cc1 ; C=CC(=O)N1CC(CN(C/C=C/c2ccc(C#N)cc2)C[C@H]2O[C@@H](n3cnc4c(N)ncnc43)[C@H](O)[C@@H]2O)C1 ; N#Cc1ccc(/C=C/CN(CC(=O)CCl)C[C@H]2O[C@@H](n3cnc4c(N)ncnc43)[C@H](O)[C@@H]2O)cc1]

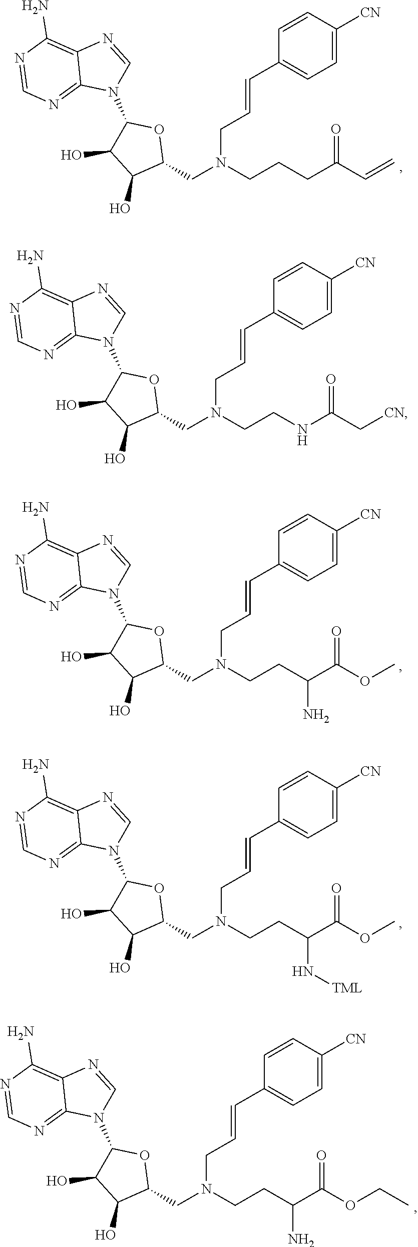

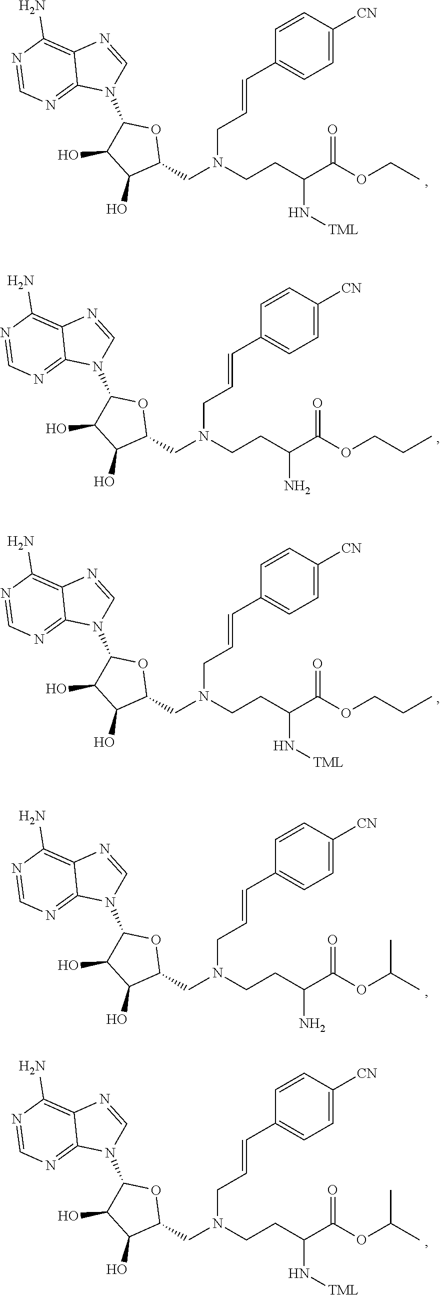

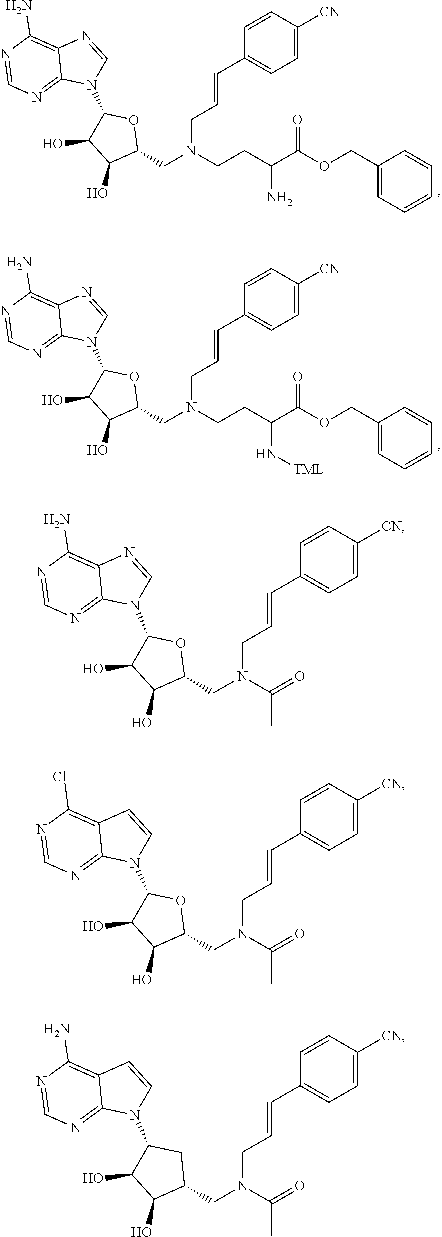

where TML is selected from

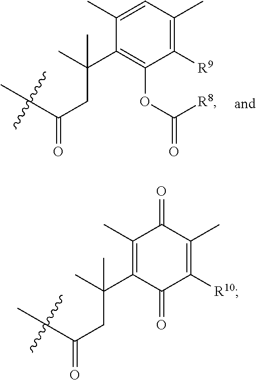

or a pharmaceutically acceptable salt, stereoisomer, or solvate thereof.

Exemplary compounds may be made in accordance with the methods of synthesis illustrated in the examples. In addition, as the skilled person will appreciate, these methods and the methods illustrated in the reaction schemes 1 to 8 may be readily adapted to provide other compounds of the present disclosure.

### Protac Compounds

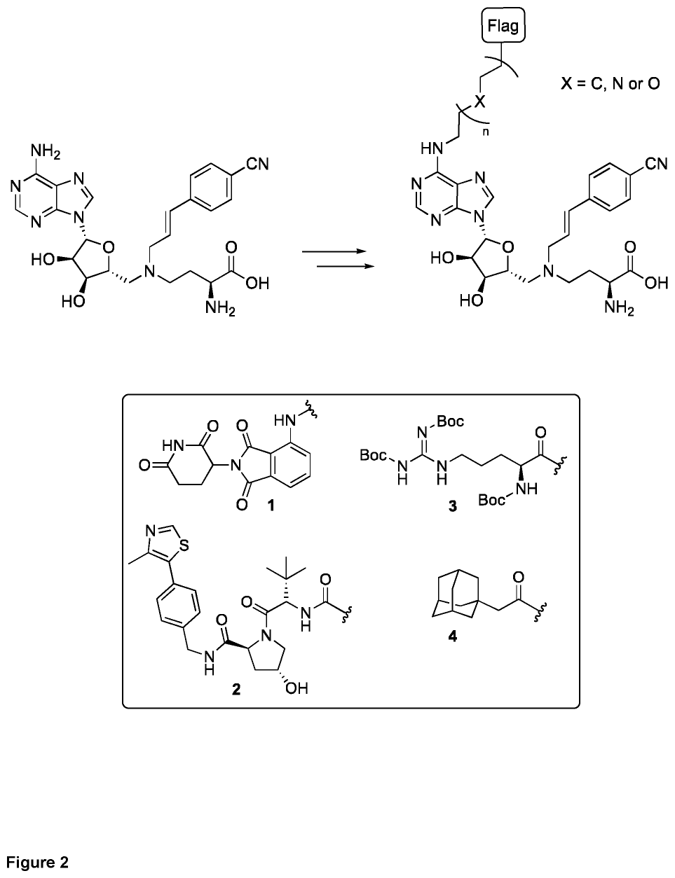

Potent ligands can be used for the development of targeted protein degraders, e.g. through inclusion of hydrophobic tags or the synthesis of proteolysis targeting chimeras (PROTACs, see FIG. 2) (Toure M, Crews C M. Small-molecule PROTACS: New approaches to protein degradation. Angew Chemie—Int Ed 2016; 55: 1966-73). In hydrophobic tagging, a bulky and hydrophobic group is linked to a small-molecule binder of the target protein. Upon binding to the target, the hydrophobic tag causes misfolding of the target protein resulting in degradation by the proteasome. Exemplary hydrophobic tags are provided by groups 3 and 4 illustrated in FIG. 2. A similar but less direct approach to targeted degradation is the use of PROTACs. In this technique a ligand for a targeted protein is linked to a ligand for a selected E3 ligase. This bifunctional molecule will bring the targeted protein in close proximity to an E3 ligase, thereby facilitating the E3-ligase-mediated poly-ubiquitination of the targeted protein.

This polyubiquitin modification flags the protein for degradation by the proteasome. Exemplary E3-ligases ligands are provided by groups 1 and 2 illustrated in FIG. 2.

Exemplary PROTAC compounds of the invention may have formula Iα, IIα, IIAα, IIBα, IICα or IIIα:

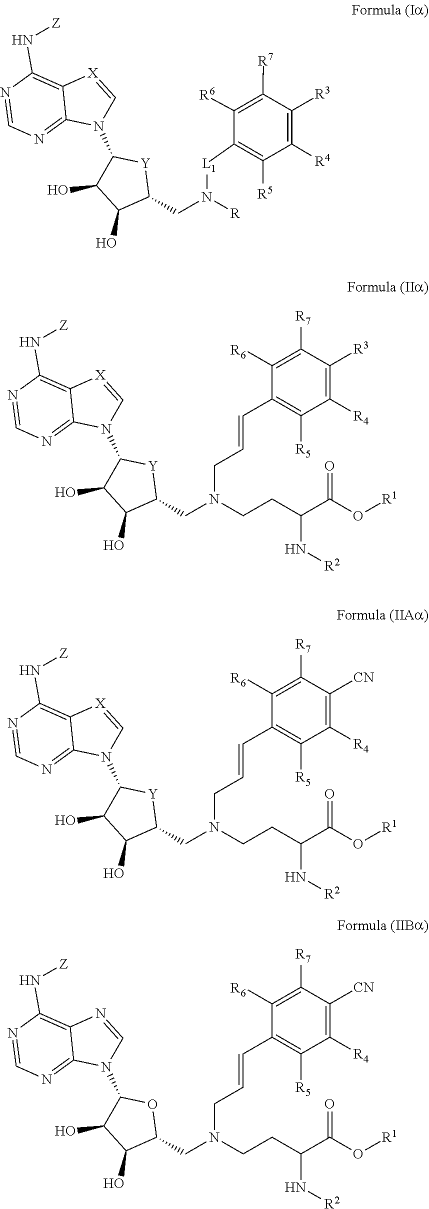

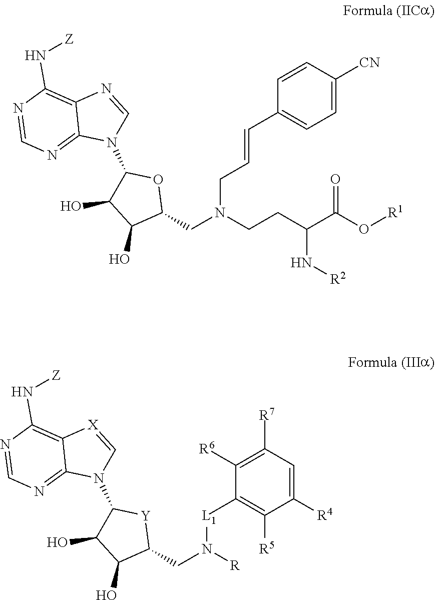

or a pharmaceutically acceptable salt, stereoisomer, solvate or prodrug thereof.
   X, Y, L₁, R, R¹, R², R³, R⁴, R⁵, R⁶, R⁷ are as defined elsewhere for formulae I, II, IIA, IIB, IIC, or III.

### Z is

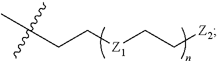

where n is an integer from 1 to 20; Z₁ is C, N or O; and Z₂ is an E3-ligase ligand or a hydrophobic tag. Z₂ may be selected from:

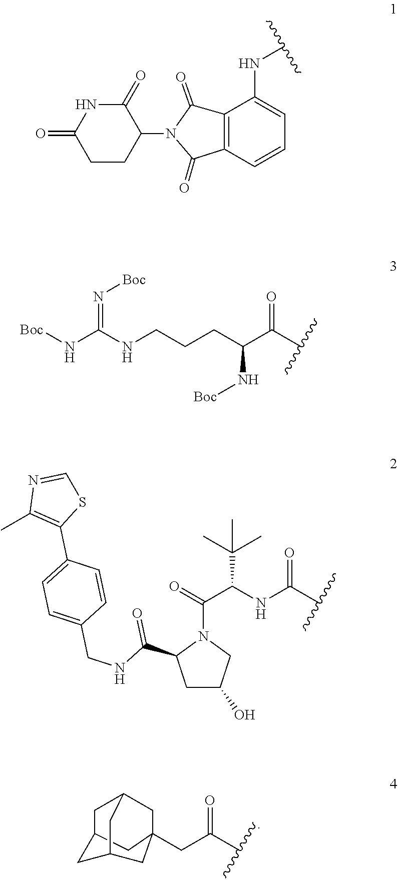 [SMILES: *Nc1cccc2c1C(=O)N(C1CCC(=O)NC1=O)C2=O ; *C(=O)[C@H](CCCN/C(=N\C(=O)OC(C)(C)C)NC(=O)OC(C)(C)C)NC(=O)OC(C)(C)C ; *C(=O)N[C@H](C(=O)N1C[C@H](O)C[C@H]1C(=O)NCc1ccc(-c2scnc2C)cc1)C(C)(C)C ; *C(=O)CC12CC3CC(CC(C3)C1)C2]

### Uses

Compounds and formulations of the invention are accordingly useful as medicaments. For example, a compound or formulation of the invention may be provided for use as a medicament.

NNMT overexpression drives chemotherapy resistance in a variety of cancers. Increased NNMT activity has been connected to lung, bladder, breast, renal, oral, skin, colorectal, gastric, hepatocellular, ovarian, pancreatic, and prostate cancer, as well as glioblastoma (see, for example, Pissios P. Nicotinamide N-Methyltransferase: More Than a Vitamin B3 Clearance Enzyme. Trends Endocrinol Metab 2017; 28: 340-53; Ramsden D B, et al. Nicotinamide N-Methyltransferase in Health and Cancer. Int J Tryptophan Res 2017; 10: 117864691769173; Lu X M, Long H. Nicotinamide N-methyltransferase as a potential marker for cancer. Neoplasma 2018; 65: 656-63; and Ganzetti G, et al. Nicotinamide N-methyltransferase: potential involvement in cutaneous malignant melanoma. Melanoma Res 2018; 28: 82-88).

Overexpression of NNMT has been associated with tumor aggressiveness and shown to promote the migration, invasion, proliferation, and survival of cancer cells. At the cellular level, overexpression of NNMT facilitates epigenetic modifications by generating a metabolic methylation sink that boosts protumorigenic gene products. This finding was further substantiated by a recent proteomics-based study revealing NNMT to be a master metabolic regulator of cancer-associated fibroblasts (CAFs) (Eckert M A, et al. Proteomics reveals NNMT as a master metabolic regulator of cancer-associated fibroblasts. Nature 2019; 569: 723-8). Expression of NNMT in CAFs leads to SAM depletion and decreases histone methylation levels, resulting in extensive gene expression changes in the tumor stroma, promoting cancer metastasis. A recent investigation also found that increases in MNA levels in the tumor microenvironment lead to the inhibition of T-cell functions resulting in their decreased killing capacity and increased tumor growth (Kilgour M K, et al. 1-Methylnicotinamide is an immune regulatory metabolite in human ovarian cancer. Sci Adv 2021; 7: eabe1174). NNMT also interacts with oncogenic kinases, activated transducers and activators of transcription, and interleukins. Inhibition or down-regulation of NNMT suppresses cell proliferation, reduces tumorigenicity in mice, and causes tumor cell death via intrinsic apoptotic pathways, highlighting the potential of NNMT inhibitors as therapeutic agents.

The NNMT inhibitors disclosed herein may therefore be useful in the treatment of cancer. In addition, co-administration of an NNMT inhibitor with a chemotherapeutic agent may reduce the resistance of a cancer to the other therapeutic agent.

A compound or formulation of the invention may be used in the treatment of cancer, optionally wherein the cancer is selected from lung cancer, bladder cancer, breast cancer, colorectal cancer, gastric cancer, hepatocellular cancer, ovarian cancer, pancreatic cancer, prostate cancer, oral cancer, glioma, lymphoma, and insulinoma. The treatment may further comprise the administration of another active agent. In this context, the other active agent may itself be a prodrug (i.e. a compound transformed in vivo to the parent compound or other active compound), or the other active agent may be active in the form it is administered. The other active agent may be a chemotherapeutic agent or analogue thereof. Where the treatment comprises the administration of another active agent, the treatment may be by combined, sequential or separate administration of the present compound or formulation and the other active agent. For example, the treatment may comprise combined administration of the present compound and the other active agent. For example, the treatment may comprise administration of the present compound followed by administration of the other active agent.

Serum MNA levels have been found to be positively correlated with obesity and diabetes (Liu M, et al. Serum N1-methylnicotinamide is associated with obesity and diabetes in Chinese. J Clin Endocrinol Metab 2015; 100: 3112-7). In line with these findings, NNMT knockdowns in mice were found to be protective against diet-induced obesity via increased energy expenditure. In addition, glucose levels in NNMT-knockdown mice were significantly reduced and insulin sensitivity increased. NNMT inhibitors disclosed herein may therefore be useful in the treatment of metabolic disorders.

A compound or formulation of the invention may be used in the treatment of a metabolic disease, optionally wherein the metabolic disease is selected from metabolic syndrome, diabetes and obesity. The treatment may further comprise the administration of another active agent. In this context, the other active agent may itself be a prodrug (i.e. a compound transformed in vivo to the parent compound or other active compound), or the other active agent may be active in the form it is administered. Where the treatment comprises the administration of another active agent, the treatment may be by combined, sequential or separate administration of the present compound or formulation and the other active agent. For example, the treatment may comprise combined administration of the present compound and the other active agent. For example, the treatment may comprise administration of the present compound followed by administration of the other active agent.

Links to aberrant NNMT expression have also been found in neurodegenerative diseases, including Alzheimer's disease, Parkinson's disease, Huntington's diseases and schizophrenia (Schmeisser K, Parker J A. Nicotinamide-N-methyltransferase controls behavior, neurodegeneration and lifespan by regulating neuronal autophagy. PLOS Genet 2018; 14: e1007561; Lautrup S, et al. NAD+ in Brain Aging and Neurodegenerative Disorders. Cell Metab 2019; 30: 630-55; Parsons R B, et al. High expression of nicotinamide N-methyltransferase in patients with idiopathic Parkinson's disease. Neurosci Lett 2003; 342: 13-6; and Kocinaj A, Chaudhury T, Uddin M S, Junaid R R, Ramsden D B, Hondhamuni G, et al. High Expression of Nicotinamide N-Methyltransferase in Patients with Sporadic Alzheimer's Disease. Mol Neurobiol 2021). NNMT inhibitors disclosed herein may therefore be useful in the treatment of neurodegenerative diseases.

A compound or formulation of the invention may be used in the treatment of a neurodegenerative disease; optionally wherein the neurodegenerative disease is selected from Alzheimer's disease, Parkinson's disease, Huntington's diseases and schizophrenia. The treatment may further comprise the administration of another active agent. In this context, the other active agent may itself be a prodrug (i.e. a compound transformed in vivo to the parent compound or other active compound), or the other active agent may be active in the form it is administered. Where the treatment comprises the administration of another active agent, the treatment may be by combined, sequential or separate administration of the present compound or formulation and the other active agent. For example, the treatment may comprise combined administration of the present compound and the other active agent. For example, the treatment may comprise administration of the present compound followed by administration of the other active agent.

Aberrant NNMT expression has also been identified in functional disorders of the endothelium, such as thrombosis, high blood pressure, atherosclerosis, inflammation and pulmonary hypertension (Fedorowicz A, et al. Activation of the nicotinamide N-methyltransferase (NNMT)-1-methylnicotinamide (MNA) pathway in pulmonary hypertension. Respir Res 2016; 17: 108). NNMT inhibitors disclosed herein may therefore be useful in the treatment of functional disorders of the endothelium.

A compound or formulation of the invention may be used in the treatment of a functional disorder of the endothelium; optionally wherein the functional disorder of the endothelium is selected from thrombosis, high blood pressure, atherosclerosis, inflammation and pulmonary hypertension. The treatment may further comprise the administration of another active agent. In this context, the other active agent may itself be a prodrug (i.e. a compound transformed in vivo to the parent compound or other active compound), or the other active agent may be active in the form it is administered. Where the treatment comprises the administration of another active agent, the treatment may be by combined, sequential or separate administration of the present compound or formulation and the other active agent. For example, the treatment may comprise combined administration of the present compound and the other active agent. For example, the treatment may comprise administration of the present compound followed by administration of the other active agent.

Also provided is a method of treating a condition which is modulated by the inhibition of NNMT, comprising administering to the patient an effective amount of a compound of the invention or formulation of the invention. Optionally, wherein the condition is a condition selected from the group consisting of: cancer (such as lung cancer, bladder cancer, breast cancer, colorectal cancer, gastric cancer, hepatocellular cancer, ovarian cancer, pancreatic cancer, prostate cancer, oral cancer, glioma, lymphoma, and insulinoma), metabolic disorders, metabolic syndrome, diabetes, neurodegenerative diseases, Alzheimer's disease, Parkinson's disease, Huntington's diseases, schizophrenia, functional disorders of the endothelium, thrombosis, high blood pressure, atherosclerosis, inflammation and pulmonary hypertension. The method may further comprise the administration of another active agent. In this context, the other active agent may itself be a prodrug (i.e. a compound transformed in vivo to the parent compound or other active compound), or the other active agent may be active in the form it is administered. Where the treatment comprises the administration of another active agent, the treatment may be by combined, sequential or separate administration of the present compound or formulation and the other active agent. For example, the method may comprise combined administration of the present compound and the other active agent. For example, the method may comprise administration of the present compound followed by administration of the other active agent.

Methods are provided for inhibiting NNMT in vitro or in vivo, comprising administration to a cell of an effective amount of a compound of the invention or formulation of the invention.

### Formulations and Administration

Compounds of the invention may be administered orally, topically, intravenously, subcutaneously, buccally, rectally, dermally, nasally, tracheally, bronchially, by any other parenteral route, as an oral or nasal spray or via inhalation. The compounds may be administered in the form of pharmaceutical preparations comprising the compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Typically, therefore, the pharmaceutical compounds of the invention may be administered orally, topically, or parenterally (“parenterally” as used herein, refers to modes of administration which include intravenous, intramuscular, intraperitoneal, intrasternal, subcutaneous and intraarticular injection and infusion) to a host to obtain a protease-inhibitory effect. In the case of larger animals, such as humans, the compounds may be administered alone or as compositions in combination with pharmaceutically acceptable diluents, excipients or carriers.

Actual dosage levels of active ingredients in the pharmaceutical formulations and pharmaceutical compositions of this invention may be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

In the treatment, prevention, control, amelioration, or reduction of risk of conditions which require inhibition of NNMT activity, an appropriate dosage level may generally be about 0.01 to 500 mg per kg patient body weight per day which can be administered in single or multiple doses. The dosage level will be about 0.1 to about 250 mg/kg per day; more preferably about 0.5 to about 100 mg/kg per day. For oral administration, the compositions may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of the active ingredient for the symptomatic adjustment of the dosage to the patient to be treated. The compounds may be administered on a regimen of 1 to 4 times per day, e.g. once or twice per day. The dosage regimen may be adjusted to provide the optimal therapeutic response.

According to a further aspect of the invention there is thus provided a pharmaceutical formulation or composition including a compound of the invention, optionally in admixture with a pharmaceutically acceptable adjuvant, diluents or carrier.

Pharmaceutical formulations or compositions of this invention for parenteral injection may comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservative, wetting agents, emulsifying agents and dispersing agents. Inhibition of the action of microorganisms may be ensured by the inclusion of various antibacterial and antifungal agents, for example, paraben, chlorobutanol or phenol sorbic acid. It may also be desirable to include isotonic agents, such as sugars or sodium chloride, for example. Prolonged absorption of the injectable pharmaceutical form may be brought about by the inclusion of agents (for example, aluminium monostearate and gelatine) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms may be made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions which are compatible with body tissues. The injectable formulations can be sterilized, for example, by filtration through a bacterial retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier, such as sodium citrate or dicalcium phosphate and/or one or more: a) fillers or extenders, such as starches, lactose, sucrose, glucose, mannitol and silicic acid; b) binders, such as carboxymethylcellulose, alginates, gelatin, polyvinylpyrrolidone, sucrose and acacia; c) humectants, such as glycerol; d) disintegrating agents, such as agar-agar, calcium carbonate, potato or tapioca starch, alginic acid, certain silicates and sodium carbonate; e) solution retarding agents, such as paraffin; f) absorption accelerators, such as quaternary ammonium compounds; g) wetting agents, such as cetyl alcohol and glycerol monostearate; h) absorbents, such as kaolin and bentonite clay and i) lubricants, such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Oral formulations may contain a dissolution aid. Examples of dissolution aids include nonionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkyamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsufonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

The active compounds may also be in microencapsulated form, if appropriate, with one or more of the above-mentioned excipients.

The active compounds may be in finely divided form, for example it may be micronized.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavouring and perfuming agents. Suspensions, in addition to the active compounds, may contain suspending agents such as ethoxylated isostearyl alcohols, polyoxyethylene sorbitol and sorbitan esters, microcrystalline cellulose, aluminium metahydroxide, bentonite, agar-agar, and traganacanth and mixtures thereof.

Compositions for rectal or vaginal administration may be in the form of suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, creams, foams, gels, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Insofar as they do not interfere with the activity of the compounds, the formulations according to the present subject matter may contain other active agents. Exemplary other active agents for use in such formulations include, in particular, anticancer agents, such as anticancer agents that have NNMT related chemoresistance. Examples of such anticancer agents include adriamycin, paclitaxel and 5-fluoro-uracil.

The formulations according to the present subject matter may also contain inactive components. Suitable inactive components are well known in the art and are described in standard textbooks, such as Goodman and Gillman's: The Pharmacological Bases of Therapeutics, 8th Ed., Gilman et al, Eds. Pergamon Press (1990), and Remington's Pharmaceutical Sciences, 17th Ed., Mack Publishing Co., Easton, Pa. (1990), both of which are incorporated by reference herein in their entirety.

The formulations may be used in combination with an additional pharmaceutical dosage form to enhance their effectiveness in treating any of the disorders described herein. In this regard, the present formulations may be administered as part of a regimen additionally including any other pharmaceutical and/or pharmaceutical dosage form known in the art as effective for the treatment of any of these disorders.

### Additional Embodiments

The invention and disclosure also includes the subject matter of the following numbered clauses:
1. A compound of formula I,

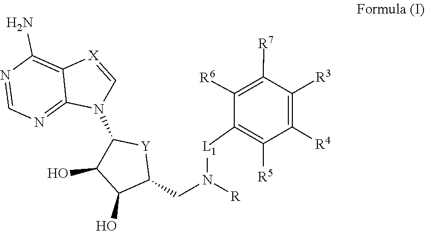

wherein:
   X is selected from N and CH;
   Y is selected from O and CH₂;
   L₁ is selected from the group consisting of: C₂-C₅ alkyl, C₃-C₅ alkenyl and C₃-C₅ alkynyl;
   R is selected from the group consisting of:

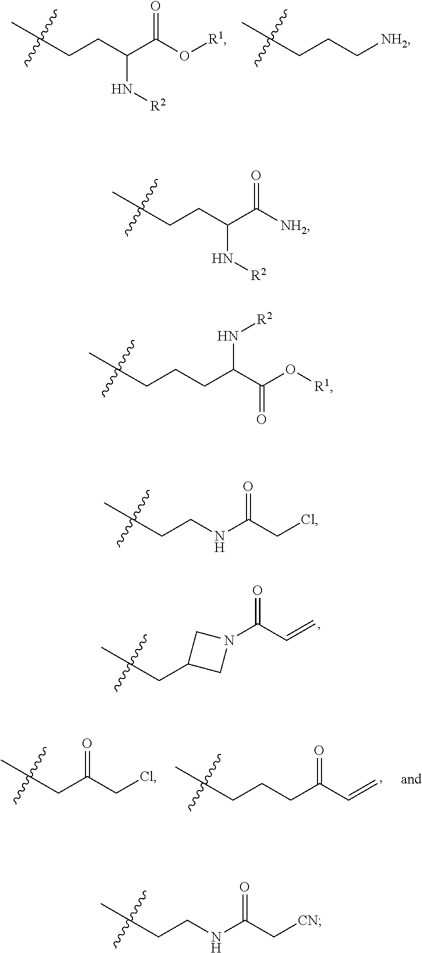

R¹ is selected from the group consisting of: H and a masking group;
R² is selected from the group consisting of: H and a masking group;
R³ is selected from the group consisting of: H and an electron withdrawing group;
when R³ is an electron withdrawing group, R⁴ is selected from H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g}; and when R³ is H, R⁴ is selected from C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g};
R⁵, R⁶ and R⁷ are each independently selected from H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g};
R^{a}, R^{b}, R^{c}, R^{d}, R^{e}, R^{f} and R^{g} are each independently at each occurrence selected from the group consisting of: H, halo, C₁-C₃ alkyl, C₂-C₅ alkenyl and C₁-C₃ haloalkyl;
or a pharmaceutically acceptable salt, stereoisomer, solvate or prodrug thereof.
2. The compound of clause 1, wherein L₁ is an unsubstituted C₃-C₅ alkenyl, optionally wherein L₁ is —CH₂CHCH—.
3. The compound of clause 1, wherein L₁ is selected from the group consisting of: C₁-alkyl, C₃-alkenyl and C₃-alkynyl.
4. The compound of any preceding clause, wherein R³ is an electron withdrawing group.
5. The compound of any preceding clause, wherein the compound is a compound of formula II,

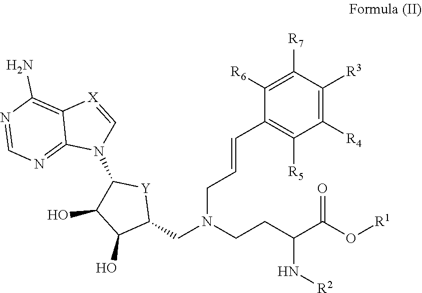

or a pharmaceutically acceptable salt, stereoisomer, solvate or prodrug thereof.
6. The compound of any preceding clause, wherein X is N.
7. The compound of any preceding clause, wherein Y is O.
8. The compound of any preceding clause, wherein R¹ is selected from the group consisting of: H and a masking group, wherein the masking group is substituted or unsubstituted C₁-C₆ alkyl, lipids, substituted or unsubstituted benzyl, substituted or unsubstituted aryl, and

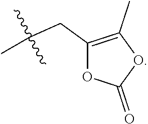 [SMILES: *Cc1oc(=O)oc1C]

9. The compound of any preceding clause, wherein R¹ is selected from the group consisting of: H and substituted or unsubstituted C₁-C₄ alkyl.
10. The compound of any preceding clause, wherein R¹ is selected from the group consisting of methyl, ethyl, propyl, isopropyl, benzyl and H.
11. The compound of any preceding clause, wherein R¹ is H.
12. The compound of any of clauses 1 to 10, wherein R¹ is selected from the group consisting of methyl, ethyl, propyl, isopropyl, benzyl.
13. The compound of any preceding clause, wherein R² is H.
14. The compound of any one of clauses 1 to 13, wherein R² is a masking group, optionally wherein the masking group is selected from the group consisting of:

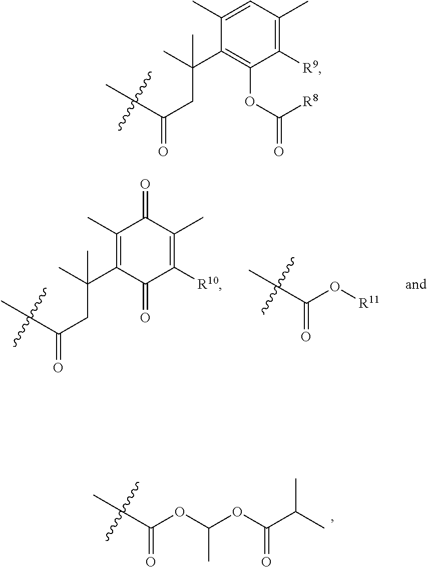

wherein
   R³ is C₁-C₆ alkyl or aryl; R⁹ is H or methyl; R¹⁰ is H or methyl; and R¹¹ is C₁-C₆ alkyl or aryl.
15. The compound of any one of clauses 1 to 10, wherein R¹ is H and R² is H.
16. The compound of any preceding clause, wherein R³ is selected from the group consisting of: halo, CN, NO₂, CF₃ and SO₂F.
17. The compound of any preceding clause, wherein R³ is CN.
18. The compound of any preceding clause, wherein R⁴, R⁵, R⁶ and R⁷ are each independently selected from H, haloalkyl, halo, NO₂, CN and C(O)NR^{c}R^{d}.
19. The compound of any preceding clause, wherein each of R⁴ and R⁷ are independently selected from H, halo, NO₂, CN and C(O)NR^{c}R^{d}; R⁵ is H; and R⁶ is H.
20. The compound of any of clauses 1 to 18, wherein both of R⁴ and R⁷ are F, and both of R⁵ and R⁶ are H.
21. The compound of any of clauses 1 to 18, wherein each of R⁴, R⁵, R⁶ and R⁷ are halo (optionally F), or H.
22. The compound of any of clauses 1 to 18, wherein each of R⁴, R⁵, R⁶ and R⁷ are H.
23. The compound of any of clauses 1 to 18, wherein each of R⁴, R⁵, R⁶ and R⁷ are F.
24. The compound of clause 1, wherein the compound is a compound selected from:

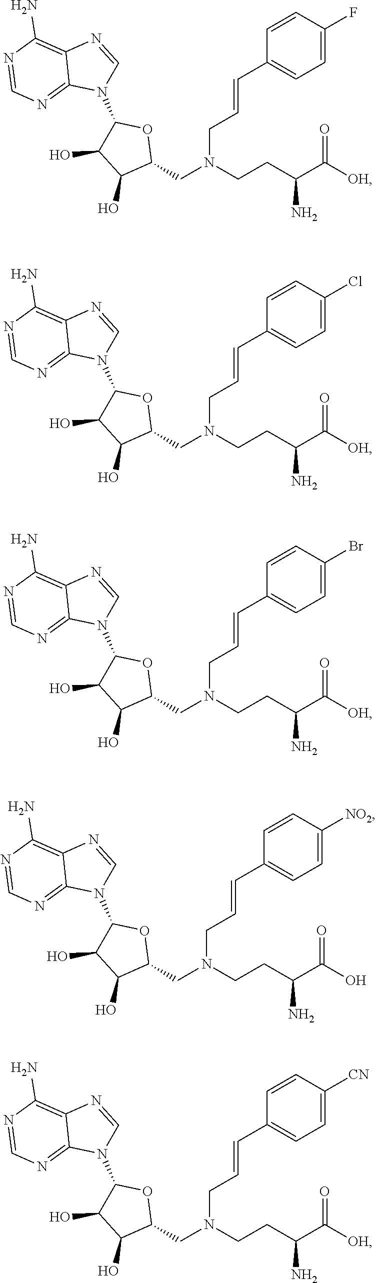 [SMILES: Nc1ncnc2c1ncn2[C@@H]1O[C@H](CN(C/C=C/c2ccc(F)cc2)CC[C@H](N)C(=O)O)[C@@H](O)[C@H]1O ; Nc1ncnc2c1ncn2[C@@H]1O[C@H](CN(C/C=C/c2ccc(Cl)cc2)CC[C@H](N)C(=O)O)[C@@H](O)[C@H]1O ; Nc1ncnc2c1ncn2[C@@H]1O[C@H](CN(C/C=C/c2ccc(Br)cc2)CC[C@H](N)C(=O)O)[C@@H](O)[C@H]1O ; Nc1ncnc2c1ncn2[C@@H]1O[C@H](CN(C/C=C/c2ccc([N+](=O)[O-])cc2)CC[C@H](N)C(=O)O)[C@@H](O)[C@H]1O ; N#Cc1ccc(/C=C/CN(CC[C@H](N)C(=O)O)C[C@H]2O[C@@H](n3cnc4c(N)ncnc43)[C@H](O)[C@@H]2O)cc1]

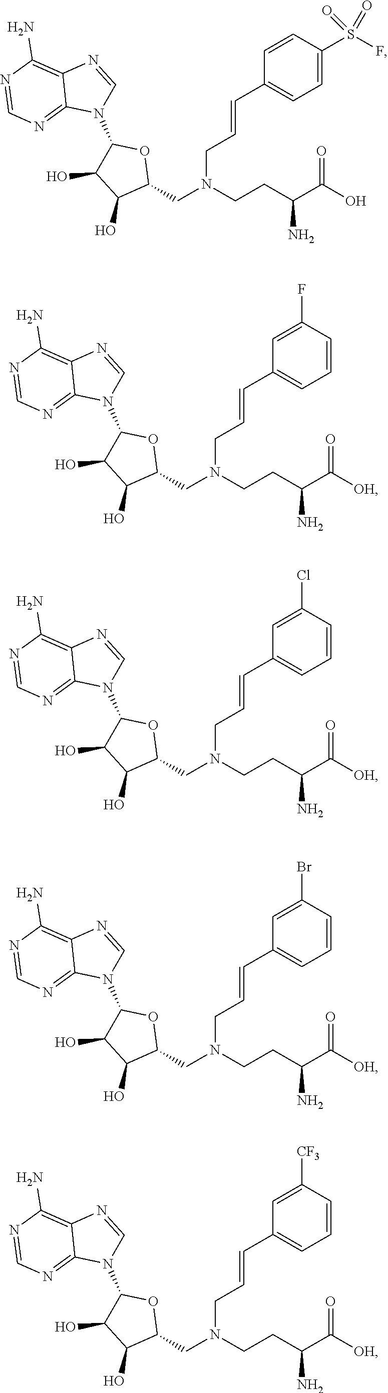 [SMILES: Nc1ncnc2c1ncn2[C@@H]1O[C@H](CN(C/C=C/c2ccc(S(=O)(=O)F)cc2)CC[C@H](N)C(=O)O)[C@@H](O)[C@H]1O ; Nc1ncnc2c1ncn2[C@@H]1O[C@H](CN(C/C=C/c2cccc(F)c2)CC[C@H](N)C(=O)O)[C@@H](O)[C@H]1O ; Nc1ncnc2c1ncn2[C@@H]1O[C@H](CN(C/C=C/c2cccc(Cl)c2)CC[C@H](N)C(=O)O)[C@@H](O)[C@H]1O ; Nc1ncnc2c1ncn2[C@@H]1O[C@H](CN(C/C=C/c2cccc(Br)c2)CC[C@H](N)C(=O)O)[C@@H](O)[C@H]1O ; Nc1ncnc2c1ncn2[C@@H]1O[C@H](CN(C/C=C/c2cccc(C(F)(F)F)c2)CC[C@H](N)C(=O)O)[C@@H](O)[C@H]1O]

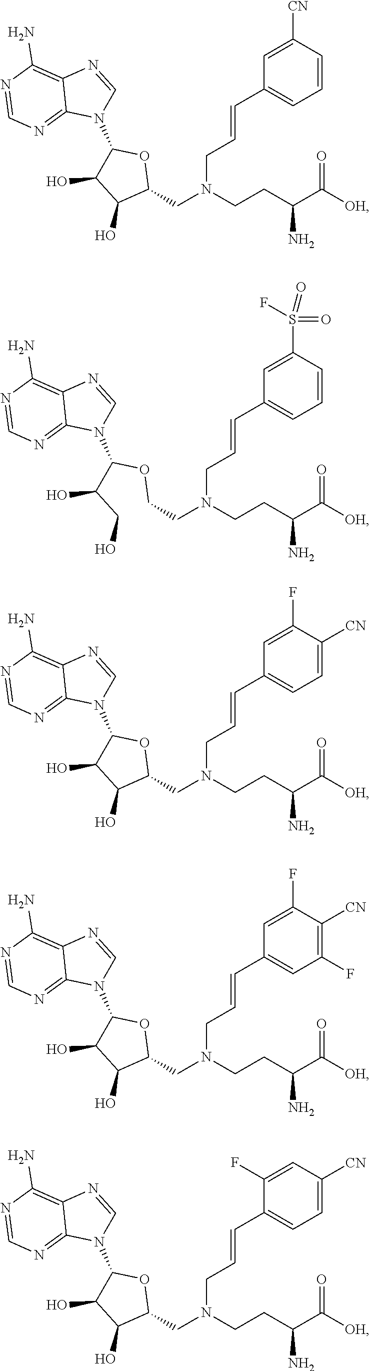

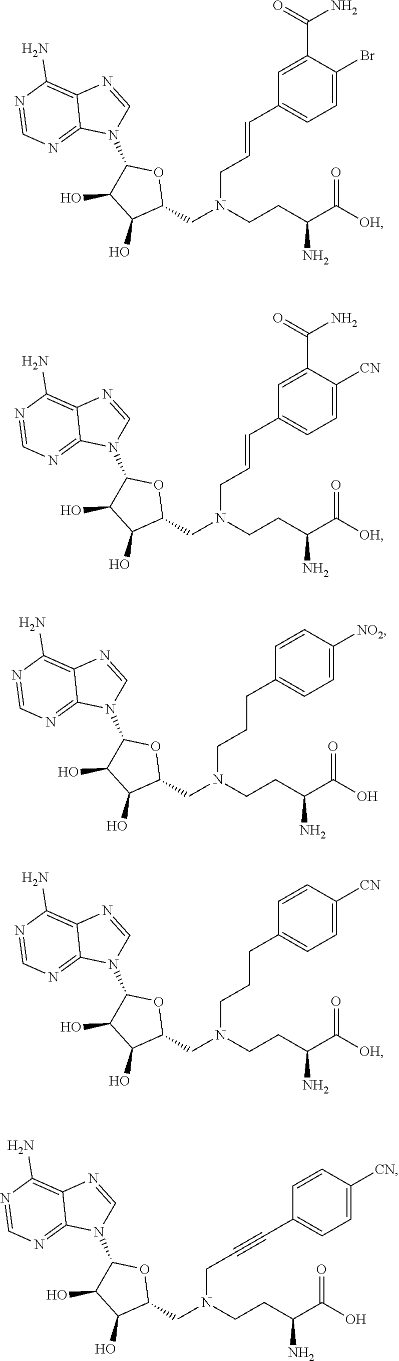 [SMILES: NC(=O)c1cc(/C=C/CN(CC[C@H](N)C(=O)O)C[C@H]2O[C@@H](n3cnc4c(N)ncnc43)[C@H](O)[C@@H]2O)ccc1Br ; N#Cc1ccc(/C=C/CN(CC[C@H](N)C(=O)O)C[C@H]2O[C@@H](n3cnc4c(N)ncnc43)[C@H](O)[C@@H]2O)cc1C(N)=O ; Nc1ncnc2c1ncn2[C@@H]1O[C@H](CN(CCCc2ccc([N+](=O)[O-])cc2)CC[C@H](N)C(=O)O)[C@@H](O)[C@H]1O ; N#Cc1ccc(CCCN(CC[C@H](N)C(=O)O)C[C@H]2O[C@@H](n3cnc4c(N)ncnc43)[C@H](O)[C@@H]2O)cc1 ; N#Cc1ccc(C#CCN(CC[C@H](N)C(=O)O)C[C@H]2O[C@@H](n3cnc4c(N)ncnc43)[C@H](O)[C@@H]2O)cc1]

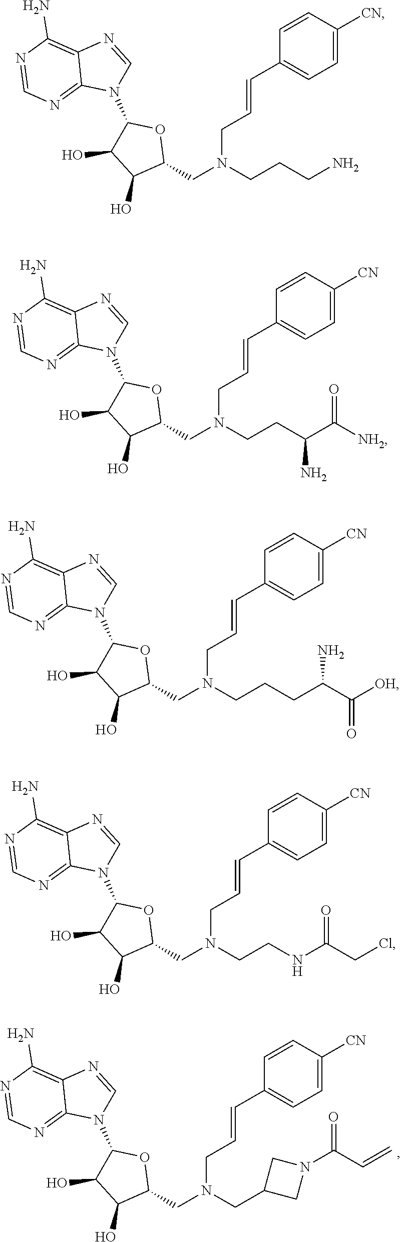 [SMILES: N#Cc1ccc(/C=C/CN(CCCN)C[C@H]2O[C@@H](n3cnc4c(N)ncnc43)[C@H](O)[C@@H]2O)cc1 ; N#Cc1ccc(/C=C/CN(CC[C@H](N)C(N)=O)C[C@H]2O[C@@H](n3cnc4c(N)ncnc43)[C@H](O)[C@@H]2O)cc1 ; N#Cc1ccc(/C=C/CN(CCC[C@H](N)C(=O)O)C[C@H]2O[C@@H](n3cnc4c(N)ncnc43)[C@H](O)[C@@H]2O)cc1 ; N#Cc1ccc(/C=C/CN(CCNC(=O)CCl)C[C@H]2O[C@@H](n3cnc4c(N)ncnc43)[C@H](O)[C@@H]2O)cc1 ; C=CC(=O)N1CC(CN(C/C=C/c2ccc(C#N)cc2)C[C@H]2O[C@@H](n3cnc4c(N)ncnc43)[C@H](O)[C@@H]2O)C1]

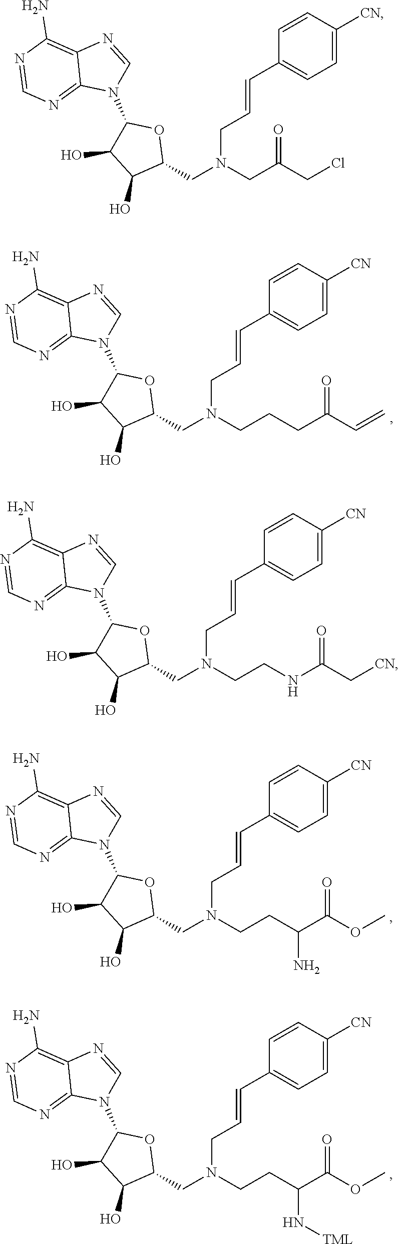

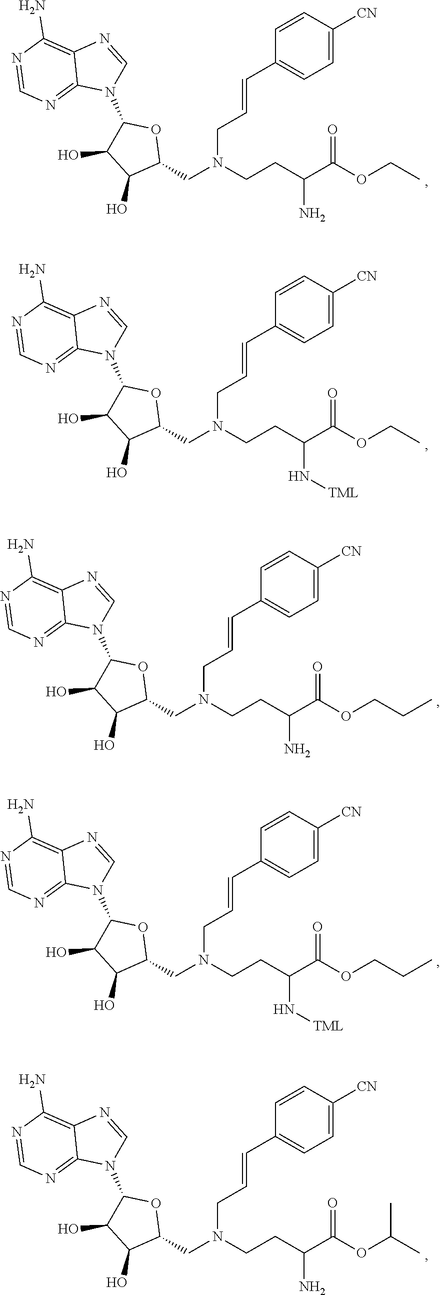

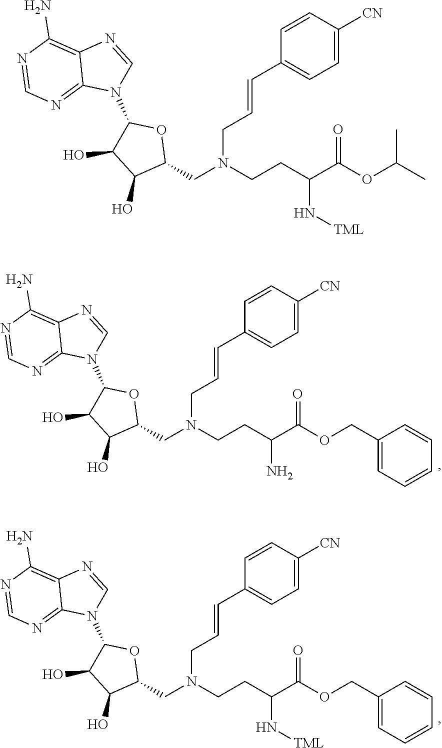

where TML is selected from

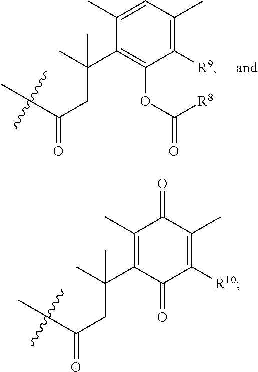

R⁸, R⁹, and R¹⁰ are as defined in claim 14;
or a pharmaceutically acceptable salt, stereoisomer, or solvate thereof.
25. A pharmaceutical formulation comprising a compound of any of clauses 1 to 24 and optionally a pharmaceutically acceptable carrier.
26. The pharmaceutical formulation of clause 25, further comprising an additional pharmaceutically active agent, optionally wherein the additional pharmaceutically active agent is an anticancer agent.
27. A compound of any of clauses 1 to 24, or a formulation of clause 25 or clause 26, for use as a medicament.
28. A compound of any of clauses 1 to 24, or a formulation of clause 25 or clause 26, for use in the treatment of cancer; optionally wherein the cancer is selected from lung cancer, bladder cancer, renal cancer, oral cancer, skin cancer, breast cancer, colorectal cancer, gastric cancer, hepatocellular cancer, ovarian cancer, pancreatic cancer, prostate cancer and glioblastoma.
29. A compound of any of clauses 1 to 24, or a formulation of clause 25 or clause 26, for use in the treatment of metabolic disease; optionally wherein the metabolic disease is selected from metabolic syndrome, diabetes and obesity.
30. A compound of any of clauses 1 to 24, or a formulation of clause 25 or clause 26, for use in the treatment of neurodegenerative disease; optionally wherein the neurodegenerative disease is selected from Alzheimer's disease, Parkinson's disease, Huntington's diseases and schizophrenia.
31. A compound of any of clauses 1 to 24, or a formulation of clause 25 or clause 26, for use in the treatment of a functional disorder of the endothelium; optionally wherein the functional disorder of the endothelium is selected from thrombosis, high blood pressure, atherosclerosis, inflammation and pulmonary hypertension.
32. A compound of any of clauses 1 to 24, or a formulation of clause 25 or clause 26, for use in the treatment of a condition treated by the inhibition of Nicotinamide N-methyltransferase (NNMT), optionally wherein the condition is modulated by NAD dependent signalling.
33. A compound or pharmaceutical composition for use of clause 32, wherein the condition is selected from the group consisting of: cancer (such as lung cancer, bladder cancer, renal cancer, oral cancer, skin cancer, breast cancer, colorectal cancer, gastric cancer, hepatocellular cancer, ovarian cancer, pancreatic cancer, prostate cancer and glioblastoma), metabolic disorders, diabetes, neurodegenerative diseases, Alzheimer's disease, Parkinson's disease, Huntington's diseases, schizophrenia, functional disorders of the endothelium, thrombosis, high blood pressure, atherosclerosis, inflammation and pulmonary hypertension.
34. A method of treatment of a condition which is modulated by the inhibition of NNMT, wherein the method comprises administering a therapeutic amount of a compound or composition of any of clauses 1 to 24, to a patient in need thereof.
35. The method of clause 34, wherein the condition is selected from the group consisting of: cancer (such as lung cancer, bladder cancer, renal cancer, oral cancer, skin cancer, breast cancer, colorectal cancer, gastric cancer, hepatocellular cancer, ovarian cancer, pancreatic cancer, prostate cancer and glioblastoma), metabolic disorders, metabolic syndrome, diabetes, neurodegenerative diseases, Alzheimer's disease, Parkinson's disease, Huntington's diseases, schizophrenia, functional disorders of the endothelium, thrombosis, high blood pressure, atherosclerosis, inflammation and pulmonary hypertension.
36. A compound of any of clauses 1 to 24, or a formulation of clause 25 or clause 26, for the inhibition of NNMT in vitro or in vivo.

### Assays

### Biochemical and Biological Assays:

### 1) Description of NNMT Assay (IC50 Results Provided in the Tables Below)

The bisubstrate analogues were tested for their NNMT inhibitory activity using a method recently developed in our group (van Haren M J, Sastre Toraño J, Sartini D, Emanuelli M, Parsons R B, Martin N I. A Rapid and Efficient Assay for the Characterization of Substrates and Inhibitors of Nicotinamide N-Methyltransferase. Biochemistry 2016; 55: 5307-15). This assay employs hydrophilic liquid interaction chromatography (HILIC) coupled with tandem mass spectrometry (MS/MS) to rapidly and efficiently assess NNMT inhibition by analysis of the formation of 1-methyl-nicotinamide (MNA). The use of an isotope-labeled deuteromethyl-nicotinamide (d3-MNA) internal standard allows for the quantification of MNA.

The enzymatic activity assays were performed using NNMT at a final concentration of 100 nM diluted in assay buffer (50 mM Tris buffer (pH 8.4) and 1 mM dithiothreitol). The compounds were dissolved in DMSO and diluted with water to concentrations ranging from 0.1 nM to 500 μM (DMSO was kept constant at 1.25% final concentration). The compounds were incubated with the enzyme for 10 min at room temperature before initiating the reaction with a mixture of nicotinamide (NA) and S-adenosyl-L-methionine (SAM) at their KM values of 200 and 8.5 μM, respectively. The formation of MNA was measured after 30 min at room temperature. The reaction was quenched by addition of 30 μL of the sample to 70 μL of acetonitrile containing 50 nM d3-MNA as internal standard.

The samples were analyzed for MNA through isocratic elution of 5 μL injections on a Waters Acquity BEH Amide HILIC column (3.0×100 mm, 1.7 μm particle size, Waters, Milford), using water containing 300 mM formic acid and 550 mM NH4OH (pH 9.2) at 40% v/v and acetonitrile at 60% v/v, with a runtime of 2 min. Calibration samples were prepared using 70 μL of internal standard d3-MNA at 50 nM in acetonitrile and 30 μL of an aqueous solution of reference standard MNA with concentrations ranging from 1 to 1024 nM. Ratios of the sums of the MNA and d3-MNA transitions were calculated and plotted versus concentration.

The NNMT inhibition of all compounds was initially screened at a fixed concentration of 25 μM for all of the compounds. In cases where at least 50% inhibition was detected at this concentration, full inhibition curves were measured in triplicate to determine the corresponding half-maximal inhibitory concentration (IC50) values.

The data was fitted using non-linear regression analysis of the Sigmoidal dose-response curve generated using normalized data and a variable slope. The IC50 value was determined by the concentration resulting in a half-maximal percent activity. Values are reported along with standard errors of the mean (S.E.M., calculated using the symmetrical Cl function in Graphpad Prism 8) indicating the precision of the mean values obtained.

### 2) Description of MTT Cell-Based Assays and Results

Cell Culture and Treatment with Compounds

UROTsa immortalized human urothelium cell line, HSC-2 human oral cancer cell line, T24 human bladder cancer cell line and A549 human lung cancer line were purchased from the American Type Culture Collection (ATCC, Rockville, MD, USA), and cultured in DMEM/F12 medium, supplemented with 10% fetal bovine serum and 50 μg/ml gentamicin, at 37° C. in a humidified 5% CO2 incubator. For each compound tested, powder was dissolved in DMSO at 100 mM concentration. This stock solution was then diluted in culture medium to final concentration values ranging between 1 μM and 100 μM. For each sample, DMSO was kept constant at 0.1% final concentration. The day before starting treatment, cells were seeded in 96-well plates, at a density of 2000 cells/well. Cells were allowed to attach overnight and then incubated with compounds at different final concentrations, or with DMSO only, for 24, 48 and 72 hours. All experiments were performed in triplicate.

### MTT Assay

Cell proliferation was determined using a colorimetric assay with 3-(4,5-dimethylthiazol-2-yl)-2,5-diphenyl tetrazolium bromide (MTT). The MTT assay measures the conversion of MTT to insoluble formazan by dehydrogenase enzymes of the intact mitochondria of living cells. Cell proliferation was evaluated by measuring the conversion of the tetrazolium salt MTT to formazan crystals upon treatment with compounds or DMSO only for 24, 48 and 72 hours. Briefly, cells were incubated for 2 hours at 37° C. with 100 μl fresh culture medium containing 5 μl of MTT reagent (5 mg/ml in PBS). The medium was removed and 200 μl isopropanol were added. The amount of formazan crystals formed correlated directly with the number of viable cells. The reaction product was quantified by measuring the absorbance at 540 nm using an ELISA plate reader. Experiments were repeated three times. Results were expressed as percentage of the control (control equals 100% and corresponds to the absorbance value of each sample at time zero) and presented as mean values±standard deviation of three independent experiments performed in triplicate.

### 3) Statistical Analysis

Data were analysed using GraphPad Prism software for Windows (GraphPad Software, San Diego, CA). Significant differences between groups were determined using the one-way analysis of variance (ANOVA). A p value <0.05 was considered as statistically significant.

### EXAMPLES

For each compound, the maximum concentration tested is 25 μM. In case at least 50% inhibition was measured, full IC50 curves were generated to obtain the IC50 values and their error. If not, the IC50 values are stated as >25 μM. The inhibition data and the errors are presented in both micromolar (μM) and nanomolar (nM) because of the wide range of activities found for the different compounds. For all compounds the results of high-resolution (HRMS) or low resolution (LRMS) mass spectrometry is given.

### Example 1

Compounds with Alkene Linkage and Variation of Aromatic Group

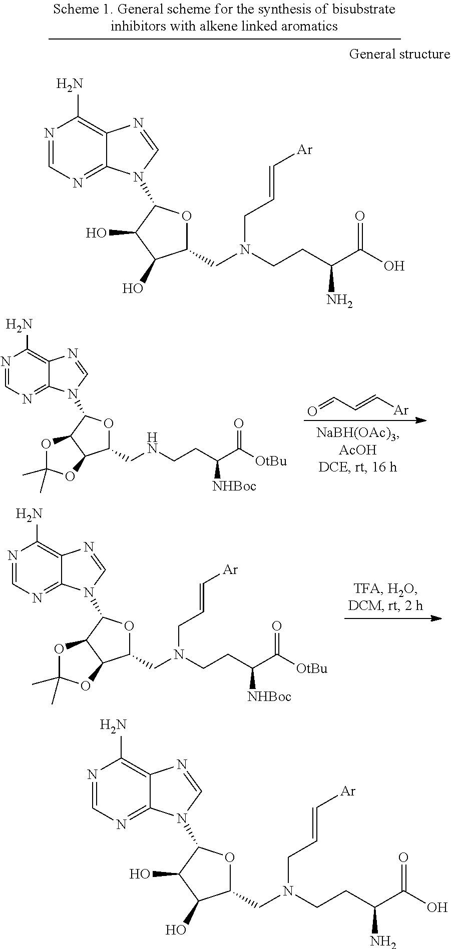

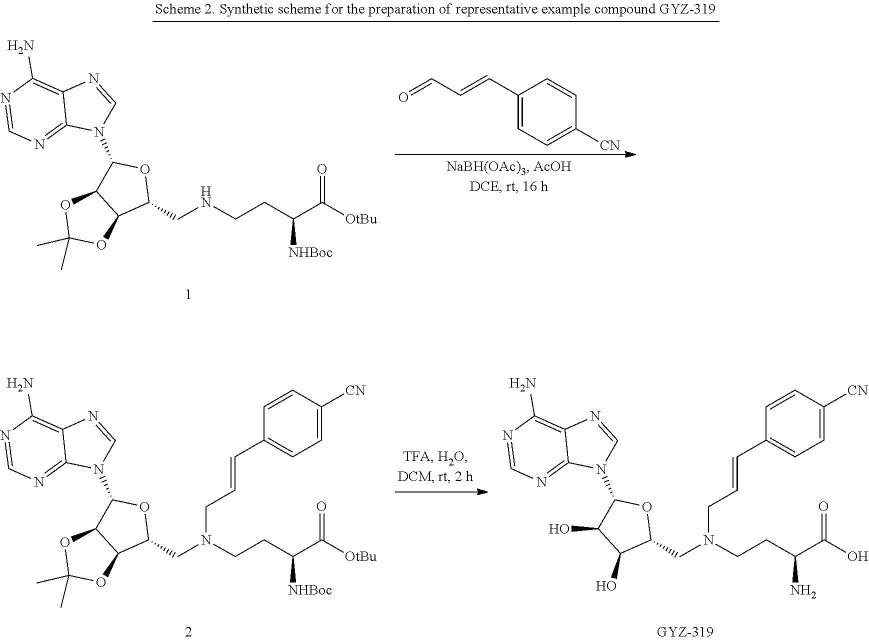 [SMILES: CC(C)(C)OC(=O)N[C@@H](CCNC[C@H]1O[C@@H](n2cnc3c(N)ncnc32)[C@@H]2OC(C)(C)O[C@@H]21)C(=O)OC(C)(C)C ; N#Cc1ccc(/C=C/C=O)cc1 ; CC(C)(C)OC(=O)N[C@@H](CCN(C/C=C/c1ccc(C#N)cc1)C[C@H]1O[C@@H](n2cnc3c(N)ncnc32)[C@@H]2OC(C)(C)O[C@@H]21)C(=O)OC(C)(C)C ; N#Cc1ccc(/C=C/CN(CC[C@H](N)C(=O)O)C[C@H]2O[C@@H](n3cnc4c(N)ncnc43)[C@H](O)[C@@H]2O)cc1]

### Experimental Procedure for the Preparation of Representative Example Compound GYZ-319

Compound 1 (112 mg, 0.2 mmol), (E)-4-(3-oxoprop-1-en-1-yl)benzonitrile (58 mg, 0.24 mmol), NaBH(OAc)3 (11 mg, 0.3 mmol) and AcOH (one drop) were added to 10 mL DCE in a 50 mL round bottom flask (RBF), the mixture stirred at room temperature under N2 atmosphere overnight. The reaction was quenched by adding 1 N NaOH (10 mL), and the product was extracted with CH2Cl2. The combined organic layers were washed with brine and dried over Na2SO4. The solvent was evaporated, and the crude product was purified by column chromatography (5% MeOH in EtOAc) to give intermediate 2 as a white powder (94 mg, 67% yield).

To a solution of the protected intermediate 2 (50 mg, 0.071 mmol) in 1 mL of CH2Cl2 was added 9 mL TFA and 1 mL H2O, the mixture was stirred for 2 hours at room temperature. The mixture was concentrated, and the crude product was purified by preparative HPLC affording final compound GYZ-319 as a white powder (33 mg, 74% yield). 1H NMR (400 MHz, D2O) δ 8.38 (s, 1H), 8.14 (s, 1H), 7.59 (d, J=8.0 Hz, 2H), 7.16 (d, J=8.1 Hz, 2H), 6.47 (m, 1H), 6.23-6.13 (m, 2H), 4.74 (dd, J=7.0, 5.5 Hz, 1H), 4.60-4.55 (m, 1H), 4.47 (s, 1H), 4.02 (dd, J=8.6, 4.5 Hz, 4H), 3.61 (m, 3H), 2.47 (m, 1H), 2.32 (m, 1H). 13C NMR (101 MHz, D2O) δ 171.8, 149.3, 147.2, 144.1, 143.6, 138.1, 132.63, 126.6, 119.2, 114.9, 110.8, 91.4, 73.6, 71.9, 51.6, 20.5. HRMS (ESI): calculated for C24H28N8O5 [M+H]+ 509.2261, found 509.2266.

TABLE 1
IC₅₀ data and HRMS results for bisubstrate inhibitors with alkene linked
aromatics

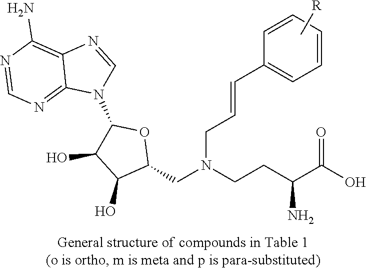

| | | | | Standard | | | |
|---|---|---|---|---|---|---|---|
| | | IC₅₀ values | | error | | HRMS | |
| Code | R | (μM) | (nM) | (μM) | (nM) | calculated | found |
| GYZ- | o-F | 8.978 | 8978.00 | 0.56 | 563.72 | [M + H]⁺ | 502.2225 |
| 311 | | | | | | 502.2214 | |
| GYZ- | m-F | 3.780 | 3780.07 | 0.48 | 483.95 | [M + H]⁺ | 502.2218 |
| 331 | | | | | | 502.2214 | |
| GYZ- | p-F | 0.187 | 187.40 | 0.01 | 9.68 | [M + H]⁺ | 502.2216 |
| 287 | | | | | | 502.2214 | |
| GYZ- | o-Cl | 1.343 | 1343.00 | 0.12 | 122.08 | [M + H]⁺ | 518.1922 |
| 361 | | | | | | 518.1919 | |
| GYZ- | m-Cl | 0.643 | 642.60 | 0.05 | 51.07 | [M + H]⁺ | 518.1928 |
| 357 | | | | | | 518.1919 | |
| GYZ- | p-Cl | 0.240 | 240.40 | 0.02 | 16.86 | [M + H]⁺ | 518.1925 |
| 288 | | | | | | 518.1919 | |
| GYZ- | o-Br | 1.453 | 1453.00 | 0.12 | 119.02 | [M + H]⁺ | 562.1427 |
| 344 | | | | | | 562.1414 | |
| GYZ- | m-Br | 0.377 | 377.00 | 0.02 | 19.59 | [M + H]⁺ | 562.1425 |
| 353 | | | | | | 562.1414 | |
| GYZ- | p-Br | 0.061 | 61.34 | 0.00 | 3.92 | [M + H]⁺ | 562.1421 |
| 289 | | | | | | 562.1414 | |
| GYZ- | o-NO₂ | >25 | >25000 | n/a | n/a | [M + H]⁺ | 529.2166 |
| 290 | | | | | | 529.2159 | |
| GYZ- | m-NO₂ | >25 | >25000 | n/a | n/a | [M + H]⁺ | 529.2178 |
| 348 | | | | | | 529.2159 | |
| GYZ- | p-NO₂ | 0.010 | 9.76 | 0.00 | 0.66 | [M + H]⁺ | 529.2271 |
| 316 | | | | | | 529.2259 | |
| GYZ- | o-CF₃ | 2.854 | 2854.00 | 0.17 | 171.24 | [M + H]⁺ | 552.2191 |
| 326 | | | | | | 552.2182 | |
| GYZ- | m-CF₃ | 0.164 | 164.30 | 0.01 | 7.70 | [M + H]⁺ | 529.2187 |
| 349 | | | | | | 552.2182 | |
| GYZ- | p-CF₃ | 1.386 | 1386.00 | 0.05 | 47.36 | [M + H]⁺ | 552.2184 |
| 350 | | | | | | 552.2182 | |
| GYZ- | o-CN | >25 | >25000 | n/a | n/a | [M + H]⁺ | 509.2271 |
| 347 | | | | | | 509.2261 | |
| GYZ- | m-CN | 0.859 | 859.10 | 0.04 | 43.42 | [M + H]⁺ | 509.2264 |
| 355 | | | | | | 509.2261 | |
| GYZ- | p-CN | 0.004 | 3.69 | 0.00 | 0.17 | [M + H]⁺ | 509.2266 |
| 319 | | | | | | 509.2261 | |
| GYZ- | o,o-bisCl | >25 | >25000 | n/a | n/a | [M + H]⁺ | 552.1536 |
| 336 | | | | | | 552.1529 | |
| GYZ- | o-CH₃ | 1.158 | 1158.43 | 0.05 | 54.86 | [M + H]⁺ | 498.2572 |
| 438 | | | | | | 498.2465 | |
| GYZ- | m-CH₃ | 6.439 | 6438.73 | 0.85 | 851.68 | [M + H]⁺ | 498.2574 |
| 432 | | | | | | 498.2465 | |
| GYZ- | p-CH₃ | 4.605 | 4604.69 | 0.37 | 371.81 | [M + H]⁺ | 498.2570 |
| 434 | | | | | | 498.2465 | |
| GYZ- | o-OCH₃ | 14.928 | 14927.94 | 0.89 | 890.78 | [M + H]⁺ | 514.2422 |
| 445 | | | | | | 514.2414 | |
| GYZ- | m-OCH₃ | 4.084 | 4084.13 | 0.20 | 195.96 | [M + H]⁺ | 514.2419 |
| 431 | | | | | | 514.2414 | |
| GYZ- | p-OCH₃ | 20.450 | 20450.00 | 1.66 | 1660.48 | [M + H]⁺ | 514.2425 |
| 433 | | | | | | 514.2414 | |
| GYZ- | H | 13.630 | 13630.00 | 0.76 | 759.20 | [M + H]⁺ | 484.2311 |
| 435 | | | | | | 484.2308 | |
| GYZ- | p-amide | 10.770 | 10770.00 | 1.09 | 1091.46 | [M + H]⁺ | 527.2373 |
| 566 | | | | | | 527.2367 | |
| GYZ- | m-amide | 0.013 | 12.76 | 0.00 | 0.78 | [M + H]⁺ | 527.2378 |
| 567 | | | | | | 527.2367 | |
| GYZ- | m-B(OH)₂ | 1.658 | 1658.27 | 0.13 | 134.38 | [M + H]⁺ | 528.2385 |
| 746 | | | | | | 528.2378 | |
| GYZ- | p-B(OH)₂ | >25 | >25000 | n/a | n/a | [M + H]⁺ | 528.2384 |
| 732 | | | | | | 528.2378 | |
| GYZ- | p-alkyne | >25 | >25000 | n/a | n/a | [M + H]⁺ | 508.2315 |
| 750 | | | | | | 508.2308 | |
| GYZ- | p-COOH | >25 | >25000 | n/a | n/a | [M + H]⁺ | 528 |
| 564 | | | | | | 528.2207 | (LRMS) |
| GYZ- | m-SO₂F | 0.089 | 88.58 | 0.01 | 8.90 | [M + H]⁺ | 566.1835 |
| 764 | | | | | | 566.1833 | |
| GYZ- | p-SO₂F | 0.068 | 67.93 | 0.00 | 3.24 | [M + H]⁺ | 566.1837 |
| 736 | | | | | | 566.1833 | |
| GYZ- | m-F, p-CN | <0.005 | <5 | 0.00 | 0.22 | [M + H]⁺ | 527.2174 |
| 658 | | | | | | 527.2167 | |
| GYZ- | m-F, m-F, p- | 0.002 | 1.99 | 0.00 | 0.31 | [M + H]⁺ | 545.2081 |
| 670 | CN | | | | | 545.2072 | |
| GYZ- | o-F, p-CN | 0.005 | 5.33 | 0.00 | 0.26 | [M + H]⁺ | 527.2168 |
| 674 | | | | | | 527.2167 | |
| GYZ- | p-Br, m- | 0.007 | 6.80 | 0.00 | 0.29 | [M + H]⁺ | 605.1477 |
| 739 | amide | | | | | 605.1472 | |
| GYZ- | p-CN, m- | 0.029 | 28.73 | 0.00 | 2.36 | [M + H]⁺ | 552.2324 |
| 755 | amide | | | | | 552.2319 | |
| MH609 | pentafluoro | 0.400 | 399.52 | 0.04 | 43.06 | [M + H]⁺ | 574.1834 |
| | | | | | | 574.1832 | |
| MH610 | tetrafluoro | 0.007 | 7.14 | 0.00 | 0.78 | [M + H]⁺ | 581.1882 |
| | para-CN | | | | | 581.1879 | |
| MH611 | m,m,p- | 0.363 | 363.49 | 0.04 | 42.68 | [M + H]⁺ | 538.2021 |
| | trifluoro | | | | | 538.2020 | |
| MH612 | o,o,m,m | 1.814 | 1813.77 | 0.20 | 204.78 | [M + H]⁺ | 556.1927 |
| | tetrafluoro | | | | | 556.1926 | |

### Example 2

### Exemplary Prodrug Compounds

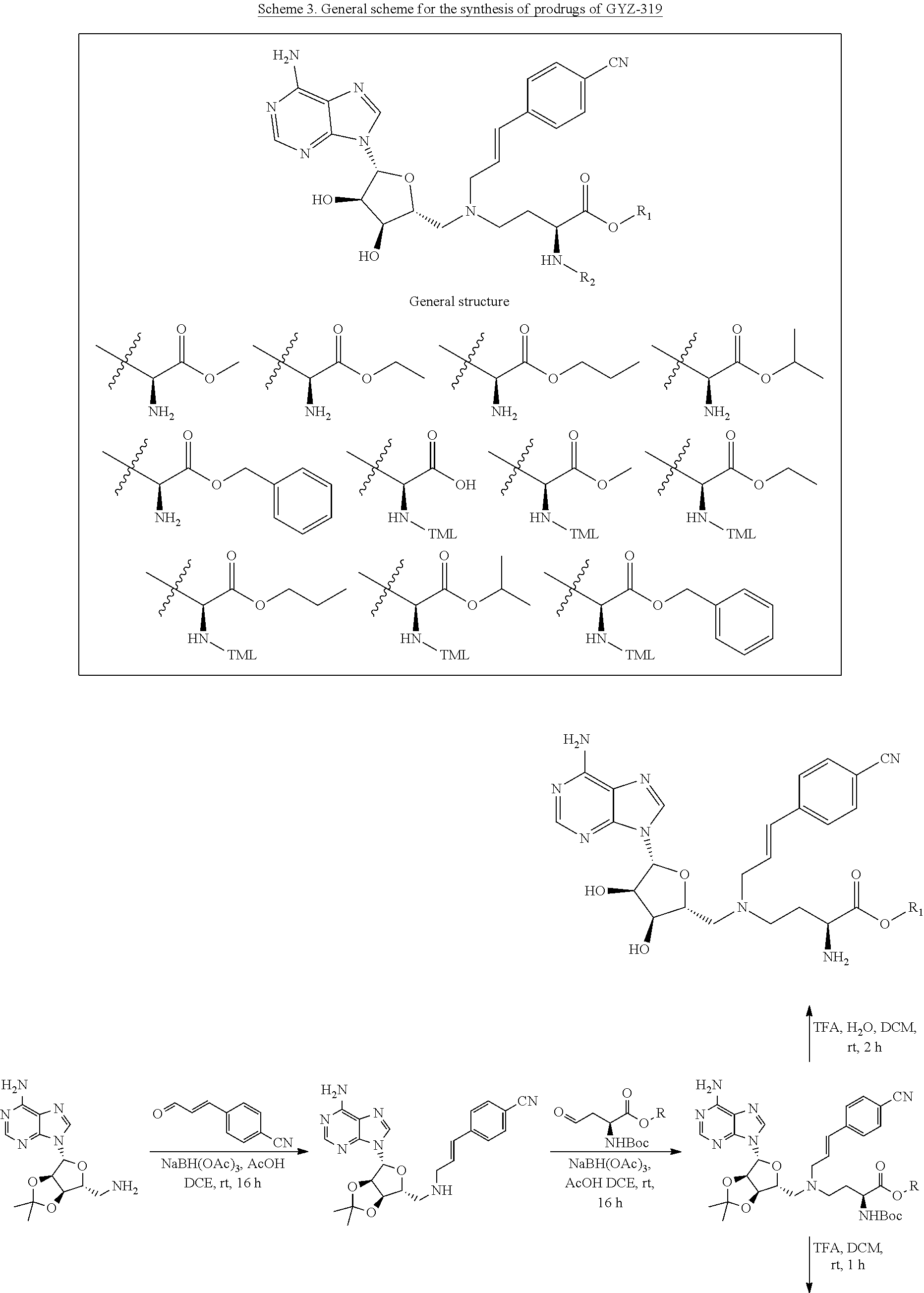

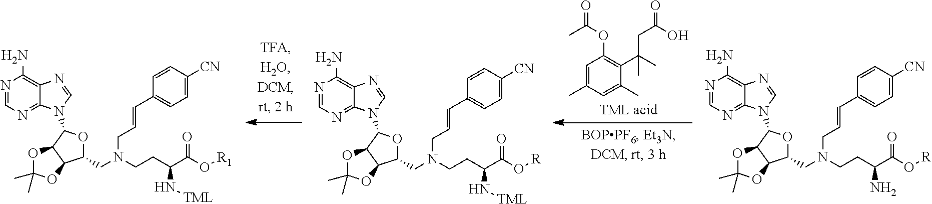

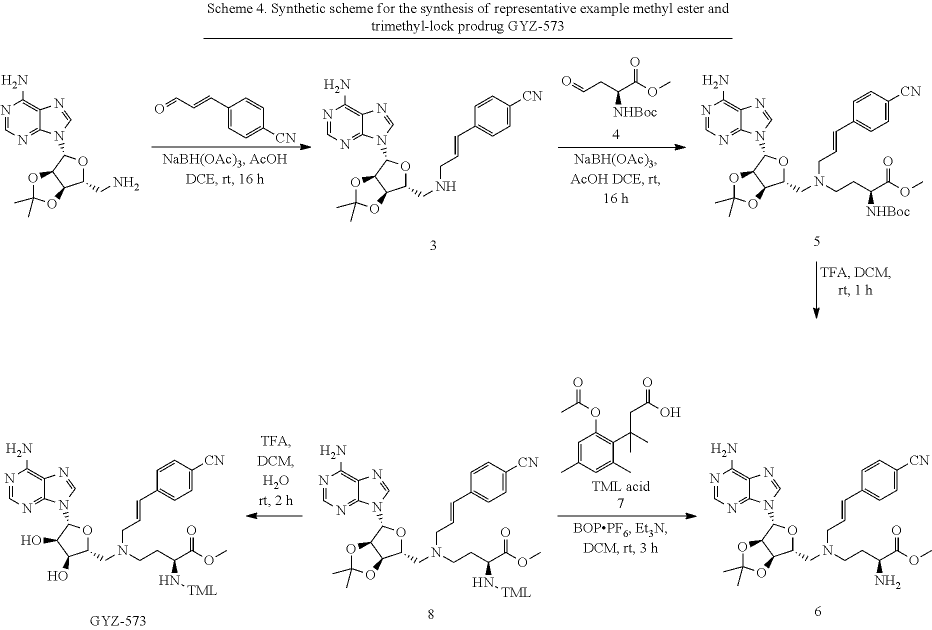

### Experimental Procedure for the Preparation of Representative Example Compound GYZ-573

9-((3aR,4R,6R,6aR)-6-(aminomethyl)-2,2-dimethyltetrahydrofuro[3,4-d][1,3]dioxol-4-yl)-9H-purin-6-amine (67 mg, 0.22 mmol), (E)-4-(3-oxoprop-1-en-1-yl)benzonitrile (31 mg, 0.2 mmol), NaBH(OAc)₃ (11 mg, 0.3 mmol) and AcOH (one drop) were added to 10 mL DCE in 50 mL RBF, the mixture stirred at room temperature under N₂ atmosphere overnight. The reaction was quenched by adding 1 N NaOH (10 mL), and the product was extracted with CH₂Cl₂. The combined organic layers were washed with brine and dried over Na₂SO₄. The solvent was evaporated, and the crude product was purified by column chromatography (5% MeOH in EtOAc) to give compound 3 as a yellow powder (49 mg, 55% yield).

Compound 3 (89 mg, 0.2 mmol, methyl (S)-2-((tert-butoxycarbonyl)amino)-4-oxobutanoate 4 (55 mg, 0.24 mmol), NaBH(OAc)₃ (63 mg, 0.3 mmol) and AcOH (one drop) were added to 10 mL DCE in 50 mL RBF, the mixture stirred at room temperature under N₂ atmosphere overnight. The reaction was quenched by adding 1 N NaOH (10 mL), and the product was extracted with CH₂Cl₂. The combined organic layers were washed with brine and dried over Na₂SO₄. The solvent was evaporated, and the crude product was purified by column chromatography (5% MeOH in EtOAc) to give compound 5 as a white powder (94 mg, 71% yield).

Compound 5 (50 mg) was added to 10 mL TFA/DCM (1:1) and the mixture was stirred for 15 minutes at room temperature. The solvent was removed to offer compound 6, which was used directly in the next step without further purification.

3-(2-acetoxy-4,6-dimethylphenyl)-3-methylbutanoic acid 7 (20 mg, 0.075 mmol), BOP·PF₆ (33 mg, 0.075 mmol), intermediate compound 6 (51 mg, 0.075 mmol) and 0.1 mL Et₃N were added to 10 mL DCM in a 25 mL RBF. The mixture was stirred at room temperature for 2 hours before the reaction was quenched by adding 10 mL water. The product was extracted with CH₂Cl₂ and the combined organic layers were washed with brine and dried over Na₂SO₄. The solvent was evaporated and the crude product was purified by column chromatography (1% MeOH in EtOAc) to give compound 8 as a white powder (33 mg, 55% yield over 2 steps).

Compound 8 (30 mg, 0.037 mmol) was dissolved in 1 mL of CH₂Cl₂ and 9 mL TFA and 1 mL H₂O were added. The mixture was stirred for 2 hours at room temperature. The mixture was concentrated, and the crude product was purified by preparative HPLC affording final compound GYZ-573 as a white powder (17 mg, 59% yield). ¹H NMR (400 MHz, CD₃OD) δ 8.47 (s, 1H), 8.33 (s, 1H), 7.69 (d, J=8.4 Hz, 2H), 7.51 (d, J=8.5 Hz, 2H), 6.94 (s, 1H), 6.84 (br, 2H), 6.49 (dt, J=15.8, 7.2 Hz, 1H), 6.17 (d, J=3.4 Hz, 1H), 4.70 (dd, J=4.8, 3.4 Hz, 1H), 4.60-4.53 (m, 2H), 4.25 (dd, J=7.4, 5.7 Hz, 1H), 4.14 (d, J=7.3 Hz, 2H), 3.92-3.85 (m, 1H), 3.84 (s, 3H), 3.74-3.66 (m, 1H), 3.64-3.47 (m, 2H), 2.58-2.47 (m, 1H), 2.46-2.34 (m, 6H), 2.29 (s, 3H), 2.26 (s, 3H), 1.41 (s, 6H). ¹³C NMR (101 MHz, CD₃OD) δ 169.0, 168.3, 161.84, 161.5, 151.6, 148.2, 146.1, 139.75, 145.5, 143.0, 139.8, 138.4, 133.1, 132.3, 127.2, 119.8, 118.191.2, 78.7, 73.5, 72.2, 55.5, 54.9, 52.8, 50.2, 49.8, 49.5, 29.7, 24.9, 21.6, 20.3. HRMS (ESI): calculated for C₄₀H₄₈N₈O₈ [M+H]⁺ 769.3673, found 769.3682.

TABLE 2
HRMS and LRMS results for bisubstrate inhibitors with alkene linked
aromatics

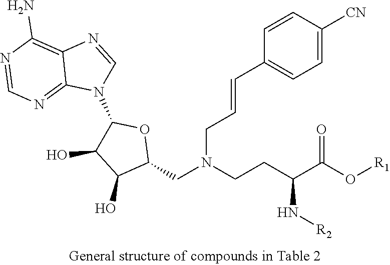

| | prodrug | | HRMS | |
|---|---|---|---|---|
| Code | Ester (R₁) | Amine (R₂) | calculated | found |
| GYZ-494 | methyl | H | [M + H]⁺ 523.2417 | 523.2415 |
| GYZ-552 | ethyl | H | [M + H]⁺ 537.2574 | 537 (LRMS) |
| GYZ-579 | propyl | H | [M + H]⁺ 551.2730 | 551 (LRMS) |
| GYZ-553 | isopropyl | H | [M + H]⁺ 551.2730 | 551 (LRMS) |
| GYZ-529 | benzyl | H | [M + H]⁺ 559.2730 | 559 (LRMS) |
| MH128 | H | TML | [M + H]⁺ 755.3511 | 755 (LRMS) |
| GYZ-573 | methyl | TML | [M + H]⁺ 769.3673 | 769.3682 |
| NB60 | Ethyl | TML | [M + H]⁺ 783.3830 | 783 (LRMS) |
| GYZ-580 | Propyl | TML | [M + H]⁺ 797.3986 | 797.3988 |
| NB61 | isopropyl | TML | [M + H]⁺ 797.3986 | 797 (LRMS) |
| GYZ-575 | benzyl | TML | [M + H]⁺ 845.3986 | 845.3991 |
| GYZ-672 | isopropyl | TML | [M + H]⁺ 772.4034 | 772.4041 |
| (p-CN=H) | | | | |

### Example 3

Compounds with Other Linkers to an Aromatic Group

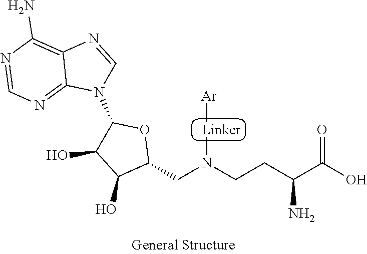

This section includes bisubstrate inhibitors with different aromatic groups linked via a 1-carbon alkyl, 3-carbon alkyl or 3 carbon alkyne spacer to the adenosine-amino acid scaffold.

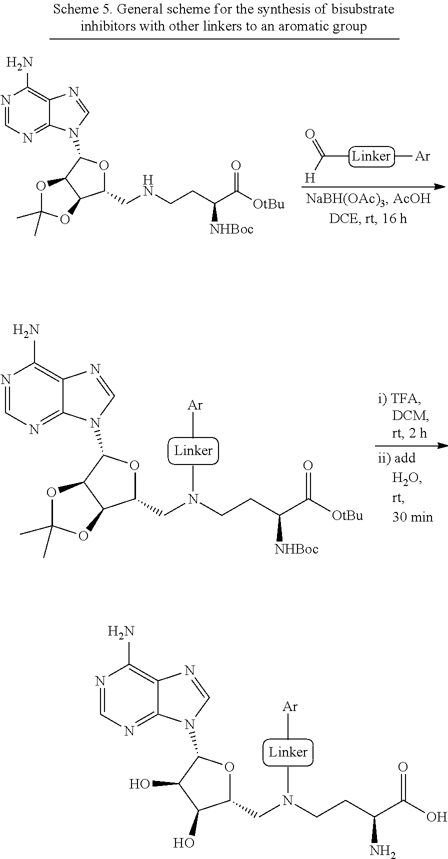

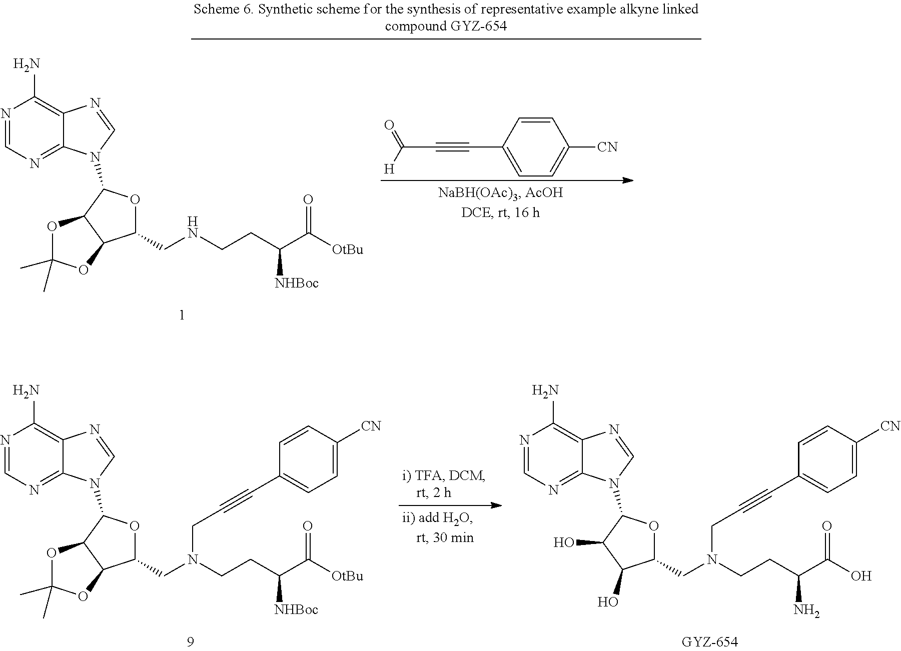 [SMILES: CC(C)(C)OC(=O)N[C@@H](CCNC[C@H]1O[C@@H](n2cnc3c(N)ncnc32)[C@@H]2OC(C)(C)O[C@@H]21)C(=O)OC(C)(C)C ; N#Cc1ccc(C#CC=O)cc1 ; CC(C)(C)OC(=O)N[C@@H](CCN(CC#Cc1ccc(C#N)cc1)C[C@H]1O[C@@H](n2cnc3c(N)ncnc32)[C@@H]2OC(C)(C)O[C@@H]21)C(=O)OC(C)(C)C ; N#Cc1ccc(C#CCN(CC[C@H](N)C(=O)O)C[C@H]2O[C@@H](n3cnc4c(N)ncnc43)[C@H](O)[C@@H]2O)cc1]

### Experimental Procedure for the Preparation of Representative Example Compound GYZ-654

Compound 1 (112 mg, 0.2 mmol), 4-(3-oxoprop-1-yn-1-yl)benzonitrile (37 mg, 0.24 mmol), NaBH(OAc)3 (11 mg, 0.3 mmol) and AcOH (one drop) were added to 10 mL DCE in a 50 mL RBF. The mixture was stirred at room temperature under N2 atmosphere overnight. The reaction was quenched by adding 1 N NaOH (10 mL), and the product was extracted with CH2Cl2. The combined organic layers were washed with brine and dried over Na2SO4. The solvent was evaporated and the crude product was purified by column chromatography (5% MeOH in EtOAc) to give intermediate 9 as a white powder (104 mg, 74% yield).

To a solution of compound 9 (50 mg, 0.074 mmol) in 1 mL of CH2Cl2 was added 9 mL TFA and 1 mL H2O and the mixture was stirred for 2 hours at room temperature. The mixture was concentrated, and the crude product was purified by preparative HPLC affording compound GYZ-654 as a white powder (36 mg, 81% yield). 1H NMR (500 MHz, CD3OD) δ 8.48 (s, 1H), 8.37 (s, 1H), 7.73 (d, J=8.6 Hz, 2H), 7.59 (d, J=8.6 Hz, 2H), 6.13 (d, J=4.1 Hz, 1H), 4.74-4.71 (m, 1H), 4.48-4.41 (m, 2H), 4.19 (s, 2H), 4.12 (t, J=6.4 Hz, 1H), 3.53-3.42 (m, 2H), 3.35 (d, J=6.5 Hz, 2H), 2.39-2.32 (m, 1H), 2.20-2.13 (m, 1H). 13C NMR (126 MHz, CD3OD) δ 170.5, 161.0, 151.50, 148.4, 132.1, 126.6, 117.8, 115.3, 112.2, 90.2, 86.2, 84.2, 73.7, 72.2, 55.9, 52.2, 51.1, 42.7, 25.8. HRMS (ESI): calculated for C24H26N8O5 [M+H]+ 507.2104, found 507.2113.

TABLE 3
IC₅₀ data and HRMS results for bisubstrate inhibitors with 1-carbon alkyl linkers to a
substituted phenyl group

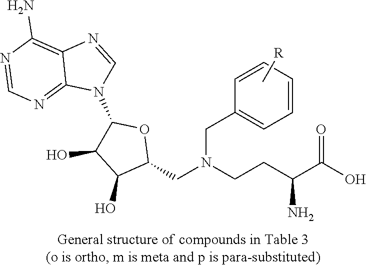

| | | | | Standard | | | |
|---|---|---|---|---|---|---|---|
| | | IC₅₀ values | | error | | HRMS | |
| Code | R | (μM) | (nM) | (μM) | (nM) | calculated | found |
| GYZ-421 | o-F | >25 | >25000 | n/a | n/a | [M + H]⁺ | 476.2063 |
| | | | | | | 476.2058 | |
| GYZ-422 | m-F | 1.24 | 1235.00 | 0.07 | 73.38 | [M + H]⁺ | 476.2067 |
| | | | | | | 476.2058 | |
| GYZ-423 | p-F | >25 | >25000 | n/a | n/a | [M + H]⁺ | 476.2070 |
| | | | | | | 476.2058 | |
| GYZ-415 | o-CF₃ | 9.70 | 9703.00 | 0.34 | 343.69 | [M + H]⁺ | 526.2034 |
| | | | | | | 526.2026 | |
| GYZ-416 | m-CF₃ | 6.58 | 6581.00 | 0.49 | 487.18 | [M + H]⁺ | 526.2027 |
| | | | | | | 526.2026 | |
| GYZ-414 | p-CF₃ | 6.79 | 6792.00 | 0.38 | 377.26 | [M + H]⁺ | 526.2026 |
| | | | | | | 526.2026 | |
| GYZ-425 | o-NO₂ | >25 | >25000 | n/a | n/a | [M + H]⁺ | 503.2011 |
| | | | | | | 503.2003 | |
| GYZ-419 | m-NO₂ | 0.32 | 320.20 | 0.03 | 30.90 | [M + H]⁺ | 503.2008 |
| | | | | | | 503.2003 | |
| GYZ-420 | p-NO₂ | 6.61 | 6609.00 | 0.35 | 346.79 | [M + H]⁺ | 503.2005 |
| | | | | | | 503.2003 | |
| GYZ-448 | p-CN | 2.78 | 2776.00 | 0.11 | 106.00 | [M + H]⁺ | 483.2110 |
| | | | | | | 483.2104 | |
| GYZ-760 | p-SO₂F | 5.62 | 5621.97 | 0.81 | 809.60 | [M + H]⁺ | 540.1682 |
| | | | | | | 540.1677 | |
| GYZ-417 | m-NO₂- | 0.41 | 407.30 | 0.02 | 21.02 | [M + H]⁺ | 521.1916 |
| | p-F | | | | | 521.1908 | |

### Alkyne-Linked Compounds:

TABLE 4
IC₅₀ data and HRMS results for bisubstrate inhibitors with 3-carbon alkynyl
linkers to a substituted phenyl group

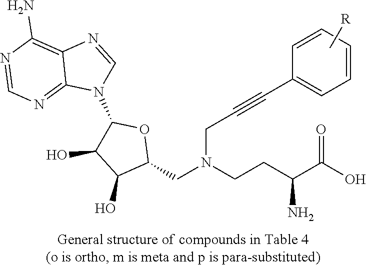

| | | IC₅₀ values | | Standard error | | HRMS | |
|---|---|---|---|---|---|---|---|
| Code | R | (μM) | (nM) | (μM) | (nM) | calculated | found |
| GYZ- | m-amide* | 0.010 | 10.23 | 0.00 | 0.897 | [M + H]⁺ | 525.2218 |
| 655 | | | | | | 525.2210 | |
| GYZ- | p-amide | >25 | >25000 | n/a | n/a | [M + H]⁺ | 525.2223 |
| 656 | | | | | | 525.2210 | |
| GYZ- | m-CN | 1.427 | 1427 | 0.10 | 101.642 | [M + H]⁺ | 507.2108 |
| 653 | | | | | | 507.2104 | |
| GYZ- | p-CN | 0.069 | 69.29 | 0.00 | 4.422 | [M + H]⁺ | 507.2113 |
| 654 | | | | | | 507.2104 | |

*GYZ-655 is a reference compound previously described in D. Chen et al., ″Novel Propargyl-Linked Bisubstrate Analogues as Tight-Binding Inhibitors for Nicotinamide N-Methyltransferase″, J. Med. Chem. 2019, 62, 23, 10783-10797.

Fully Saturated Alkane-Linked Compounds for Comparison with Published Structures:

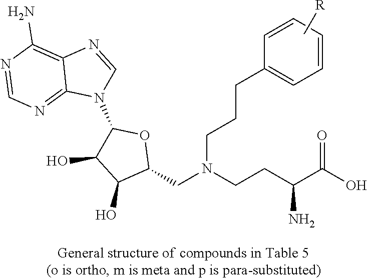

TABLE 5
IC₅₀ data and HRMS results for bisubstrate inhibitors with
3-carbon alkyl linkers to a substituted phenyl group

| | | IC₅₀ values | | Standard error | | HRMS | |
|---|---|---|---|---|---|---|---|
| Code | R | (μM) | (nM) | (μM) | (nM) | calculated | found |
| GYZ- | p-NO₂ | 0.29 | 294.44 | 0.03 | 31.55 | [M + H]⁺ | 531.2320 |
| 447 | | | | | | 531.2316 | |
| GYZ- | p-CN | 0.05 | 53.83 | 0.00 | 4.20 | [M + H]⁺ | 511.2428 |
| 451 | | | | | | 511.2417 | |
| GYZ- | m- | 0.08 | 83.03 | 0.00 | 3.32 | [M + H]⁺ | 529.2522 |
| 644 | amide* | | | | | 529.2513 | |

*GYZ-644 is a reference compound previously described in D. Chen et al., “Novel Propargyl-Linked Bisubstrate Analogues as Tight-Binding Inhibitors for Nicotinamide N-Methyltransferase”, J. Med. Chem. 2019, 62, 23, 10783-10797

### Example 4

Compounds where the Amino Acid Moiety has been Replaced

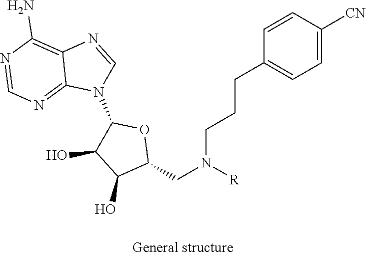

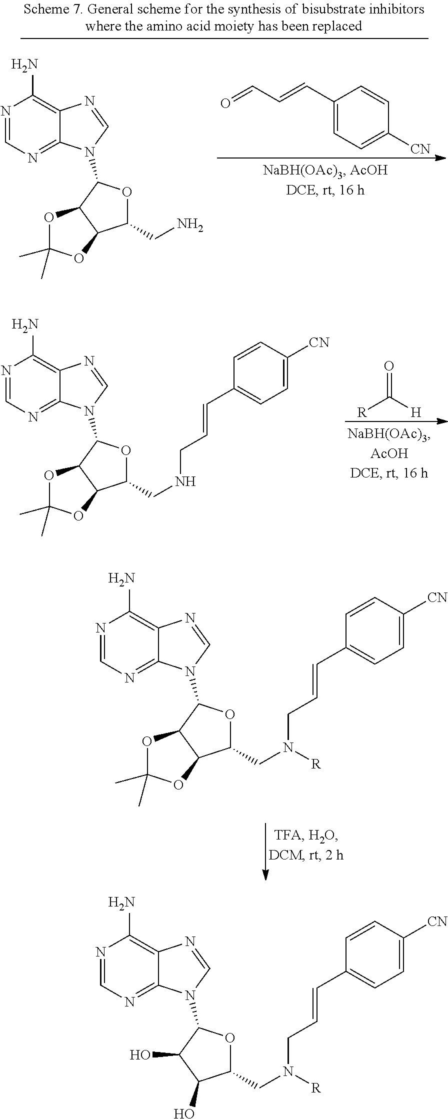

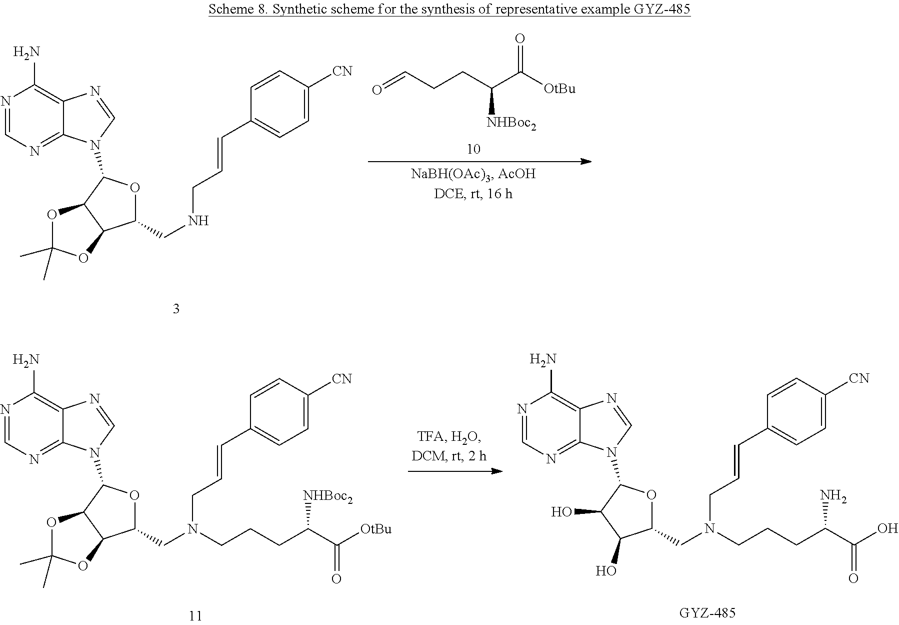

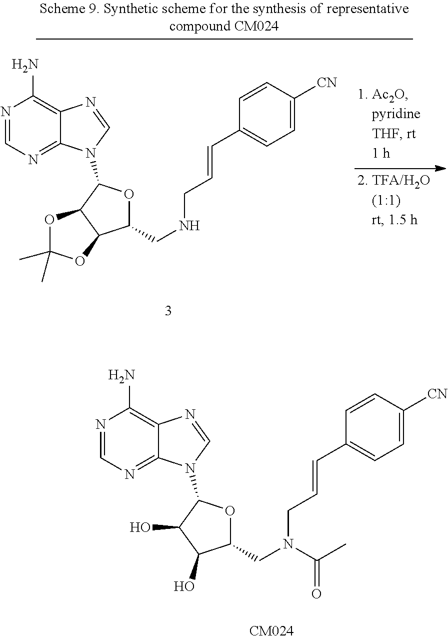 [SMILES: CC1(C)O[C@@H]2[C@H](O1)[C@@H](CNC/C=C/c1ccc(C#N)cc1)O[C@H]2n1cnc2c(N)ncnc21 ; CC(=O)N(C/C=C/c1ccc(C#N)cc1)C[C@H]1O[C@@H](n2cnc3c(N)ncnc32)[C@H](O)[C@@H]1O]

### Experimental Procedure for the Preparation of Representative Compound GYZ-485

4-((E)-3-((((3aR,4R,6R,6aR)-6-(6-amino-9H-purin-9-yl)-2,2-dimethyltetrahydrofuro[3,4-d][1,3]dioxol-4-yl)methyl)amino)prop-1-en-1-yl)benzonitrile 3 (89 mg, 0.2 mmol, tert-butyl (S)-2-(bis(tert-butoxycarbonyl)amino)-5-oxopentanoate 10 (82 mg, 0.24 mmol), NaBH(OAc)₃ (11 mg, 0.3 mmol) and AcOH (one drop) were added to DCE (10 mL) in a 50 mL RBF. The mixture was stirred at room temperature under N₂ atmosphere overnight before the reaction was quenched by adding 1 N NaOH (10 mL). The product was extracted with CH₂Cl₂ and the combined organic layers were washed with brine and dried over Na₂SO₄. The solvent was evaporated, and the crude product was purified by column chromatography (5% MeOH in EtOAc) to give compound 11 as a white powder (113 mg, 69% yield).

To a solution of compound 11 (50 mg, 0.061 mmol) in 1 mL of CH₂Cl₂ was added 9 mL TFA and 1 mL H₂O. The mixture was stirred for 2 h at room temperature and concentrated. The crude product was purified by preparative HPLC affording compound GYZ-485 as a white powder (24 mg, 63% yield). ¹H NMR (400 MHz, CD₃OD) δ 8.47 (s, 1H), 8.33 (s, 1H), 7.68 (d, J=8.3 Hz, 2H), 7.48 (d, J=8.2 Hz, 2H), 6.80 (br, 1H), 6.51-6.43 (m, 1H), 6.18 (d, J=3.2 Hz, 1H), 4.71-4.65 (m, 1H), 4.62-4.51 (m, 2H), 4.19-4.00 (m, 3H), 3.89-3.84 (m, 1H), 3.69 (br, 1H), 3.47-3.37 (m, 2H), 2.13-1.91 (m, 4H). ¹³C NMR (101 MHz, CD₃OD) δ 170.0, 151.3, 148.1, 145.0, 143.1, 139.7, 138.5, 132.3, 127.2, 120.0, 119.7, 118.1, 118.0, 115.1, 111.9, 91.3, 73.5, 72.2, 55.3, 52.8, 27.1, 20.0. HRMS (ESI): calculated for C₂₅H₃₀N₈O₅[M+H]⁺ 523.2417, found 523.2423.

### Experimental Procedure for the Preparation of Representative Example Compound CM024

Intermediate 3 (120 mg, 0.26 mmol) was dissolved in THF (4 mL) and pyridine (30 mg, 0.4 mmol, 1.5 eq) was added, followed by acetic anhydride (30 mg, 0.30 mmol, 1.1 eq). The mixture was stirred for 1 hour after which full conversion was observed by LC-MS.

The mixture was concentrated, redissolved in a mixture of TFA and water (2:1, 9 mL) and the mixture stirred at rt. After 1.5 hours, LCMS indicates full conversion and the mixture is concentrated and purified by preparative HPLC yielding 56 mg (48%) pure product CM024 as a mixture of rotamers. ¹H NMR (500 MHz, DMSO) δ 8.67 and 8.61 (s, 1H), 8.38 (s, 1H), 7.81-7.67 (m, 2H), 7.60-7.47 (m, 2H), 6.56-6.29 (m, 2H), 5.96 (d, J=4.4 Hz) and 5.92 (d, J=5.9 Hz) (1H), 4.76-4.58 (m, 2H), 4.26-4.02 (m, 4H), 3.94-3.83 (m, 1H), 3.73-3.61 (m, 1H), 3.50-3.35 (m, 1H), 2.04 and 2.00 (s, 3H). ¹³C NMR (126 MHz, DMSO) δ 170.42, 169.92, 158.87, 158.59, 152.46, 152.21, 148.73, 148.58, 141.25, 141.02, 132.53, 130.20, 130.07, 129.29, 128.99, 127.03, 126.92, 119.09, 119.04, 118.95, 118.94, 109.72, 109.56, 88.42, 87.71, 82.95, 82.69, 73.06, 72.93, 71.59, 71.18, 51.00, 50.12, 47.99, 46.99, 21.53, 21.50. Calculated mass for C22H23N7O4 [M+H]+=450.1884, LRMS found 450.2.

TABLE 6
IC₅₀ data and HRMS results for bisubstrate inhibitors with an amino acid replacement

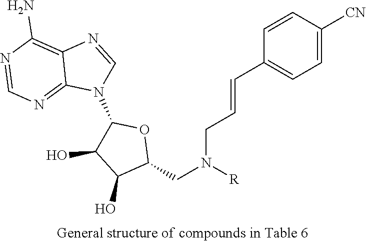

| | | IC₅₀ values | | Standard error | | HRMS | |
|---|---|---|---|---|---|---|---|
| Code | R | (μM) | (nM) | (μM) | (nM) | calculated | found |
| NPB-3 | 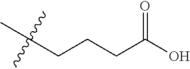 [SMILES: *CCCC(=O)O] | >25 | >25000 | n/a | n/a | [M + H]⁺ 494.2152 | 494 (LRMS) |
| GYZ- 538 | 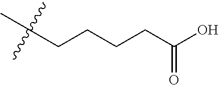 [SMILES: *CCCCC(=O)O] | >25 | >25000 | n/a | n/a | [M + H]⁺ 508.2308 | 508 (LRMS) |
| NPB-2 | 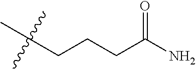 [SMILES: *CCCC(N)=O] | >25 | >25000 | n/a | n/a | [M + H]⁺ 493.2312 | 493 (LRMS) |
| GYZ- 539 | 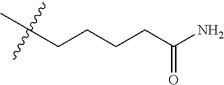 [SMILES: *CCCCC(N)=O] | >25 | >25000 | n/a | n/a | [M + H]⁺ 507.2468 | 507 (LRMS) |
| GYZ- | methyl | >25 | >25000 | n/a | n/a | [M + H]⁺ | 422 |
| 523 | | | | | | 422.1941 | (LRMS) |
| GYZ- | isopropyl | >25 | >25000 | n/a | n/a | [M + H]⁺ | 450 |
| 449 | | | | | | 450.2254 | (LRMS) |
| GYZ- | H | 4.78 | 4780.79 | 0.66 | 655.93 | [M + H]⁺ | 408.1796 |
| 450 | | | | | | 408.1784 | |
| GYZ- 498 | 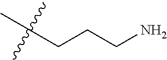 [SMILES: *CCCN] | 0.96 | 957.30 | 0.09 | 85.46 | [M + H]⁺ 465.2363 | 465.2369 |
| GYZ- 540 | 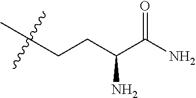 [SMILES: *CC[C@H](N)C(N)=O] | 1.901 | 1901 | 0.23 | 231.44 | [M + H]⁺ 508.2421 | 508 (LRMS) |
| GYZ- 466 | 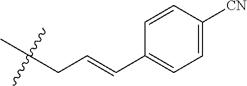 [SMILES: *C/C=C/c1ccc(C#N)cc1] | >25 | >25000 | n/a | n/a | [M + H]⁺ 49.2363 | 549.2372 |
| GYZ- 485 | 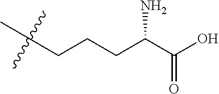 [SMILES: *CCC[C@H](N)C(=O)O] | 0.36 | 355.55 | 0.07 | 73.13 | [M + H]⁺ 523.2417 | 523.2420 |
| GYZ- 718 | 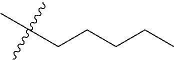 [SMILES: *CCCCC] | >25 | >25000 | n/a | n/a | [M + H]⁺ 478.2567 | 478.2565 |
| GYZ- 761 | 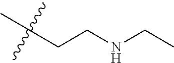 [SMILES: *CCNCC] | >25 | >25000 | n/a | n/a | [M + H]⁺ 479.2519 | 479.2522 |
| GYZ- 756 | 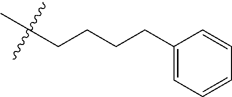 [SMILES: *CCCCc1ccccc1] | >25 | >25000 | n/a | n/a | [M + H]⁺ 540.2723 | 540.2728 |
| GYZ- 605 | 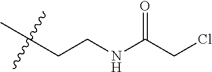 [SMILES: *CCNC(=O)CCl] | 0.4 | 396.0 | 0.01 | 8.63 | [M + H]⁺ 527.1922 | 527.1931 |
| GYZ- 608 | 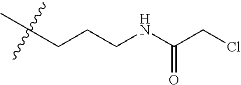 [SMILES: *CCCNC(=O)CCl] | 11.86 | 11860 | 0.53 | 527.03 | [M + H]⁺ 541.2079 | 541.2083 |
| GYZ- 604 | 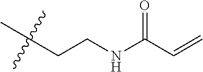 [SMILES: *CCNC(=O)C=C] | 20.98 | 20980 | 1.06 | 1060.28 | [M + H]⁺ 505.2312 | 505 (LRMS) |
| GYZ- 627 | 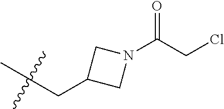 [SMILES: *CC1CN(C(=O)CCl)C1] | 19.77 | 19770 | 0.96 | 956.38 | [M + H]⁺ 553.2079 | 553 (LRMS) |
| GYZ- 665 | 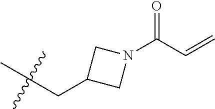 [SMILES: *CC1CN(C(=O)C=C)C1] | 4.157 | 4157 | 0.32 | 320.16 | [M + H]⁺ 531.2468 | 531 (LRMS) |
| GYZ- 663 | 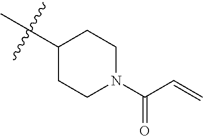 [SMILES: *C1CCN(C(=O)C=C)CC1] | 8.46 | 8460 | 0.40 | 395.58 | [M + H]⁺ 559.2781 | 559 (LRMS) |
| GYZ- 748 | 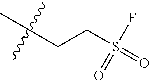 [SMILES: *CCS(=O)(=O)F] | >25 | >25000 | n/a | n/a | [M + H]⁺ 518.1622 | 518.1627 |
| GYZ- 702 | 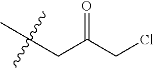 [SMILES: *CC(=O)CCl] | 0.98 | 980 | 0.08 | 81.11 | [M + H]⁺ 498.1657 | 498 (LRMS) |
| GYZ- 704 | 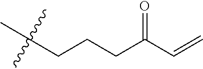 [SMILES: *CCCC(=O)C=C] | 0.538 | 538 | 0.03 | 34.86 | [M + H]⁺ 504.2359 | 504 (LRMS) |
| GYZ- 693 | 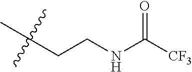 [SMILES: *CCNC(=O)C(F)(F)F] | 7.766 | 7766.022 | 0.583 | 582.865 | [M + H]⁺ 547.2029 | 547.2035 |
| GYZ- 694 | 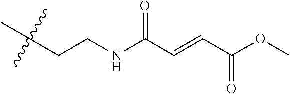 [SMILES: *CCNC(=O)/C=C/C(=O)OC] | >25 | >25000 | n/a | n/a | [M + H]⁺ 563.2367 | 563.2371 |
| GYZ- 695 | 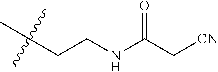 [SMILES: *CCNC(=O)CC#N] | 0.931 | 930.799 | 0.063 | 63.462 | [M + H]⁺ 518.2264 | 518.2274 |
| GYZ- 701 | 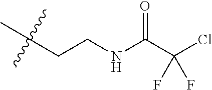 [SMILES: *CCNC(=O)C(F)(F)Cl] | >25 | >25000 | n/a | n/a | [M + H]⁺ 563.1734 | 563.1745 |
| GYZ- 706 | 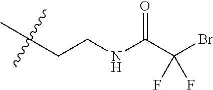 [SMILES: *CCNC(=O)C(F)(F)Br] | >25 | >25000 | n/a | n/a | [M + H]⁺ 607.1228 | 607.1239 |
| GYZ- 743 | 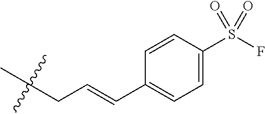 [SMILES: *C/C=C/c1ccc(S(=O)(=O)F)cc1] | 7.776 | 7776.497 | 0.82 | 820.33 | [M + H]⁺ 606.1935 | 606.1942 |
| CM024 | acetyl | >25 | >25000 | n/a | n/a | [M + H]⁺ | LRMS |
| | | | | | | 450.1884 | 450.2 |

### Example 5

General structure of the adenosyl moiety indicating potential sites of heteroatom variation:

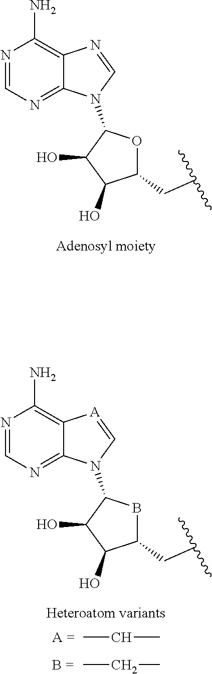

Compounds comprising the adenosyl moiety heteroatom variants illustrated above may be synthesised by the skilled person by straightforward adaptation of the methods disclosed herein, e.g. using commercially available reagents.

### Example 6

### MTT Results for Exemplary Prodrugs

TABLE 7
MTT results of compound 319 and exemplary corresponding
prodrugs. Values are presented as % of the DMSO
control after 24, 48 or 72 hours of incubation.

| | % of DMSO control at 100 μM | | | | | | | | |
|---|---|---|---|---|---|---|---|---|---|
| | HSC-2 | | | T24 | | | A549 | | |
| prodrug | 24 h | 48 h | 72 h | 24 h | 48 h | 72 h | 24 h | 48 h | 72 h |
| GYZ- | 100 | 53 | 49 | 100 | 79 | 86 | 100 | 74 | 66 |
| 319 | | | | | | | | | |
| GYZ- | 100 | 100 | 87 | 100 | 76 | 100 | 100 | 100 | 88 |
| 494 | | | | | | | | | |
| GYZ- | 100 | 100 | 73 | 100 | 100 | 100 | 100 | 100 | 100 |
| 552 | | | | | | | | | |
| GYZ- | 100 | 57 | 63 | 82 | 71 | 100 | 100 | 100 | 100 |
| 579 | | | | | | | | | |
| GYZ- | 100 | 75 | 56 | 100 | 71 | 100 | 100 | 100 | 100 |
| 553 | | | | | | | | | |
| GYZ- | 100 | 71 | 56 | 100 | 72 | 77 | 100 | 100 | 100 |
| 529 | | | | | | | | | |
| MH128 | 100 | 100 | 65 | 100 | 83 | 84 | 100 | 100 | 100 |
| GYZ- | 83 | 69 | 39 | 100 | 79 | 62 | 100 | 100 | 100 |
| 573 | | | | | | | | | |
| NB60 | 79 | 44 | 26 | 100 | 50 | 29 | 100 | 100 | 76 |
| GYZ- | 59 | 41 | 16 | 72 | 36 | 15 | 83 | 45 | 25 |
| 580 | | | | | | | | | |

### Example 7

### Comparative Inhibition Data

Table 8 shows activity data performed in the presence of normal and elevated concentrations of cofactor SAM.

General structure in Table 8, where S is “substitution” and “L” is linker:

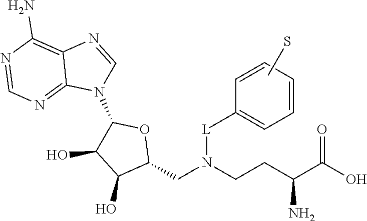

TABLE 8
IC₅₀ values for para-cyano compounds GYZ-319 and GYZ654 and
meta-amide compounds GYZ567 and GYZ-655 in the presence of SAM at
its K_{M} value of 8.5 μM or 10 times its K_{M} value (85 μM)

| | | | IC₅₀ in nM with s.e.m. | |
|---|---|---|---|---|
| Compound | substitution | linker | 8.5 μM SAM | 85 μM SAM |
| GYZ-319 | para-CN | propenyl | 3.69 ± 0.17 | 16.00 ± 1.48 |
| GYZ-654 | para-CN | propargyl | 69.29 ± 4.42 | 258.25 ± 26.21 |
| GYZ-567 | meta-amide | propenyl | 12.76 ± 0.78 | 39.53 ± 4.52 |
| GYZ-655 | meta-amide | propargyl | 10.23 ± 0.90 | 21.66 ± 1.61 |

### Example 8

### Synthesis of Deazodeoxy Compounds

The first part of the synthesis provided a chloro or amino substituted intermediate. Commercially available 2-(4,6-dichloropyrimidin-5-yl)acetaldehyde (12) was reacted with triethylorthoformate in the presence of a catalytic amount of toluenesulfonic acid in ethanol at 40° C. to form 4,6-dichloro-5-(2,2-diethoxyethyl)pyrimidine (13). intermediate 13 was subsequently coupled to (1S,2R,3S,5S)-3-amino-5-(hydroxymethyl)-cyclopentane-1,2-diol hydrochloride salt using triethylamine in a mixture of isopropanol and water (7:1) stirred at 90° C. for 16 hours after which the mixture was cooled to 50° C. and cyclized by addition of 5 M HCl and stirring for 2 hours. Intermediate 14 was protected using 2,2-dimethoxypropane and toluenesulfonic acid in acetone in 2 hours at 60° C. Intermediate 15 was converted to the chloro-substituted intermediate 17 through a Mitsunobu reaction with triphenylphosphine, di-tert-butylazodicarboxylate and diphenylphosphoryl azide and subsequent reduction of the resulting azide 16 to the corresponding amine using trimethylphosphine. Alternatively, intermediate 15 was converted to the amino-substituted intermediate 20 through amination using ammonium hydroxide in 1,4-dioxane at 95° C. for 16 hours in a sealed tube, followed by a Mitsunobu reaction with triphenylphosphine, di-tert-butylazodicarboxylate and phthalimide and subsequent removal of the phthalimide using methylamine in ethanol. Intermediate 17 could also be directly converted to intermediate 20 through amination using ammonium hydroxide in 1,4-dioxane at 95° C. for 16 hours in a sealed tube.

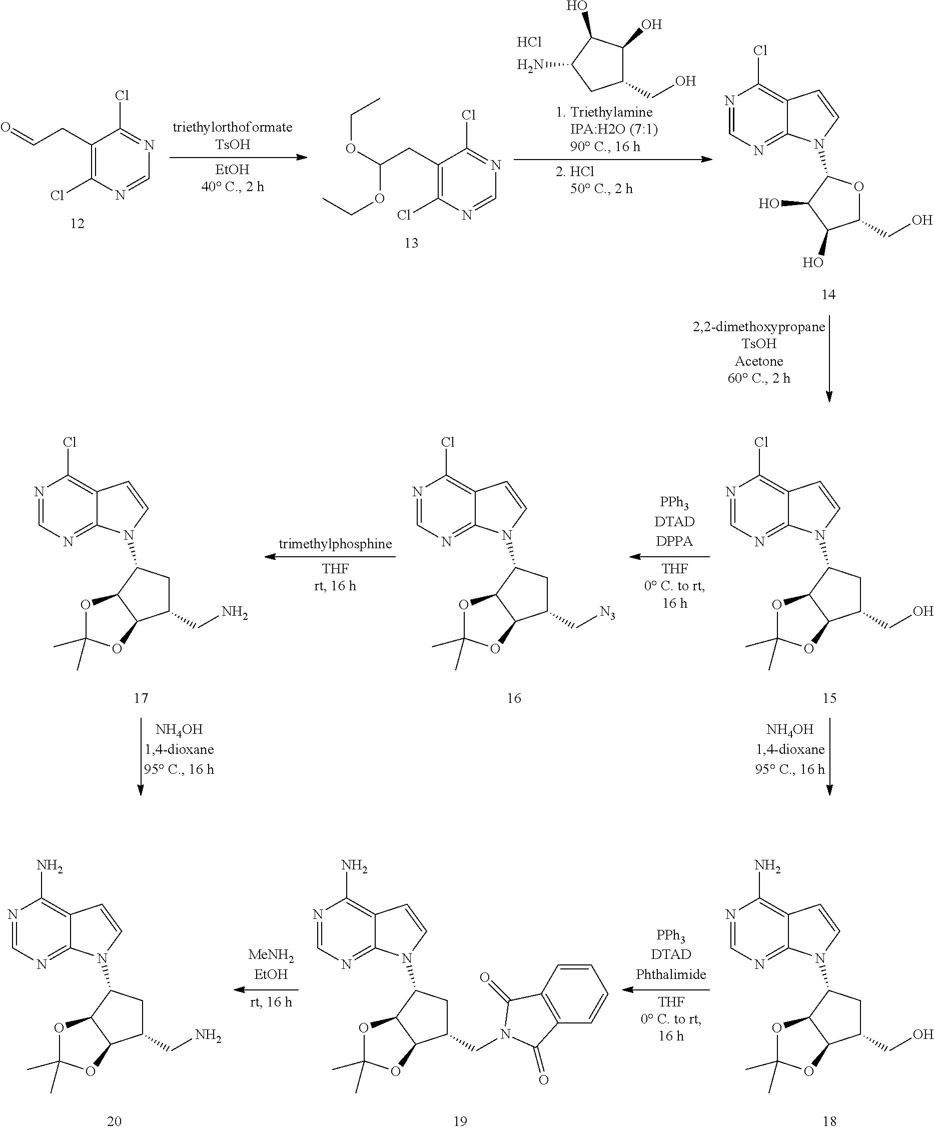

The final compound was then made according to the following scheme:

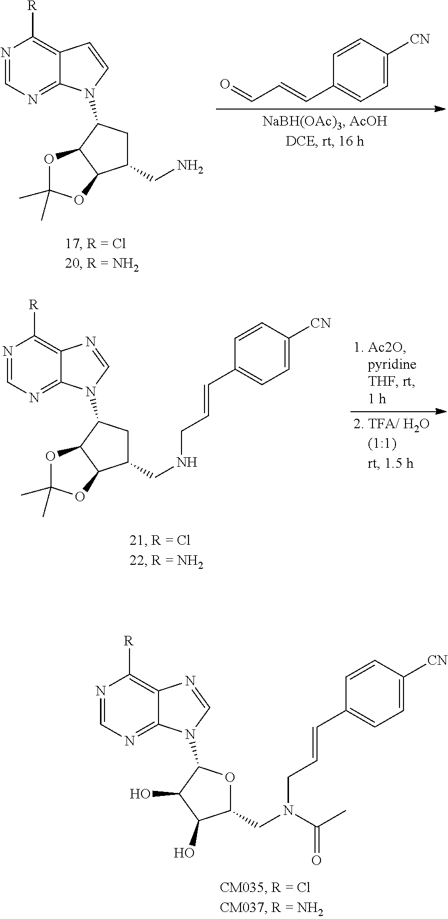

Experimental procedure for the Preparation of Representative Example Compound CM035

Intermediate 17 (74 mg, 0.23 mmol) and 4-cyanocinnamaldehyde (35 mg, 0.22 mmol) were dissolved in DCE (10 mL), followed by the addition of a drop of acetic acid. After 2 hours, NaBH(OAc)3 (70 mg, 0.33 mmol) is added and the mixture is then stirred at room temperature under nitrogen atmosphere for 16 hours. The mixture is quenched with 1M NaOH (25 mL) and extracted with DCM (3×20 mL), washed with brine, dried with Na2SO4 and concentrated. The crude product is purified by column chromatography starting with EtOAc, followed by 10% MeOH in EtOAc to give intermediate 21 (23 mg, 23%) as a yellowish oil. Intermediate 21 (23 mg, 0.05 mmol) was subsequently dissolved in dry THF (1 mL) and pyridine (11 mg, 0.15 mmol) was added followed by a solution of acetic anhydride (6 mg, 0.06 mmol) in dry THF (2 mL). Full conversion was observed after 1 hour stirring at room temperature. The mixture was concentrated and redissolved in 4 mL TFA/H2O (2:1), stirred for 90 minutes at room temperature, concentrated and purified by preparative HPLC to yield 12 mg (52%) pure product CM035 as a mixture of rotamers. 1H NMR (400 MHz, MeOD) δ 8.54 and 8.53 (s, 1H), 7.78-7.39 (m, 5H), 6.73-6.36 (m, 3H), 5.15-4.96 (m, 1H), 4.58-4.45 (m, 1H), 4.35-4.21 (m, 2H), 4.07-3.95 (m, 1H), 3.86-3.71 (m, 1H), 3.61-3.45 (m, 1H), 2.57-2.31 (m, 2H), 2.26 and 2.17 (s, 3H), 2.07-1.72 (m, 1H). 13C NMR (101 MHz, MeOD) δ 174.28, 152.52, 150.91, 142.55, 133.59, 133.53, 131.89, 131.50, 131.00, 130.35, 130.23, 130.14, 128.31, 128.16, 119.74, 119.24, 111.99, 100.40, 100.27, 76.57, 76.07, 74.39, 74.22, 63.10, 62.01, 52.62, 51.73, 44.12, 43.17, 31.98, 31.82, 21.67. Calculated mass for C₂₄H₂₄ClN₅O₃[M+H]+=466.1640, LRMS found 466.2.

### Experimental Procedure for the Preparation of Representative Example Compound CM037

Intermediate 20 (90 mg, 0.30 mmol) and 4-cyanocinnamaldehyde (48 mg, 0.30 mmol) were dissolved in DCE (10 mL), followed by the addition of a drop of acetic acid. After 2 hours, NaBH(OAc)3 (70 mg, 0.33 mmol) is added and the mixture is then stirred at room temperature under nitrogen atmosphere for 16 hours. The mixture is quenched with 1M NaOH (35 mL) and extracted with DCM (3×30 mL), washed with brine, dried with Na2SO4 and concentrated. The crude product is purified by column chromatography with a gradient of 5% to 20% MeOH in EtOAc to give intermediate 22 as a yellowish oil. Intermediate 22 (15 mg, 0.04 mmol) was subsequently dissolved in dry THF (1 mL) and pyridine (6 mg, 0.08 mmol) was added followed by a solution of acetic anhydride (6 mg, 0.06 mmol) in dry THF (2 mL). Full conversion was observed after 1 hour stirring at room temperature. The mixture was concentrated and redissolved in 4 mL TFA/H2O (2:1), stirred for 90 minutes at room temperature, concentrated and purified by preparative HPLC to yield 9 mg (58%) pure product CM037 as a mixture of rotamers. 1H NMR (400 MHz, MeOD) δ 8.22 and 8.21 (s, 1H), 7.73-7.47 (m, 5H), 6.95-6.83 (m, 1H), 6.70-6.36 (m, 2H), 5.18-4.95 (m, 1H), 4.52-4.38 (m, 1H), 4.34-4.19 (m, 2H), 4.04-3.91 (m, 1H), 3.84-3.65 (m, 1H), 3.60-3.42 (m, 1H), 2.56-2.29 (m, 2H), 2.24 and 2.18 (s, 3H), 1.89-1.68 (m, 1H). 13C NMR (101 MHz, MeOD) δ 174.32, 142.55, 142.29, 133.60, 133.54, 131.89, 131.54, 130.24, 130.11, 128.33, 128.17, 127.73, 127.30, 119.72, 112.02, 102.83, 102.72, 76.94, 76.44, 74.30, 74.02, 62.72, 61.82, 52.56, 51.64, 44.22, 43.16, 32.44, 32.12, 21.72, 21.66. Calculated mass for C₂₄H₂₆N₆O₃[M+H]+=447.2139, LRMS found 447.2.

TABLE 9
IC₅₀ data and HRMS results for compounds CM035 and CM037

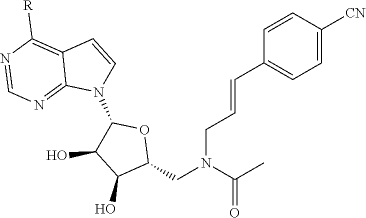

| | | IC₅₀ values | | Standard error | | HRMS | |
|---|---|---|---|---|---|---|---|
| Code | R | (μM) | (nM) | (μM) | (nM) | calculated | found |
| CM035 | Cl | 3.58 | 3578.51 | 0.89 | 888.78 | [M + H]⁺ | 466.2 |
| | | | | | | 466.1640 | (LRMS) |
| CM037 | NH₂ | 0.26 | 263.92 | 0.05 | 45.62 | [M + H]⁺ | 447.2 |
| | | | | | | 447.2139 | (LRMS) |

1. A compound of formula I, 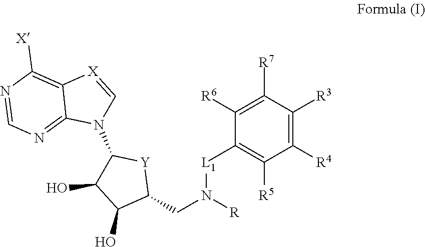
wherein:
X is selected from N and CH;
X′ is selected from NH₂ and halo;
Y is selected from O and CH₂;
L₁ is selected from the group consisting of: C₂-C₅ alkyl, C₃-C₅ alkenyl and C₃-C₅ alkynyl;
R is selected from the group consisting of: 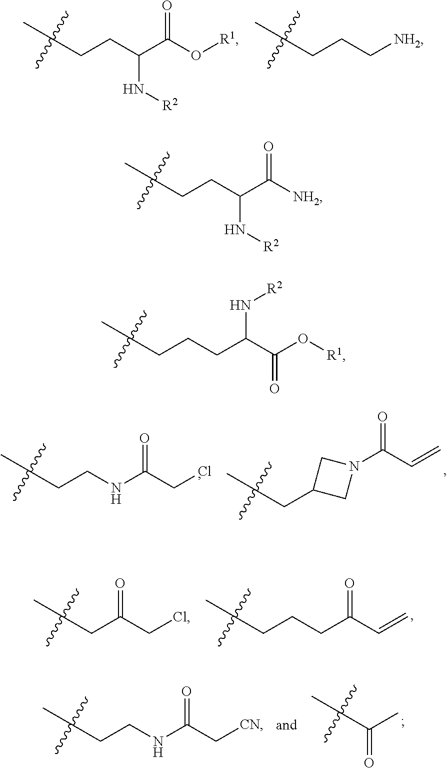
R¹ is selected from the group consisting of: H and a masking group;
R² is selected from the group consisting of: H and a masking group;
R³ is selected from the group consisting of: H and an electron withdrawing group;
when R³ is an electron withdrawing group, R⁴ is selected from H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g}; and when R³ is H, R⁴ is selected from C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g};
R⁵, R⁶ and R⁷ are each independently selected from H, C₁-C₆ alkyl, C₂-C₆ alkenyl, C₂-C₆ alkynyl, C₁-C₆ haloalkyl, halo, NO₂, CN, OR^{a}, CH₂OR^{a}, SR^{a}, CH₂SR^{a}, C(O)R^{b}, C(O)OR^{b}, C(O)NR^{c}R^{d}, S(O)₂R^{e} and NR^{f}R^{g};
R^{a}, R^{b}, R^{c}, R^{d}, R^{e}, R^{f} and R^{g} are each independently at each occurrence selected from the group consisting of: H, halo, C₁-C₃ alkyl, C₂-C₅ alkenyl and C₁-C₃ haloalkyl;
or a pharmaceutically acceptable salt, stereoisomer, solvate or prodrug thereof.

2. The compound of claim 1, wherein L₁ is an unsubstituted C₃-C₅ alkenyl, optionally wherein L₁ is —CH₂CHCH—.

3. (canceled)

4. The compound of claim 1, wherein R³ is an electron withdrawing group.

5. The compound of claim 1, wherein R is selected from 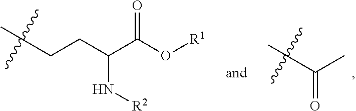
optionally wherein R is selected from

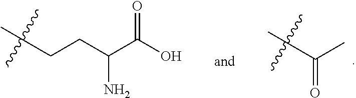

6. The compound of claim 1, wherein the compound is a compound of formula II, 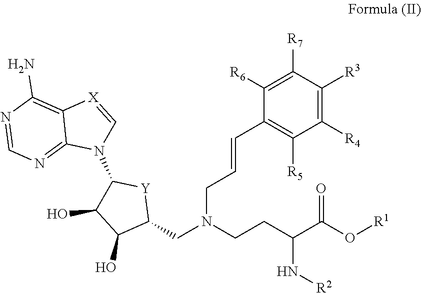
or a pharmaceutically acceptable salt, stereoisomer, solvate or prodrug thereof.

7. The compound of claim 1, wherein X is N; and/or wherein Y is O.

8. (canceled)

9. The compound of claim 1, wherein R¹ is selected from the group consisting of: H and a masking group, wherein the masking group is substituted or unsubstituted C₁-C₆ alkyl, lipids, substituted or unsubstituted benzyl, substituted or unsubstituted aryl, and

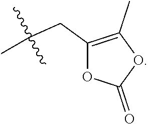 [SMILES: *Cc1oc(=O)oc1C]

10. (canceled)

11. The compound of claim 1, wherein R¹ is selected from the group consisting of methyl, ethyl, propyl, isopropyl, benzyl and H.

12. (canceled)

13. (canceled)

14. The compound of claim 1, wherein R² is H.

15. The compound of claim 1, wherein R² is a masking group, optionally wherein the masking group is selected from the group consisting of: 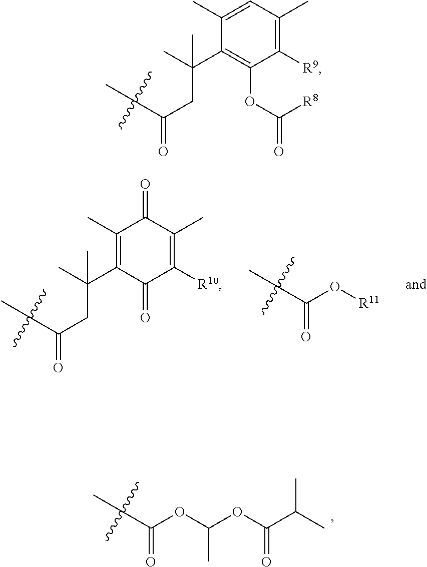
wherein
R⁸ is C₁-C₆ alkyl or aryl; R⁹ is H or methyl; R¹⁰ is H or methyl; and R¹¹ is C₁-C₆ alkyl or aryl.

16. (canceled)

17. The compound of claim 1, wherein R³ is selected from the group consisting of: halo, CN, NO₂, CF₃ and SO₂F.

18. The compound of claim 1, wherein R³ is CN.

19. The compound of claim 1, wherein R⁴, R⁵, R⁶ and R⁷ are each independently selected from H, haloalkyl, halo, NO₂, CN and C(O)NR^{c}R^{d}.

20. (canceled)

21. (canceled)

22. The compound of claim 1, wherein each of R⁴, R⁵, R⁶ and R⁷ are halo (optionally F), or H.

23. (canceled)

24. (canceled)

25. The compound of claim 1, wherein the compound is a compound selected from: 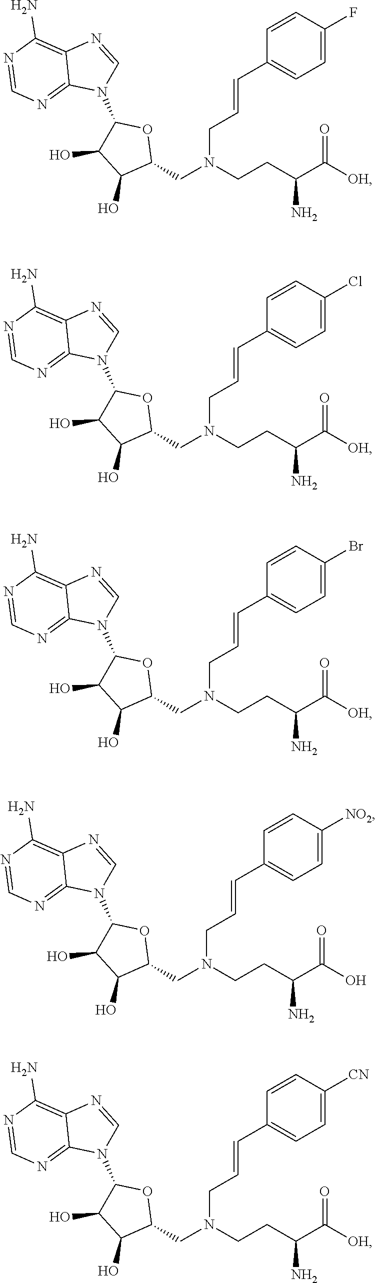 [SMILES: Nc1ncnc2c1ncn2[C@@H]1O[C@H](CN(C/C=C/c2ccc(F)cc2)CC[C@H](N)C(=O)O)[C@@H](O)[C@H]1O ; Nc1ncnc2c1ncn2[C@@H]1O[C@H](CN(C/C=C/c2ccc(Cl)cc2)CC[C@H](N)C(=O)O)[C@@H](O)[C@H]1O ; Nc1ncnc2c1ncn2[C@@H]1O[C@H](CN(C/C=C/c2ccc(Br)cc2)CC[C@H](N)C(=O)O)[C@@H](O)[C@H]1O ; Nc1ncnc2c1ncn2[C@@H]1O[C@H](CN(C/C=C/c2ccc([N+](=O)[O-])cc2)CC[C@H](N)C(=O)O)[C@@H](O)[C@H]1O ; N#Cc1ccc(/C=C/CN(CC[C@H](N)C(=O)O)C[C@H]2O[C@@H](n3cnc4c(N)ncnc43)[C@H](O)[C@@H]2O)cc1] 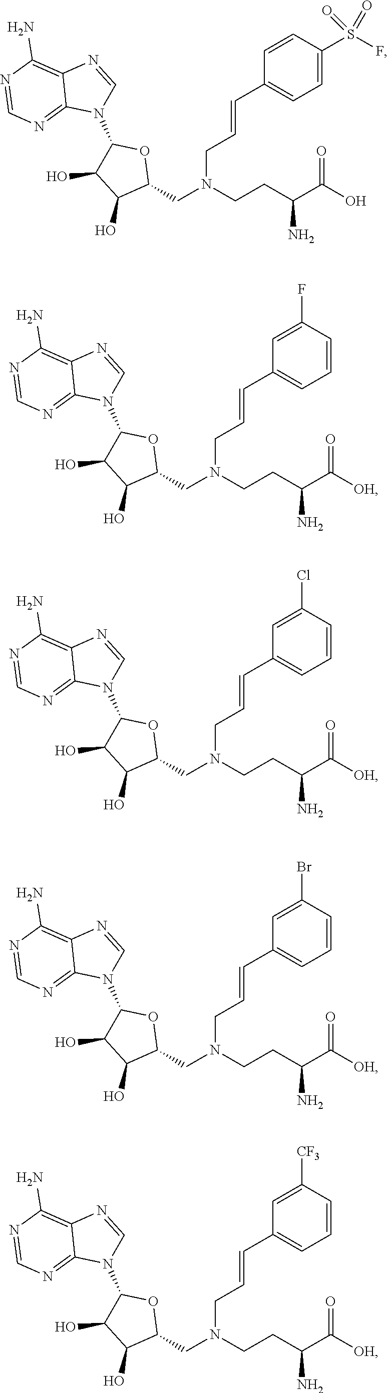 [SMILES: Nc1ncnc2c1ncn2[C@@H]1O[C@H](CN(C/C=C/c2ccc(S(=O)(=O)F)cc2)CC[C@H](N)C(=O)O)[C@@H](O)[C@H]1O ; Nc1ncnc2c1ncn2[C@@H]1O[C@H](CN(C/C=C/c2cccc(F)c2)CC[C@H](N)C(=O)O)[C@@H](O)[C@H]1O ; Nc1ncnc2c1ncn2[C@@H]1O[C@H](CN(C/C=C/c2cccc(Cl)c2)CC[C@H](N)C(=O)O)[C@@H](O)[C@H]1O ; Nc1ncnc2c1ncn2[C@@H]1O[C@H](CN(C/C=C/c2cccc(Br)c2)CC[C@H](N)C(=O)O)[C@@H](O)[C@H]1O ; Nc1ncnc2c1ncn2[C@@H]1O[C@H](CN(C/C=C/c2cccc(C(F)(F)F)c2)CC[C@H](N)C(=O)O)[C@@H](O)[C@H]1O] 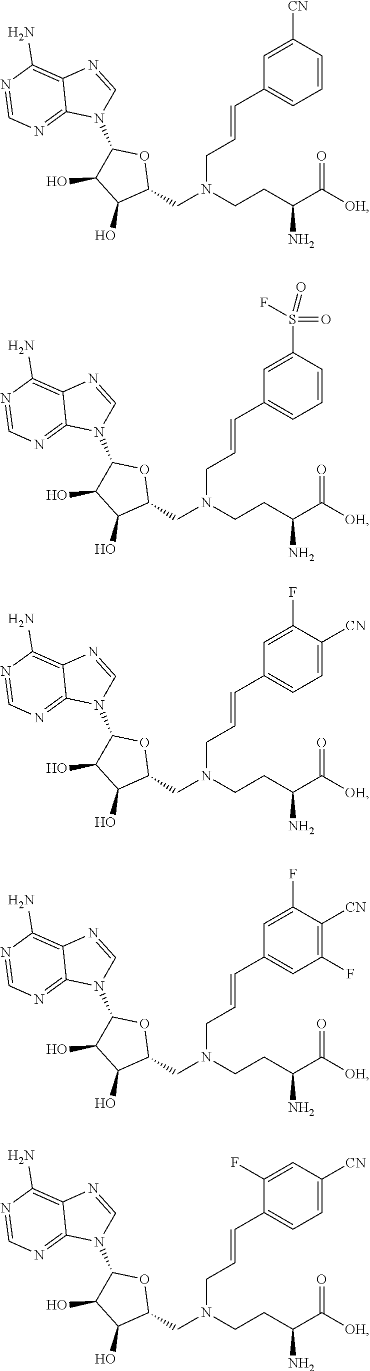 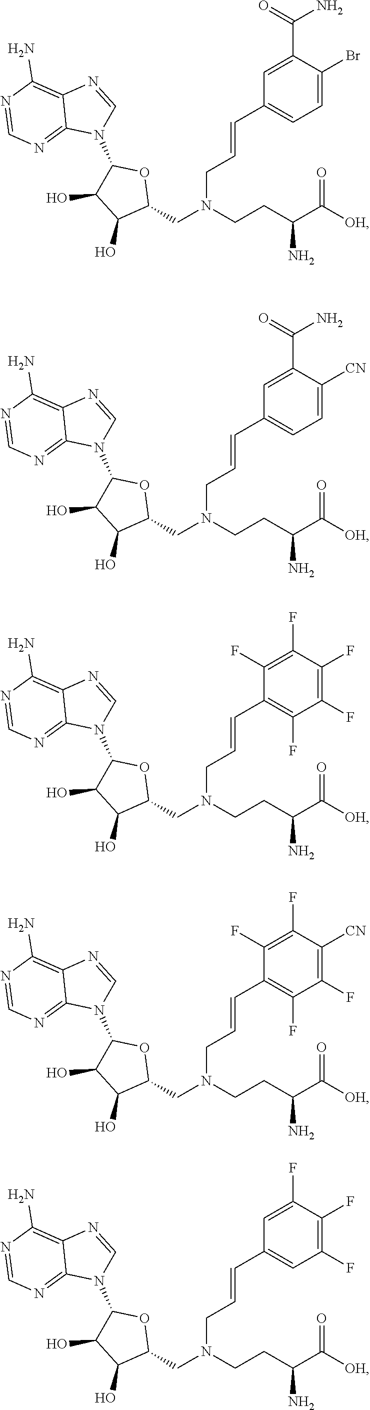 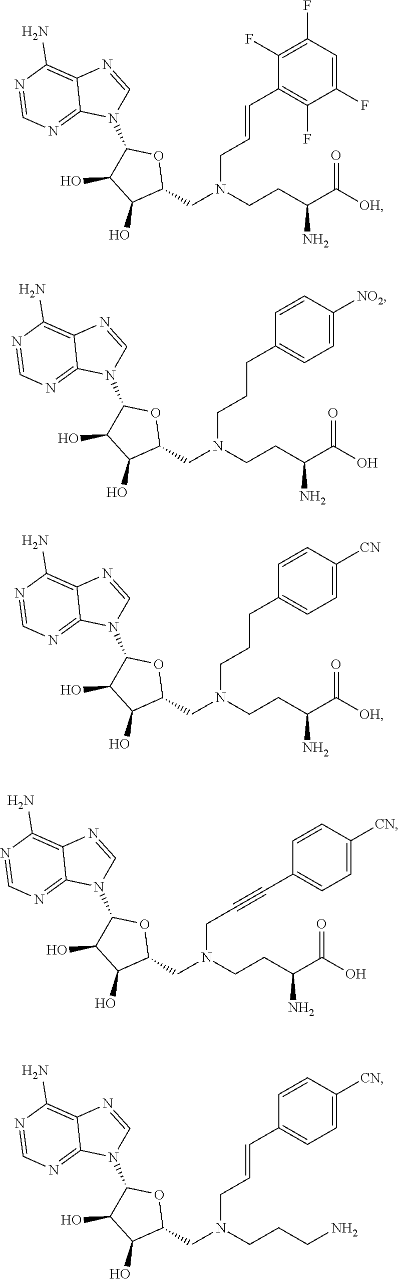 [SMILES: Nc1ncnc2c1ncn2[C@@H]1O[C@H](CN(C/C=C/c2c(F)c(F)cc(F)c2F)CC[C@H](N)C(=O)O)[C@@H](O)[C@H]1O ; Nc1ncnc2c1ncn2[C@@H]1O[C@H](CN(CCCc2ccc([N+](=O)[O-])cc2)CC[C@H](N)C(=O)O)[C@@H](O)[C@H]1O ; N#Cc1ccc(CCCN(CC[C@H](N)C(=O)O)C[C@H]2O[C@@H](n3cnc4c(N)ncnc43)[C@H](O)[C@@H]2O)cc1 ; N#Cc1ccc(C#CCN(CC[C@H](N)C(=O)O)C[C@H]2O[C@@H](n3cnc4c(N)ncnc43)[C@H](O)[C@@H]2O)cc1 ; N#Cc1ccc(/C=C/CN(CCCN)C[C@H]2O[C@@H](n3cnc4c(N)ncnc43)[C@H](O)[C@@H]2O)cc1] 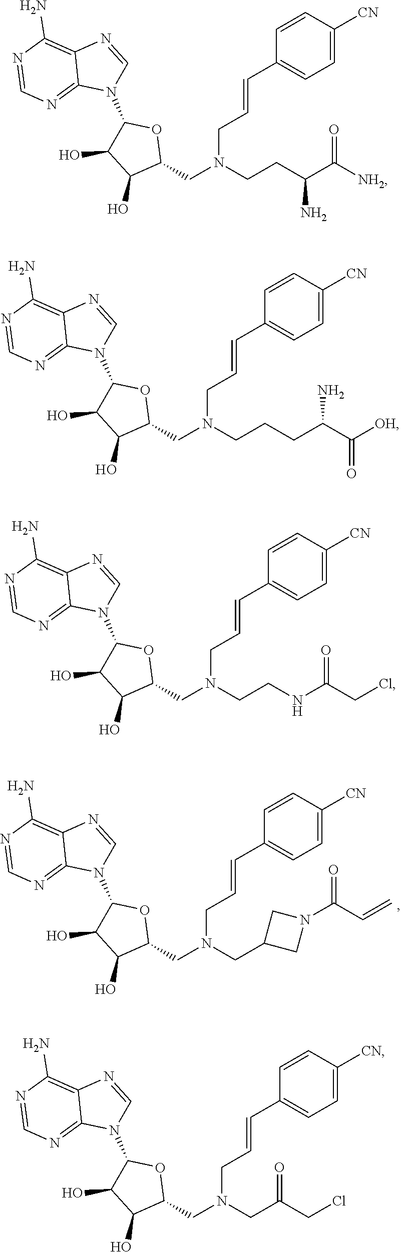 [SMILES: N#Cc1ccc(/C=C/CN(CC[C@H](N)C(N)=O)C[C@H]2O[C@@H](n3cnc4c(N)ncnc43)[C@H](O)[C@@H]2O)cc1 ; N#Cc1ccc(/C=C/CN(CCC[C@H](N)C(=O)O)C[C@H]2O[C@@H](n3cnc4c(N)ncnc43)[C@H](O)[C@@H]2O)cc1 ; N#Cc1ccc(/C=C/CN(CCNC(=O)CCl)C[C@H]2O[C@@H](n3cnc4c(N)ncnc43)[C@H](O)[C@@H]2O)cc1 ; C=CC(=O)N1CC(CN(C/C=C/c2ccc(C#N)cc2)C[C@H]2O[C@@H](n3cnc4c(N)ncnc43)[C@H](O)[C@@H]2O)C1 ; N#Cc1ccc(/C=C/CN(CC(=O)CCl)C[C@H]2O[C@@H](n3cnc4c(N)ncnc43)[C@H](O)[C@@H]2O)cc1] 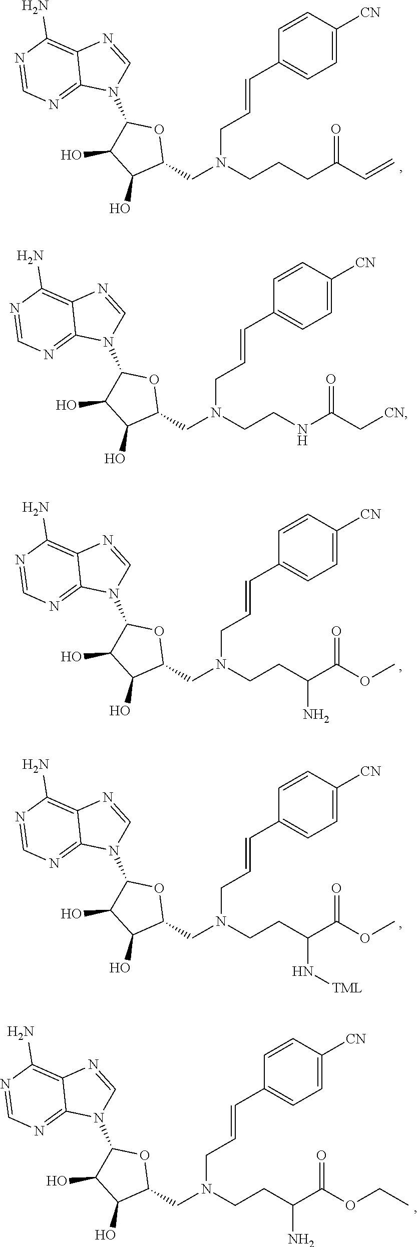 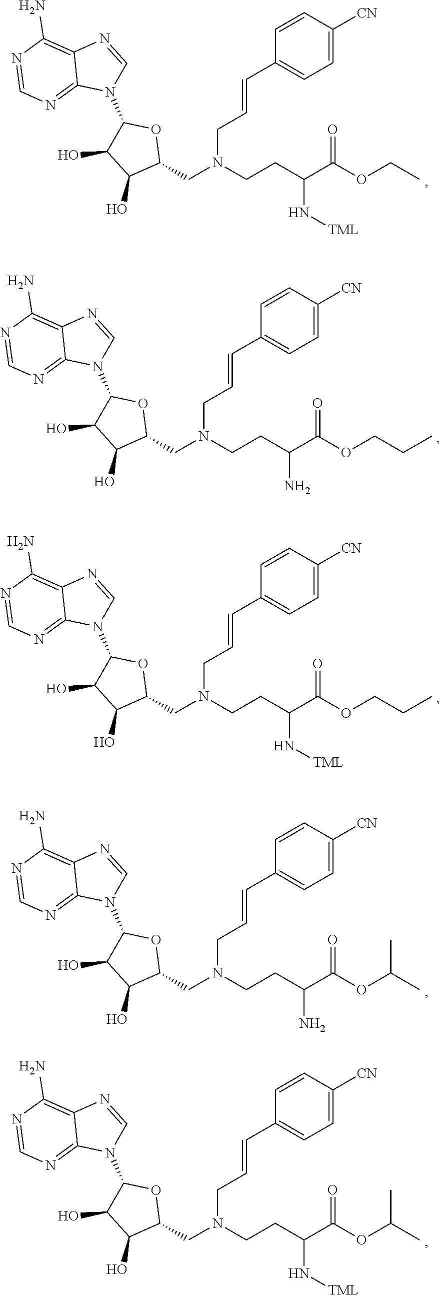 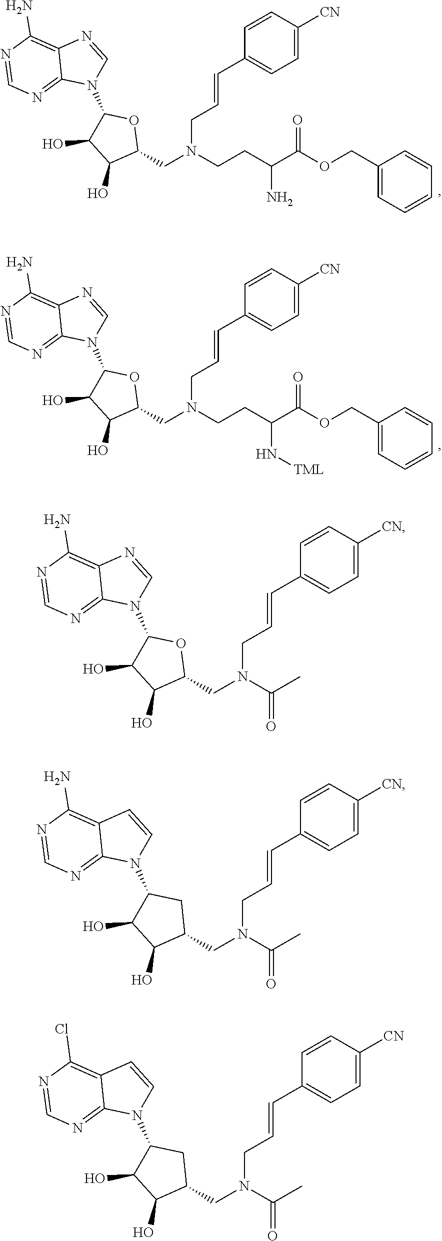
where TML is selected from 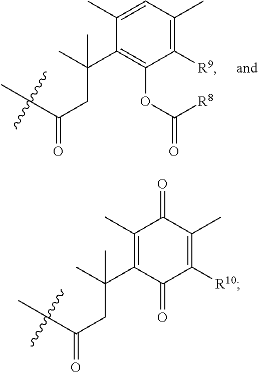
wherein R⁸ is C₁-C₆ alkyl or aryl, R⁹ is H or methyl; R¹⁰ is H or methyl; and R¹¹ is C₁-C₆ alkyl or aryl; or a pharmaceutically acceptable salt, stereoisomer, or solvate thereof.

26. A pharmaceutical formulation comprising a compound of claim 1 and optionally a pharmaceutically acceptable carrier.

27. (canceled)

28. (canceled)

29. (canceled)

30. (canceled)

31. (canceled)

32. (canceled)

33. (canceled)

34. (canceled)

35. A method of treatment of a condition which is modulated by the inhibition of NNMT, wherein the method comprises administering a therapeutic amount of a compound of claim 1, to a patient in need thereof.

36. The method of claim 35, wherein the condition is selected from the group consisting of: cancer (such as lung cancer, bladder cancer, renal cancer, oral cancer, skin cancer, breast cancer, colorectal cancer, gastric cancer, hepatocellular cancer, ovarian cancer, pancreatic cancer, prostate cancer and glioblastoma), metabolic disorders, metabolic syndrome, diabetes, neurodegenerative diseases, Alzheimer's disease, Parkinson's disease, Huntington's diseases, schizophrenia, functional disorders of the endothelium, thrombosis, high blood pressure, atherosclerosis, inflammation and pulmonary hypertension.

37. A method for the inhibition of NNMT, comprising administering a compound of claim 1 in vitro or in vivo.
